(19) United States
(12) Patent Application Publication
(10) Pub. No.: US 2025/0281645 A1
(43) Pub. Date: Sep. 11, 2025

(54) Lentiviral Vector

(71) Applicants: Ospedale San Raffaele S.r.l., Milan (IT); Fondazione Telethon ETS, Rome (IT)

(72) Inventors: Alessio Cantore, Milan (IT); Cesare Canepari, Milan (IT)

(21) Appl. No.: 18/860,059

(22) PCT Filed: Apr. 28, 2023
(86) PCT No.: PCT/EP2023/061389

§ 371 Date: Oct. 25, 2024

(30) Foreign Application Priority Data
Apr. 29, 2022 (IT) 102022000008690
Oct. 11, 2022 (GB) 2214973.6

(51) Int. Cl. A61K 48/00; A61K 31/69; A61K 38/17; A61P 3/06; C07K 16/28; C12N 15/86

(52) U.S. Cl. CPC A61K 48/0058; A61K 31/69; A61K 38/177; A61P 3/06; C07K 16/2866; C12N 15/86; C12N 2740/15043

(57) ABSTRACT

A lentiviral vector comprising a nucleotide sequence encoding low density lipoprotein receptor (LDLR) operably linked to a promoter, optionally wherein the nucleotide sequence encoding LDLR and the promoter are in a reverse orientation in the lentiviral vector.

### FIELD OF THE INVENTION

The present invention relates to lentiviral vectors comprising a nucleotide sequence encoding low density lipoprotein receptor (LDLR), and cells and pharmaceutical compositions comprising the lentiviral vectors. The invention further relates to uses of the lentiviral vectors in treating or preventing familial hypercholesterolemia (FH) and familial hypercholesterolemia associated conditions, such as atherosclerosis.

### BACKGROUND TO THE INVENTION

Familial hypercholesterolemia (FH) is an autosomal dominant inherited disorder, which affects an estimated 1 in 350 people, in its heterozygous form, and 1 in 300,000 people in its homozygous form (43,000 patients worldwide). Characterised by extremely high LDL cholesterol in the circulation, this condition leads to progressive atherosclerosis. If the homozygous form is left untreated, myocardial infarction usually develops within the first decade of life, leading to death within the third decade. More than 95% of mutations that result in this disorder occur in the gene encoding the low density lipoprotein receptor (LDLR). Other less frequently occurring mutations may be found in the ApoB100 and PCSK9 genes, the former a structural component of the LDL, the latter an inhibitor of the LDLR.

In recent years, much effort has been made to counteract the issue of high cholesterol and a plethora of successful drugs have been developed for its treatment. Examples include inhibitors of cholesterol absorption and statins; and monoclonal antibodies and short interfering RNA targeting PCSK9, which are aimed at increasing the amounts of LDLR on cell membranes, thus increasing clearance of cholesterol from the circulation. However, the situation is different for homozygous patients, which do not harbor a functional copy of LDLR. Current treatments with cholesterol-lowering drugs are only partially effective. Despite strategies targeting molecules involved in cholesterol metabolism showing promising results, they are, per se, unable to fully normalise cholesterol and are associated with significant side effects in homozygous patients (Bajaj et al. (2020) Current Opinion in Lipidology).

LDL apheresis is therefore presently required to reach LDL cholesterol target levels, and liver transplantation is currently the only curative option for homozygous patients. However, the liver is not the only organ responsible for clearance of cholesterol, and providing a patient with a liver with functional LDLR activity is not sufficient to fully normalise LDL levels. Moreover, due to the limited availability of compatible organs and the complications related to liver transplantation, new therapeutic options are required for treating FH.

LDLR gene transfer has been proposed as a gene therapy option for the treatment of FH using adeno-associated viral (AAV) vectors. However, the main limitation of this platform is that AAV vectors remain in cells mainly as episomes that are progressively lost upon cell division during liver growth, thus limiting their application to paediatric patients. To overcome this issue, integrating gene therapy approaches have also been proposed in proof-of-concept studies, based on lentiviral vectors (LVs) (Kankkonen et al. (2004) Mol Ther 9: 548-556). However, problems may exist with the production of sufficient amounts of LVs encoding LDLR and there remains a significant need for effective, long-term treatment options for FH.

In addition, high quantities of highly purified LV may be required to treat large animals, such as human beings. Improvements in the efficiency of LV gene transfer may reduce doses required to achieve a therapeutic outcome, both for non cell autonomous diseases (e.g. haemophilia) and even more for cell autonomous diseases, such as familial hypercholesterolemia.

### SUMMARY OF THE INVENTION

The present inventors have successfully developed LVs encoding LDLR that may be produced in significant titer and function as a gene therapy for FH. In particular, the inventors have produced LVs encoding a LDLR transgene and evaluated their function in vitro in hepatocyte cell lines and in vivo in a mouse model of FH. The LVs may specifically target and restrict the transgene expression to hepatocytes by combining transcriptional and post-transcriptional microRNA-mediated regulation to allow efficient and safe liver gene transfer; and may be engineered to lack allogeneic major histocompatibility complexes (MHC) and to bear high surface content of the phagocytosis inhibitor CD47. This therapeutic strategy may provide for stable expression of the therapeutic protein after a single administration to young patients, whose liver is actively growing, and thus whose hepatocyte proliferation is high.

In one aspect, the invention provides a lentiviral vector comprising a nucleotide sequence encoding low density lipoprotein receptor (LDLR). The nucleotide sequence encoding LDLR may be operably linked to a promoter, such as a liver-specific promoter (e.g. a hepatocyte-specific promoter).

In one aspect, the invention provides a lentiviral vector comprising a nucleotide sequence encoding low density lipoprotein receptor (LDLR) operably linked to a liver-specific promoter (e.g. a hepatocyte-specific promoter).

In some embodiments, the nucleotide sequence encoding LDLR is in a reverse orientation in the lentiviral vector. In some embodiments, the nucleotide sequence encoding LDLR and the promoter are in a reverse orientation in the lentiviral vector. In some embodiments, an expression cassette comprising the nucleotide sequence encoding LDLR operably linked to a promoter is in a reverse orientation in the lentiviral vector.

In some embodiments, the nucleotide sequence encoding LDLR is in a sense orientation in the lentiviral vector. In some embodiments, the nucleotide sequence encoding LDLR and the promoter are in a sense orientation in the lentiviral vector. In some embodiments, an expression cassette comprising the nucleotide sequence encoding LDLR operably linked to a promoter is in a sense orientation in the lentiviral vector.

In one aspect, the invention provides a lentiviral vector comprising a nucleotide sequence encoding low density lipoprotein receptor (LDLR), wherein the nucleotide sequence encoding LDLR is in a reverse orientation in the lentiviral vector.

In one aspect, the invention provides a lentiviral vector comprising a nucleotide sequence encoding low density lipoprotein receptor (LDLR) operably linked to a promoter, wherein the nucleotide sequence encoding LDLR and the promoter are in a reverse orientation in the lentiviral vector.

In one aspect, the invention provides a lentiviral vector comprising an expression cassette comprising a nucleotide sequence encoding low density lipoprotein receptor (LDLR) operably linked to a promoter, wherein the expression cassette is in a reverse orientation in the lentiviral vector.

In some embodiments, the LDLR comprises or consists of an amino acid sequence that has at least 70%, at least 80%, at least 90%, at least 95%, at least 96%, at least 97%, at least 98% or at least 99% sequence identity to SEQ ID NO: 1 or 38, preferably SEQ ID NO: 1, or a fragment thereof.

In some embodiments, the LDLR comprises or consists of an amino acid sequence that has at least 70% sequence identity to SEQ ID NO: 1 or 38, preferably SEQ ID NO: 1, or a fragment thereof.

In some embodiments, the LDLR comprises or consists of the amino acid sequence of SEQ ID NO: 1 or 38, preferably SEQ ID NO: 1, or a fragment thereof.

In some embodiments, the nucleotide sequence encoding LDLR comprises or consists of a nucleotide sequence that has at least 70%, at least 80%, at least 90%, at least 95%, at least 96%, at least 97%, at least 98% or at least 99% sequence identity to SEQ ID NO: 2 or 39, preferably SEQ ID NO: 2, or a fragment thereof.

In some embodiments, the nucleotide sequence encoding LDLR comprises or consists of a nucleotide sequence that has at least 70% sequence identity to SEQ ID NO: 2 or 39, preferably SEQ ID NO: 2, or a fragment thereof.

In some embodiments, the nucleotide sequence encoding LDLR comprises or consists of the nucleotide sequence of SEQ ID NO: 2 or 39, preferably SEQ ID NO: 2, or a fragment thereof.

The nucleotide sequences disclosed herein are shown in the forward orientation (5′ to 3′). When a nucleotide sequence (e.g. the nucleotide sequence encoding LDLR, the promoter, the miRNA target sequence(s) and/or the polyadenylation sequence) is comprised in a vector in the reverse orientation then the nucleotide sequence comprised in the vector will be the reverse complement of the nucleotide sequence shown herein. In some embodiments, when in the reverse orientation the nucleotide sequence encoding the LDLR is the reverse complement of the sequence shown for the corresponding SEQ ID NO (e.g. SEQ ID NO: 2 or 39), or variant thereof, of the disclosure. In some embodiments, when in the reverse orientation the nucleotide sequence of the promoter is the reverse complement of the sequence shown for the corresponding SEQ ID NO (e.g. SEQ ID NO: 24 or 25), or variant thereof, of the disclosure. In some embodiments, when in the reverse orientation the nucleotide sequence of the miRNA target sequence is the reverse complement of the sequence shown for the corresponding SEQ ID NO (e.g. SEQ ID NO: 6 or 7), or variant thereof, of the disclosure.

In some embodiments, the promoter is a liver-specific promoter. In some embodiments, the promoter is a hepatocyte-specific promoter.

In some embodiments, the promoter is selected from the group consisting of a transthyretin (TTR) promoter, an alpha-1-antityrpsin (AAT) promoter, a thyroxine-binding globulin (TBG) promoter, a APoE/hAAT promoter, a HCR-hAAT promoter, a LP1 promoter and a HLP promoter.

In some embodiments, the promoter is a transthyretin (TTR) promoter. In some embodiments, the promoter is an enhanced transthyretin (ET) promoter.

In some embodiments, the promoter comprises or consists of a nucleotide sequence that has at least 70%, at least 80%, at least 90%, at least 95%, at least 96%, at least 97%, at least 98% or at least 99% sequence identity to SEQ ID NO: 24 or 25, preferably SEQ ID NO: 24.

In some embodiments, the promoter comprises or consists of a nucleotide sequence that has at least 70% sequence identity to SEQ ID NO: 24 or 25, preferably SEQ ID NO: 24.

In some embodiments, the promoter comprises or consists of the nucleotide sequence of SEQ ID NO: 24 or 25, preferably SEQ ID NO: 24.

In some embodiments, the lentiviral vector is in the form of a lentiviral vector particle.

In some embodiments, the lentiviral vector is comprised in a non-viral particle, for example a nanoparticle, lipid nanoparticle or liposome.

In some embodiments, the lentiviral vector is a VSV.G-pseudotyped lentiviral vector.

In some embodiments, the lentiviral vector is a self-inactivating (SIN) lentiviral vector.

In some embodiments, the lentiviral vector is an integrating lentiviral vector. In some embodiments, the lentiviral vector is a replication-defective lentiviral vector. In some embodiments, the lentiviral vector is an integrating lentiviral vector and a replication-defective lentiviral vector.

In some embodiments, the nucleotide sequence encoding LDLR is operably linked to one or more miRNA target sequence.

In some embodiments, the one or more miRNA target sequence suppresses LDLR expression in one or more cell type other than hepatocytes. In some embodiments, the one or more miRNA target sequence suppresses LDLR expression in hematopoietic-lineage cells and/or antigen-presenting cells.

In some embodiments, the one or more miRNA target sequence is selected from the group consisting of a miR-142 target sequence, a miR-181 target sequence, a miR-223 target sequence and a miR-155 target sequence.

In some embodiments, the nucleotide sequence encoding LDLR is operably linked to one or more miR-142 target sequence. In some embodiments, the nucleotide sequence encoding LDLR is operably linked to two or more miR-142 target sequences. In some embodiments, the nucleotide sequence encoding LDLR is operably linked to three or more miR-142 target sequences. In some embodiments, the nucleotide sequence encoding LDLR is operably linked to four or more miR-142 target sequences.

In some embodiments, the nucleotide sequence encoding LDLR is operably linked to four miR-142 target sequences.

In some embodiments, the one or more miRNA target sequence comprises or consists of a nucleotide sequence that has at least 90%, at least 95%, at least 96%, at least 97%, at least 98% or at least 99% sequence identity to SEQ ID NO: 6.

In some embodiments, the one or more miRNA target sequence comprises or consists of a nucleotide sequence that has at least 90% sequence identity to SEQ ID NO: 6.

In some embodiments, the one or more miRNA target sequence comprises or consists of the nucleotide sequence of SEQ ID NO: 6.

In some embodiments, the one or more miRNA target sequence comprises or consists of a nucleotide sequence that has at least 90%, at least 95%, at least 96%, at least 97%, at least 98% or at least 99% sequence identity to SEQ ID NO: 7.

In some embodiments, the one or more miRNA target sequence comprises or consists of a nucleotide sequence that has at least 90% sequence identity to SEQ ID NO: 7.

In some embodiments, the one or more miRNA target sequence comprises or consists of the nucleotide sequence of SEQ ID NO: 7.

In some embodiments, the lentiviral vector is a CD47^{high} lentiviral vector.

In some embodiments, the lentiviral vector is obtained from a CD47^{high} producer cell. In some embodiments, the producer cell is genetically engineered to increase expression of CD47 on the cell surface.

In some embodiments, the lentiviral vector has at least about 2-fold more CD47 on its surface than a lentiviral vector obtained from an unmodified producer cell.

In some embodiments, the lentiviral vector is a MHC-I^{free} lentiviral vector.

In some embodiments, the lentiviral vector is obtained from a MHC-I^{free} producer cell. In some embodiments, the producer cell is genetically engineered to disrupt expression of MHC-I on the cell surface.

In some embodiments, MHC-I is not detectable on the surface of the lentiviral vector.

In some embodiments, the lentiviral vector is a CD47^{high}/MHC-I^{free} lentiviral vector.

In some embodiments, the lentiviral vector is obtained from a CD47^{high}/MHC-I^{free} producer cell.

In one aspect, the invention provides an isolated cell comprising a lentiviral vector of the invention.

In some embodiments, the cell is a hepatocyte.

In one aspect, the invention provides use of the lentiviral vector of the invention for transducing a population of cells.

In some embodiments, the use is an in vitro or ex vivo use.

In one aspect, the invention provides a method of transducing a population of cells, the method comprising contacting the population of cells with the lentiviral vector of the invention.

In some embodiments, the method is an in vitro or ex vivo method.

In some embodiments, the population of cells comprises or consists of a population of hepatocytes.

In one aspect, the invention provides a pharmaceutical composition comprising the lentiviral vector of the invention or the cell of the invention, and a pharmaceutically acceptable carrier, diluent or excipient.

In one aspect, the invention provides the lentiviral vector of the invention or the cell of the invention for use in therapy.

In one aspect, the invention provides use of the lentiviral vector of the invention or the cell of the invention for the manufacture of a medicament.

In one aspect, the invention provides a method of treatment comprising administering the lentiviral vector of the invention or the cell of the invention to a subject in need thereof.

In one aspect, the invention provides the lentiviral vector of the invention or the cell of the invention for use in treatment or prevention of a hypercholesterolemia.

In another aspect, the invention provides the lentiviral vector of the invention or the cell of the invention for use in treatment or prevention of familial hypercholesterolemia associated conditions, such as atherosclerosis.

In one aspect, the invention provides use of the lentiviral vector of the invention or the cell of the invention for the manufacture of a medicament for treatment or prevention of a hypercholesterolemia.

In one aspect, the invention provides use of the lentiviral vector of the invention or the cell of the invention for the manufacture of a medicament for treatment or prevention of familial hypercholesterolemia associated conditions, such as atherosclerosis. In one aspect, the invention provides a method of treatment or prevention of a hypercholesterolemia comprising administering the lentiviral vector of the invention or the cell of the invention to a subject in need thereof.

In one aspect, the invention provides a method of treatment or prevention of familial hypercholesterolemia associated conditions, such as atherosclerosis, comprising administering the lentiviral vector of the invention or the cell of the invention to a subject in need thereof.

In some embodiments, the hypercholesterolemia is of genetic origin. In some embodiments, the hypercholesterolemia is of non-genetic origin. In some embodiments, a subject having hypercholesterolemia of non-genetic origin has high plasma levels of LDL cholesterol, for example at least 190 or 240 dl/mL.

In one aspect, the invention provides the lentiviral vector of the invention or the cell of the invention for use in treatment or prevention of familial hypercholesterolemia (FH).

In one aspect, the invention provides the lentiviral vector of the invention or the cell of the invention for use in treatment or prevention of familial hypercholesterolemia associated conditions, such as atherosclerosis.

In one aspect, the invention provides use of the lentiviral vector of the invention or the cell of the invention for the manufacture of a medicament for treatment or prevention of familial hypercholesterolemia (FH).

In one aspect, the invention provides use of the lentiviral vector of the invention or the cell of the invention for the manufacture of a medicament for treatment or prevention of familial hypercholesterolemia associated conditions, such as atherosclerosis.

In one aspect, the invention provides a method of treatment or prevention of familial hypercholesterolemia (FH) comprising administering the lentiviral vector of the invention or the cell of the invention to a subject in need thereof.

In one aspect, the invention provides a method of treatment or prevention of familial hypercholesterolemia associated conditions, such as atherosclerosis, comprising administering the lentiviral vector of the invention or the cell of the invention to a subject in need thereof.

In some embodiments, the subject is a human subject.

In some embodiments, the subject is a juvenile. In some embodiments, the subject is a paediatric subject. In some embodiments, the subject is a neonatal subject or an infantile subject.

In some embodiments, the lentiviral vector is administered systemically. In some embodiments, the lentiviral vector is administered by intravenous injection or intraperitoneal injection.

In some embodiments, the lentiviral vector is administered locally. In some embodiments, the lentiviral vector is administered by direct injection, intraarterial injection or intraportal injection.

In some embodiments, the lentiviral vector is administered locally to the liver. In some embodiments, the lentiviral vector is administered by intrahepatic injection, intrahepatic arterial injection or intraportal injection.

In some embodiments, total cholesterol levels are reduced and/or normalised.

In some embodiments, LDL cholesterol levels are reduced and/or normalised.

In some embodiments, the method or treatment further comprises administering an interferon αβ receptor I (IFNARI) inhibitor. In some embodiments, the method or treatment further comprises administering a proteasome inhibitor. In some embodiments, the method or treatment further comprises a subject fasting.

In some embodiments, the method or treatment further comprises administering an interferon αβ receptor I (IFNARI) inhibitor and administering a proteasome inhibitor. In some embodiments, the method or treatment further comprises administering an interferon αβ receptor I (IFNARI) inhibitor and a subject fasting. In some embodiments, the method or treatment further comprises administering a proteasome inhibitor and a subject fasting. In some embodiments, the method or treatment further comprises administering an interferon αβ receptor I (IFNARI) inhibitor, administering a proteasome inhibitor and a subject fasting.

In some embodiments, the IFNARI inhibitor is an anti-IFNARI antibody (e.g. an anti-IFNARI monoclonal antibody). In some embodiments, the IFNARI inhibitor is Marl.

In preferred embodiments, the IFNARI inhibitor is administered before the viral vector. For example, the IFNARI inhibitor may be administered about 1 hour to 24 hours before the viral vector, such as about 1 hour to 12 hours, 1 hour to 6 hours, 1 hour to 5 hours, 1 hour to 4 hours, 2 hours to 24 hours, 2 hours to 12 hours, 2 hours to 6 hours, 2 hours to 5 hours, or 2 hours to 4 hours, preferably about 3 hours before the viral vector.

In some embodiments, the proteasome inhibitor is Bortezomib.

In preferred embodiments, the proteasome inhibitor is administered before the viral vector. For example, the proteasome inhibitor may be administered about 30 minutes to 24 hours before the viral vector, such as about 30 minutes to 12 hours, 30 minutes to 6 hours, 30 minutes to 3 hours, 30 minutes to 2 hours, or 30 minutes to 1.5 hours, preferably about 1 hour before the viral vector.

In preferred embodiments, a subject is fasted (e.g. does not consume food) before the administration of the viral vector. For example, a subject may be fasted for about 6 hours to 72 hours before the administration of the viral vector, such as about 6 hours to 48 hours, 6 hours to 36 hours, 6 hours to 24 hours, 12 hours to 72 hours, 12 hours to 48 hours, 12 hours to 36 hours, or 12 hours to 24 hours, preferably about 24 hours before the administration of the viral vector.

In another aspect, the invention provides a viral vector for use in a method of treatment of a subject, wherein:
(a) the method further comprises administering an interferon αβ receptor I (IFNARI) inhibitor to the subject, optionally before the administration of the viral vector;
(b) the method further comprises administering a proteasome inhibitor to the subject, optionally before the administration of the viral vector; and/or
(c) the method further comprises the subject fasting, optionally before the administration of the viral vector.

In another aspect, the invention provides use of a viral vector for the manufacture of a medicament for treatment of a subject, wherein:
(a) the treatment further comprises administering an interferon αβ receptor I (IFNARI) inhibitor to the subject, optionally before the administration of the viral vector;
(b) the treatment further comprises administering a proteasome inhibitor to the subject, optionally before the administration of the viral vector; and/or
(c) the treatment further comprises the subject fasting, optionally before the administration of the viral vector.

In another aspect, the invention provides a method of treatment comprising administering a viral vector to a subject in need thereof, wherein:
(a) the method further comprises administering an interferon αβ receptor I (IFNARI) inhibitor to the subject, optionally before the administration of the viral vector;
(b) the method further comprises administering a proteasome inhibitor to the subject, optionally before the administration of the viral vector; and/or
(c) the method further comprises the subject fasting, optionally before the administration of the viral vector.

The method may comprise administering one or more dose of the IFNARI inhibitor and/or proteasome inhibitor to the subject, for example one, two, three, or more doses.

The method or treatment may, for example increase transgene (e.g. transgene encoded by the viral vector) output in the subject.

In some embodiments, the method or treatment further comprises administering an interferon αβ receptor I (IFNARI) inhibitor to the subject and administering a proteasome inhibitor to the subject. In some embodiments, the method or treatment further comprises administering an interferon αβ receptor I (IFNARI) inhibitor to the subject and the subject fasting. In some embodiments, the method or treatment further comprises administering a proteasome inhibitor to the subject and the subject fasting. In some embodiments, the method or treatment further comprises administering an interferon αβ receptor I (IFNARI) inhibitor to the subject, administering a proteasome inhibitor to the subject and the subject fasting.

In some embodiments, the treatment is a gene therapy. In some embodiments, the treatment is an in vivo gene therapy. In some embodiments, the viral vector transduces hepatocytes. In some embodiments, the treatment is of familial hypercholesterolemia (FH) or a familial hypercholesterolemia associated condition, such as atherosclerosis. In some embodiments, the treatment is of haemophilia.

In some embodiments, the viral vector is a lentiviral vector.

In preferred embodiments, the viral vector comprises a transgene, such as a nucleotide sequence encoding low density lipoprotein receptor (LDLR), Factor IX (FIX) or Factor VIII (FVIII). In some embodiments, the viral vector comprises a nucleotide sequence encoding low density lipoprotein receptor (LDLR). In some embodiments, the viral vector comprises a nucleotide sequence encoding Factor IX (FIX). In some embodiments, the viral vector comprises a nucleotide sequence encoding Factor VIII (FVIII).

The use of the interferon αβ receptor I (IFNARI) inhibitor, proteasome inhibitor and/or fasting may improve transduction of viral vectors (e.g. lentiviral vectors) with other transgenes, for example the uses may be applied to transgenes other than LDLR, FVIII and FIX, and the methods may also relate to associated diseases.

The transgene may encode a reporter, for example that may be used to monitor (e.g. longitudinally monitor) transduction and/or transgene expression.

In some embodiments, the lentiviral vector is the lentiviral vector of the invention. In some embodiments, the viral vector is a viral vector (e.g. lentiviral vector) as disclosed herein, but lacking the nucleotide sequence encoding the LDLR, or with the nucleotide sequence encoding the LDLR replaced by a different nucleotide sequence (e.g. transgene).

In some embodiments, the transgene is operably linked to one or more miRNA target sequence. The miRNA target sequence may be as described herein. In some embodiments, the one or more miRNA target sequence suppresses transgene expression in one or more cell type other than hepatocytes.

In some embodiments, the viral vector is a CD47^{high} viral vector. In some embodiments, the viral vector is a MHC-I^{free} viral vector. In some embodiments, the viral vector is a CD47^{high}/MHC-I^{free} viral vector.

In some embodiments, the IFNARI inhibitor is an anti-IFNARI antibody (e.g. an anti-IFNARI monoclonal antibody). In some embodiments, the IFNARI inhibitor is Marl.

In preferred embodiments, the IFNARI inhibitor is administered to the subject before the viral vector. For example, the IFNARI inhibitor may be administered to the subject about 1 hour to 24 hours before the viral vector, such as about 1 hour to 12 hours, 1 hour to 6 hours, 1 hour to 5 hours, 1 hour to 4 hours, 2 hours to 24 hours, 2 hours to 12 hours, 2 hours to 6 hours, 2 hours to 5 hours, or 2 hours to 4 hours, preferably about 3 hours before the viral vector.

In some embodiments, the proteasome inhibitor is Bortezomib.

In preferred embodiments, the proteasome inhibitor is administered to the subject before the viral vector. For example, the proteasome inhibitor may be administered to the subject about 30 minutes to 24 hours before the viral vector, such as about 30 minutes to 12 hours, 30 minutes to 6 hours, 30 minutes to 3 hours, 30 minutes to 2 hours, or 30 minutes to 1.5 hours, preferably about 1 hour before the viral vector.

In preferred embodiments, the subject is fasted (e.g. does not consume food) before the administration of the viral vector. For example, the subject may be fasted for about 6 hours to 72 hours before the administration of the viral vector, such as about 6 hours to 48 hours, 6 hours to 36 hours, 6 hours to 24 hours, 12 hours to 72 hours, 12 hours to 48 hours, 12 hours to 36 hours, or 12 hours to 24 hours, preferably about 24 hours before the administration of the viral vector.

The method may increase vector copy number in heptatocytes and/or LSECs.

### DESCRIPTION OF THE DRAWINGS

FIG. 1

Generation of LV encoding Ldlr. (a) Single values and mean with standard error of the mean (SEM) of infectious titer (left panel), LV particles (ng HIV Gag p24/mL, middle panel) and specific infectivity (TU/ng p24) of non-concentrated LV encoding hLDLR (full black dots), compared to LV encoding GFP (empty black dots), produced through transient transfection. (b) Schematic of the third generation lentiviral vectors (LV genome transfer plasmid). CMV, cytomegalovirus promoter; LTRs, long terminal repeats: the 3′ LTR has an almost complete deletion of the U3 region (AU3); ψ, the packaging sequence; RRE, Rev response element; cPPT, central polypurine tract; pA, polyadenylation signal; ET, enhanced transthyretin promoter; WPRE, posttranscriptional element from the genome of the woodchuck hepatitis virus. (c) Single values and mean with SEM of infectious titer (left panel), LV particles (ng HIV Gag p24/mL, middle panel) and specific infectivity (TU/ng p24, right panel) of non-concentrated antisense-oriented LV encoding hLDLR (full black dots), compared to anti-sense-oriented LV encoding hFIX (empty black dots), pseudotyped with VSV.G. (d) Single values and mean with SEM of percentage of hLDLR positive Huh7 cells (left part of the panel), mean fluorescence intensity (MFI) (middle part of the panel) and VCN (right part of the panel) of Huh7 cells transduced in triplicate with indicated LV at indicated multiplicity of infection (MOI). (e) Same as (d) but concerning Hepa1.6 cells.

FIG. 2

Test of liver gene transfer efficiency in Ldlr^{-/-} adult mice. (a) Experimental design: 8-wk old mice, C57 or Ldlr^{-/-} are injected i.v. with LV (dose 3.5×10¹⁰ TU/Kg), pseudotyped with VSV.G, encoding hFIX reporter transgene, to compare transgene output over time and transduction efficiency at end of experiment; (b) Mean with SEM of hFIX antigen measured in the plasma of LV treated Ldlr^{-/-} or C57 mice over time (n=5 for C57 mice, n=5 for Ldlr^{-/-} mice). Two-way ANOVA with Sidak's multiple comparisons test. Single values and mean with SEM of VCN measured in FACS-sorted liver subpopulations: (c) hepatocytes, (d) liver sinusoidal endothelial cells (LSEC) (e) Kupffer cells (KC), 3 months after LV administration.

FIG. 3

LDLR liver gene transfer in Ldlr^{-/-} juvenile mice. (a) Experimental design: 2 weeks old mice, Ldlr^{-/-}, are treated with LV, pseudotyped with VSV.G, encoding mLDLR transgene reverse oriented, and bled to assess normalization of total and LDL cholesterol. Starting from week 7 after LV, some of the mice are challenged with WD for 3 months. Total and LDL cholesterol are monitored during challenge. Mice are then moved back to standard diet for one additional month, before being killed for histopathological analysis. Mean with SEM of total and LDL circulating cholesterol measured in the serum of experimental mice during the first weeks following LV administration (b, c) and throughout the whole experiment (d, e).

FIG. 4

Histopathology Analysis of Atherosclerosis in Ldlr^{-/-} Mice Treated with LV

(a) Table with percentage of atherosclerosis observed in the different groups; (b) hematoxylin and eosin staining of aortic sinus of Ldlr^{-/-} mice, C57 or Ldlr^{-/-} LV treated challenged with western diet, magnification 500 μm or (c) 1000 μm; (d) hematoxylin and eosin staining of aortic arch and (e) thoracic aorta of Ldlr^{-/-} mice, C57 or Ldlr^{-/-} LV treated, challenged with western diet, magnification 500 μm.

FIG. 5

LDLR gene transfer in young Ldlr^{-/-} mice: 1 year follow up. (a) Experimental design: 2 weeks old mice, Ldlr^{-/-}, are treated with LV, pseudotyped with VSV.G, encoding mLDLR reverse oriented (4×10¹⁰ TU/Kg), monitored overtime for cholesterol and LDL, and killed 1 year post LV. Total (b) and LDL (c) cholesterol measured in the serum of LV treated or control mice. Two-way ANOVA with Tukey's multiple comparisons test.

FIG. 6

Expression of LDLR inhibitor during LV production rescues productivity of sense-oriented LV, which fully normalize LDL-C in Ldlr^{-/-} mice. (a) Single values and mean with SEM of infectious titer of non-concentrated hLDLR LV, produced through transient transfection with (empty black dots) or without (full black dots) the addition of LDLR non-secreted inhibitor (PCSK9 S127R). (b) Schematic representation of in vivo experimental design: hLDLR i.v. injection in juvenile mice (dose: 4-6×10¹⁰ TU/Kg), followed over time. (c) Total and LDL (d) circulating cholesterol in the serum of experimental mice over time. Two-way ANOVA with Tukey's multiple comparisons test.

FIG. 7

Bortezomib and Anti-IFNAR1, as single treatments, enhance LV gene therapy potency. (a) Experimental design: 8 weeks old Alb-F8*R593C mice are injected i.v. with Bortezomib (1 mg/kg) or PBS 1 hour before receiving c.o.hFVIII LV i.v., dose: 4.5×101⁰TU/Kg. Then, hFVIII is monitored in treated mice longitudinally. (b) Mean with SEM of hFVIII antigen measured in the plasma of LV treated mice overtime (n=4 per each group). Two-way ANOVA. (c) Single values and mean with SEM of VCN measured in fractioned and FACS-sorted liver subpopulations: nPC, Hep, LSEC, KC, pDC of experimental mice, total liver of experimental mice, pre-treated with Marl (red dots) or with PBS (controls, black dots). Mann-Whitney test. (d) Experimental design: 8 weeks old Alb-F8*R593C mice are injected i.v. with anti-IFNAR1 (1 mg/mouse) or PBS 3 hour before receiving c.o.hFVIII LV i.v., dose: 7×10¹⁰. Then, hFVIII is monitored in treated mice longitudinally. (e) Mean with SEM of hFVIII antigen measured in the plasma of LV treated mice over time (n=5-6 per each group). Two-way ANOVA. (f) Single values and mean with SEM of VCN measured in fractioned and FACS-sorted liver subpopulations: nPC, Hep, LSEC, KC, pDC of experimental mice, pre-treated with anti-IFNAR1 (red dots) or with PBS (controls, black dots), 5 months post LV. Mann-Whitney test.

FIG. 8

(a) Experimental design: 8 weeks old mice are injected or not with Marl (anti-IFNAR1) 3 hours before and/or Bortezomib 1 hour before receiving hFIX-IRES-GFP LV i.v. Then, hFIX is monitored in treated mice longitudinally. (b) Mean with SEM of hFIX antigen measured in the plasma of LV treated mice over time (n=5 per each group). Two-way ANOVA with Dunnett's multiple comparisons test vs control. Single values and mean with SEM of VCN measured in fractionated and FACS-sorted liver subpopulations: hepatocytes (c), Kupffer cells (d) liver endothelial cells (e), plasmacytoid dendritic cells (f) and total liver (g) of treated mice. Kruskal-Wallis test with Dunn's multiple comparisons test.

FIG. 9

Fasting increases LV gene transfer to hepatocytes. (a) Experimental design: 8 weeks old mice are fasted 24 hours. Then, they are injected i.v. with hFIX-IRES-GFP LV, together with 8 weeks old mice non-fasted. Their circulating hFIX amounts are monitored longitudinally. (b) Mean with SEM of hFIX antigen measured in the plasma of LV treated mice overtime (n=4 per each group). Two-way ANOVA. (c) Single values and mean with SEM of percentage of liver area expressing GFP by immunofluorescence analysis 3 months after LV administration, Mann-Whitney test. (d) Single values and mean with SEM of expression analysis by quantitative PCR of WPRE normalized on the endogenous hprt gene on RNA extracted from the livers of treated mice. Mann-Whitney test. (e) Single values and mean with SEM of VCN measured in total liver and spleen of LV treated mice. Mann-Whitney test.

FIG. 10

Monitoring LDLR over fasting. (a) Experimental design: 8 weeks old mice are fasted 12 or 24 hours and re-fed for additional 24 hours. LDLR expression is monitored overtime. (b) Single values and mean with SEM of expression analysis by quantitative PCR of Ldlr, normalized on the endogenous hprt gene on RNA extracted from the livers of C57 or Ldlr^{-/-} mice. Kruskal-Wallis test with multiple comparisons over control group.

FIG. 11

LV transduction enhancers additive effect in C57 and Ldlr^{-/-} mice. (a) Experimental design: 8 weeks old mice C57 or Ldlr^{-/-} (n=4-6 per each condition) are injected with Bortezomib and/or Marl and/or fasted for 24 hours, before receiving LV (dose 2.75×10¹⁰ TU/Kg) i.v. Then, hFIX is monitored in treated mice longitudinally. (b, c) Mean with SEM of hFIX antigen measured in the plasma of LV treated mice over time (4 and 26 wk post LV timepoint). Two-way ANOVA with Dunnett's multiple comparisons test vs LV control, performed for each of the two strains used.

### DETAILED DESCRIPTION OF THE INVENTION

The terms “comprising”, “comprises” and “comprised of” as used herein are synonymous with “including” or “includes”; or “containing” or “contains”, and are inclusive or open-ended and do not exclude additional, non-recited members, elements or steps. The terms “comprising”, “comprises” and “comprised of” also include the term “consisting of”.

### Low Density Lipoprotein Receptor (LDLR)

In one aspect, the invention provides a lentiviral vector comprising a nucleotide sequence encoding low density lipoprotein receptor (LDLR) or a fragment thereof.

The low density lipoprotein (LDL) receptor (LDLR or LDL-R) is a protein that mediates the endocytosis of cholesterol-rich LDL. LDLR binds LDL, which is the major cholesterol-carrying lipoprotein of plasma, and transports it into cells by endocytosis.

The LDLR may be, for example, human LDLR or mouse LDLR, preferably human LDLR.

Example LDLR amino acid sequences include:

(SEQ ID NO: 1; human LDLR)
MGPWGWKLRWTVALLLAAAGTAVGDRCERNEFQCQDGKCISYKWVCDGSAECQDGSDESQET
CLSVTCKSGDFSCGGRVNRCIPQFWRCDGQVDCDNGSDEQGCPPKTCSQDEFRCHDGKCISR
QFVCDSDRDCLDGSDEASCPVLTCGPASFQCNSSTCIPQLWACDNDPDCEDGSDEWPQRCRG
LYVFQGDSSPCSAFEFHCLSGECIHSSWRCDGGPDCKDKSDEENCAVATCRPDEFQCSDGNC
IHGSRQCDREYDCKDMSDEVGCVNVTLCEGPNKFKCHSGECITLDKVCNMARDCRDWSDEPI
KECGTNECLDNNGGCSHVCNDLKIGYECLCPDGFQLVAQRRCEDIDECQDPDTCSQLCVNLE
GGYKCQCEEGFQLDPHTKACKAVGSIAYLFFTNRHEVRKMTLDRSEYTSLIPNLRNVVALDT
EVASNRIYWSDLSQRMICSTQLDRAHGVSSYDTVISRDIQAPDGLAVDWIHSNIYWTDSVLG
TVSVADTKGVKRKTLFRENGSKPRAIVVDPVHGFMYWTDWGTPAKIKKGGLNGVDIYSLVTE
NIQWPNGITLDLLSGRLYWVDSKLHSISSIDVNGGNRKTILEDEKRLAHPFSLAVFEDKVFW
TDIINEAIFSANRLTGSDVNLLAENLLSPEDMVLFHNLTQPRGVNWCERTTLSNGGCQYLCL
PAPQINPHSPKFTCACPDGMLLARDMRSCLTEAEAAVATQETSTVRLKVSSTAVRTQHTTTR
PVPDTSRLPGATPGLTTVEIVTMSHQALGDVAGRGNEKKPSSVRALSIVLPIVLLVFLCLGV
FLLWKNWRLKNINSINFDNPVYQKTTEDEVHICHNQDGYSYPSRQMVSLEDDVA
(SEQ ID NO: 38; mouse LDLR)
MSTADLMRRWVIALLLAAAGVAAEDSCSRNEFQCRDGKCIASKWVCDGSPECPDGSDESPET
CMSVTCQSNQFSCGGRVSRCIPDSWRCDGQVDCENDSDEQGCPPKTCSQDDFRCQDGKCISP
QFVCDGDRDCLDGSDEAHCQATTCGPAHFRCNSSICIPSLWACDGDVDCVDGSDEWPQNCQG
RDTASKGVSSPCSSLEFHCGSSECIHRSWVCDGEADCKDKSDEEHCAVATCRPDEFQCADGS
CIHGSRQCDREHDCKDMSDELGCVNVTQCDGPNKFKCHSGECISLDKVCDSARDCQDWSDEP
IKECKTNECLDNNGGCSHICKDLKIGSECLCPSGFRLVDLHRCEDIDECQEPDTCSQLCVNL
EGSYKCECQAGFHMDPHTRVCKAVGSIGYLLFTNRHEVRKMTLDRSEYTSLLPNLKNVVALD
TEVINNRIYWSDLSQKKIYSALMDQAPNLSYDTIISEDLHAPDGLAVDWIHRNIYWTDSVPG
SVSVADTKGVKRRTLFQEAGSRPRAIVVDPVHGFMYWTDWGTPAKIKKGGLNGVDIHSLVTE
NIQWPNGITLDLSSGRLYWVDSKLHSISSIDVNGGNRKTILEDENRLAHPESLAIYEDKVYW
TDVINEAIFSANRLTGSDVNLVAENLLSPEDIVLFHKVTQPRGVNWCETTALLPNGGCQYLC
LPAPQIGPHSPKFTCACPDGMLLAKDMRSCLTEVDTVLTTQGTSAVRPVVTASATRPPKHSE
DLSAPSTPRQPVDTPGLSTVASVTVSHQVQGDMAGRGNEEQPHGMRFLSIFFPIALVALLVL
GAVLLWRNWRLKNINSINFDNPVYQKTTEDELHICRSQDGYTYPSRQMVSLEDDVA

In some embodiments, the LDLR comprises or consists of an amino acid sequence that has at least 70%, at least 80%, at least 90%, at least 95%, at least 96%, at least 97%, at least 98% or at least 99% sequence identity to SEQ ID NO: 1 or 38, preferably SEQ ID NO: 1, or a fragment thereof.

In some embodiments, the LDLR comprises or consists of an amino acid sequence that has at least 70% sequence identity to SEQ ID NO: 1 or 38, preferably SEQ ID NO: 1, or a fragment thereof.

In some embodiments, the LDLR comprises or consists of the amino acid sequence of SEQ ID NO: 1 or 38, preferably SEQ ID NO: 1, or a fragment thereof.

Example nucleotide sequences encoding LDLR include:

(SEQ ID NO: 2; human LDLR)
ATGGGGCCCTGGGGCTGGAAATTGCGCTGGACCGTCGCCTTGCTCCTCGCCGCGGCGGGGAC
TGCAGTGGGCGACAGATGCGAAAGAAACGAGTTCCAGTGCCAAGACGGGAAATGCATCTCCT
ACAAGTGGGTCTGCGATGGCAGCGCTGAGTGCCAGGATGGCTCTGATGAGTCCCAGGAGACG
TGCTTGTCTGTCACCTGCAAATCCGGGGACTTCAGCTGTGGGGGCCGTGTCAACCGCTGCAT
TCCTCAGTTCTGGAGGTGCGATGGCCAAGTGGACTGCGACAACGGCTCAGACGAGCAAGGCT
GTCCCCCCAAGACGTGCTCCCAGGACGAGTTTCGCTGCCACGATGGGAAGTGCATCTCTCGG
CAGTTCGTCTGTGACTCAGACCGGGACTGCTTGGACGGCTCAGACGAGGCCTCCTGCCCGGT
GCTCACCTGTGGTCCCGCCAGCTTCCAGTGCAACAGCTCCACCTGCATCCCCCAGCTGTGGG
CCTGCGACAACGACCCCGACTGCGAAGATGGCTCGGATGAGTGGCCGCAGCGCTGTAGGGGT
CTTTACGTGTTCCAAGGGGACAGTAGCCCCTGCTCGGCCTTCGAGTTCCACTGCCTAAGTGG
CGAGTGCATCCACTCCAGCTGGCGCTGTGATGGTGGCCCCGACTGCAAGGACAAATCTGACG
AGGAAAACTGCGCTGTGGCCACCTGTCGCCCTGACGAATTCCAGTGCTCTGATGGAAACTGC
ATCCATGGCAGCCGGCAGTGTGACCGGGAATATGACTGCAAGGACATGAGCGATGAAGTTGG
CTGCGTTAATGTGACACTCTGCGAGGGACCCAACAAGTTCAAGTGTCACAGCGGCGAATGCA
TCACCCTGGACAAAGTCTGCAACATGGCTAGAGACTGCCGGGACTGGTCAGATGAACCCATC
AAAGAGTGCGGGACCAACGAATGCTTGGACAACAACGGCGGCTGTTCCCACGTCTGCAATGA
CCTTAAGATCGGCTACGAGTGCCTGTGCCCCGACGGCTTCCAGCTGGTGGCCCAGCGAAGAT
GCGAAGATATCGATGAGTGTCAGGATCCCGACACCTGCAGCCAGCTCTGCGTGAACCTGGAG
GGTGGCTACAAGTGCCAGTGTGAGGAAGGCTTCCAGCTGGACCCCCACACGAAGGCCTGCAA
GGCTGTGGGCTCCATCGCCTACCTCTTCTTCACCAACCGGCACGAGGTCAGGAAGATGACGC
TGGACCGGAGCGAGTACACCAGCCTCATCCCCAACCTGAGGAACGTGGTCGCTCTGGACACG
GAGGTGGCCAGCAATAGAATCTACTGGTCTGACCTGTCCCAGAGAATGATCTGCAGCACCCA
GCTTGACAGAGCCCACGGCGTCTCTTCCTATGACACCGTCATCAGCAGAGACATCCAGGCCC
CCGACGGGCTGGCTGTGGACTGGATCCACAGCAACATCTACTGGACCGACTCTGTCCTGGGC
ACTGTCTCTGTTGCGGATACCAAGGGCGTGAAGAGGAAAACGTTATTCAGGGAGAACGGCTC
CAAGCCAAGGGCCATCGTGGTGGATCCTGTTCATGGCTTCATGTACTGGACTGACTGGGGAA
CTCCCGCCAAGATCAAGAAAGGGGGCCTGAATGGTGTGGACATCTACTCGCTGGTGACTGAA
AACATTCAGTGGCCCAATGGCATCACCCTAGATCTCCTCAGTGGCCGCCTCTACTGGGTTGA
CTCCAAACTTCACTCCATCTCAAGCATCGATGTCAACGGGGGCAACCGGAAGACCATCTTGG
AGGATGAAAAGAGGCTGGCCCACCCCTTCTCCTTGGCCGTCTTTGAGGACAAAGTATTTTGG
ACAGATATCATCAACGAAGCCATTTTCAGTGCCAACCGCCTCACAGGTTCCGATGTCAACTT
GTTGGCTGAAAACCTACTGTCCCCAGAGGATATGGTTCTCTTCCACAACCTCACCCAGCCAA
GAGGAGTGAACTGGTGTGAGAGGACCACCCTGAGCAATGGCGGCTGCCAGTATCTGTGCCTC
CCTGCCCCGCAGATCAACCCCCACTCGCCCAAGTTTACCTGCGCCTGCCCGGACGGCATGCT
GCTGGCCAGGGACATGAGGAGCTGCCTCACAGAGGCTGAGGCTGCAGTGGCCACCCAGGAGA
CATCCACCGTCAGGCTAAAGGTCAGCTCCACAGCCGTAAGGACACAGCACACAACCACCCGA
CCTGTTCCCGACACCTCCCGGCTGCCTGGGGCCACCCCTGGGCTCACCACGGTGGAGATAGT
GACAATGTCTCACCAAGCTCTGGGCGACGTTGCTGGCAGAGGAAATGAGAAGAAGCCCAGTA
GCGTGAGGGCTCTGTCCATTGTCCTCCCCATCGTGCTCCTCGTCTTCCTTTGCCTGGGGGTC
TTCCTTCTATGGAAGAACTGGCGGCTTAAGAACATCAACAGCATCAACTTTGACAACCCCGT
CTATCAGAAGACCACAGAGGATGAGGTCCACATTTGCCACAACCAGGACGGCTACAGCTACC
CCTCGAGACAGATGGTCAGTCTGGAGGATGACGTGGCGTGA
(SEQ ID NO: 39; mouse LDLR)
ATGAGCACCGCGGATCTGATGCGTCGCTGGGTCATCGCCCTGCTCCTGGCTGCTGCCGGAGT
TGCAGCAGAAGACTCATGCAGCAGGAACGAGTTCCAGTGTAGAGACGGAAAATGCATCGCTA
GCAAGTGGGTGTGCGATGGCAGCCCCGAGTGCCCGGATGGCTCCGATGAGTCCCCAGAGACA
TGCATGTCTGTCACCTGTCAGTCCAATCAATTCAGCTGTGGAGGCCGTGTCAGCCGATGCAT
TCCTGACTCCTGGAGATGTGATGGACAGGTAGACTGTGAAAATGACTCAGACGAACAAGGCT
GTCCCCCCAAGACGTGCTCCCAGGATGACTTCCGATGCCAGGATGGCAAGTGCATCTCCCCG
CAGTTTGTGTGTGATGGAGACCGAGATTGCCTAGATGGCTCTGATGAGGCCCACTGCCAGGC
CACCACTTGTGGCCCCGCCCACTTCCGCTGCAACTCATCCATATGCATCCCCAGTCTTTGGG
CCTGCGACGGGGATGTCGACTGTGTTGACGGCTCCGATGAGTGGCCACAGAACTGCCAGGGC
CGAGACACGGCCTCCAAAGGCGTTAGCAGCCCCTGCTCCTCCCTGGAGTTCCACTGTGGTAG
CAGTGAGTGTATCCATCGCAGCTGGGTCTGTGACGGCGAGGCAGACTGCAAGGACAAGTCAG
ATGAGGAGCACTGCGCGGTGGCCACCTGCCGACCTGATGAATTCCAGTGTGCAGATGGCTCC
TGCATTCACGGTAGCCGCCAGTGTGACCGTGAACATGACTGCAAGGACATGAGCGACGAGCT
CGGCTGCGTCAATGTGACACAGTGTGATGGCCCCAACAAGTTCAAGTGTCACAGTGGGGAGT
GCATCAGCTTGGACAAGGTGTGCGACTCCGCCCGCGACTGCCAGGACTGGTCGGATGAGCCC
ATCAAGGAGTGCAAGACCAACGAGTGTTTGGACAACAATGGTGGCTGTTCCCACATCTGCAA
GGACCTCAAGATTGGCTCTGAGTGCCTGTGTCCCAGCGGCTTCCGGTTGGTGGACCTCCACA
GGTGTGAAGATATTGACGAGTGTCAGGAGCCAGACACCTGCAGCCAGCTCTGTGTGAACCTG
GAAGGCAGCTACAAGTGTGAGTGCCAGGCCGGCTTCCACATGGACCCACACACCAGGGTCTG
CAAGGCTGTGGGCTCCATAGGCTATCTGCTCTTCACCAACCGCCACGAGGTCCGGAAGATGA
CCCTGGACCGCAGCGAGTACACCAGTCTGCTCCCCAACCTGAAGAATGTGGTGGCTCTCGAC
ACGGAGGTGACCAACAATAGAATCTACTGGTCCGACCTGTCCCAAAAAAAGATCTACAGCGC
CCTGATGGACCAGGCCCCTAACTTGTCCTACGACACCATCATCAGTGAGGACCTGCATGCCC
CTGACGGGCTGGCGGTAGACTGGATCCACCGCAACATCTACTGGACAGATTCAGTCCCAGGC
AGCGTATCTGTGGCTGACACCAAGGGCGTAAAGAGGAGGACACTGTTCCAAGAGGCAGGGTC
CAGACCCAGAGCCATCGTAGTGGACCCTGTGCATGGCTTCATGTACTGGACAGATTGGGGAA
CACCCGCCAAGATCAAGAAAGGGGGTTTGAATGGTGTGGACATCCACTCACTGGTGACCGAA
AACATCCAGTGGCCAAATGGCATCACACTAGATCTTTCCAGTGGCCGTCTCTATTGGGTTGA
TTCCAAACTCCACTCTATCTCCAGCATCGATGTCAATGGGGGCAATCGGAAAACCATTTTGG
AGGATGAGAACCGGCTGGCCCACCCCTTCTCCTTGGCCATCTATGAGGACAAAGTGTATTGG
ACAGATGTCATAAACGAAGCCATTTTCAGTGCCAATCGACTCACGGGTTCAGATGTGAATTT
GGTGGCTGAAAACCTCTTGTCCCCGGAGGACATTGTCCTGTTCCACAAGGTCACACAGCCTA
GAGGGGTGAACTGGTGTGAGACAACAGCCCTCCTCCCCAATGGTGGTTGCCAGTACCTGTGC
CTGCCCGCCCCACAGATCGGTCCCCACTCGCCCAAATTCACCTGCGCCTGCCCTGATGGCAT
GCTGCTGGCCAAGGACATGCGGAGCTGCCTCACAGAAGTCGACACTGTACTGACCACCCAGG
GGACATCCGCCGTCCGGCCTGTGGTCACCGCATCAGCTACCAGGCCACCGAAGCACAGTGAG
GATCTCTCAGCTCCCAGTACTCCTAGGCAGCCTGTGGACACCCCAGGGCTCAGCACAGTGGC
GTCAGTGACAGTGTCCCACCAAGTCCAGGGTGACATGGCTGGCAGAGGGAATGAGGAGCAGC
CACATGGTATGAGGTTCCTGTCCATCTTCTTCCCTATTGCACTGGTTGCCCTCCTTGTCCTT
GGGGCCGTCCTGCTGTGGAGGAACTGGCGGCTGAAGAACATCAACAGCATAAACTTTGACAA
CCCAGTCTACCAGAAGACCACAGAGGACGAGCTCCACATTTGCCGAAGCCAGGATGGCTATA
CCTACCCCTCAAGACAGATGGTCAGCCTGGAGGACGATGTGGCATGA

In some embodiments, the nucleotide sequence encoding LDLR comprises or consists of a nucleotide sequence that has at least 70%, at least 80%, at least 90%, at least 95%, at least 96%, at least 97%, at least 98% or at least 99% sequence identity to SEQ ID NO: 2 or 39, preferably SEQ ID NO: 2, or a fragment thereof.

In some embodiments, the nucleotide sequence encoding LDLR comprises or consists of a nucleotide sequence that has at least 70% sequence identity to SEQ ID NO: 2 or 39, preferably SEQ ID NO: 2, or a fragment thereof.

In some embodiments, the nucleotide sequence encoding LDLR comprises or consists of the nucleotide sequence of SEQ ID NO: 2 or 39, preferably SEQ ID NO: 2, or a fragment thereof.

A fragment and/or variant of LDLR may retain LDLR activity (e.g. the activity of SEQ ID NO: 1 or 38). For example, a fragment and/or variant of LDLR may bind LDL and transports it into cells by endocytosis. Suitably, a fragment and/or variant of LDLR may have the same or similar activity to LDLR, for example may have at least 40%, at least 50%, at least 60%, at least 70%, at least 80%, at least 90%, at least 95% or 100% of the activity of LDLR (e.g. the LDLR of SEQ ID NO: 1 or 38). The skilled person would be able to generate fragments and/or variants, for example using conservative substitutions, based on the known structural and functional features of LDLR.

Lentiviral vector In one aspect, the invention provides a lentiviral vector comprising a nucleotide sequence encoding low density lipoprotein receptor (LDLR).

A “lentiviral vector” may be in the form of a lentiviral particle. In other embodiments, a “lentiviral vector” may comprise a lentiviral genome, optionally wherein the lentiviral genome is enveloped. As used herein, a “lentiviral genome” may refer to a genome that comprises at least one element derived or derivable from a lentivirus genome.

Lentivirus is a genus of retroviruses, which contain an RNA genome that is converted to DNA in the transduced cell by a reverse transcriptase. Lentiviral vectors can transduce a wide range of cell types and integrate into the host genome in both dividing and post-mitotic cells, resulting in long-term expression of the protein-coding sequence both in vitro and in vivo.

The basic genes required for lentivirus survival and function are the gag, pol, and env genes: gag encodes structural proteins; pol encodes enzymes required for reverse transcription and integration into the host cell genome; and env encodes the viral envelope glycoprotein. Lentiviruses may also have additional cis-acting elements, such as a rev response element (RRE), which enables the efficient export of RNA transcripts of the integrated provirus from the nucleus to the cytoplasm of an infected target cell; a retroviral psi packaging element, which is involved in regulating the essential process of packaging the retroviral RNA genome into the viral capsid during replication; a primer binding site (PBS), where reverse transcription is initiated; the TAT activation region (TAR); splice donor and acceptor sites; and central and terminal polypurine tracts, which allow initiation of plus-strand synthesis.

In a lentivirus genome, the elements are typically flanked at both ends by regions called long terminal repeats (LTRs). The LTRs are responsible for integration and transcription. LTRs may also serve as enhancer-promoter sequences and can control the expression of the lentiviral genes. The LTRs themselves are identical or near-identical sequences that can typically be divided into three regions: U3, R and U5. LTRs may be naturally occurring or may be modified. For example, U3 and U5 modifications are described in Iwakuma et al. (1999) Virology 261: 120-132.

The lentiviral vector of the present invention may comprise a minimal lentiviral genome. As used herein, a “minimal lentiviral genome” may mean that the lentiviral genome has been manipulated so as to remove the non-essential elements and to retain the essential elements in order to provide the required functionality to infect, transduce and deliver a nucleotide sequence of interest to a target host cell (see, for example, Kim et al. (1998) Journal of Virology 72: 811-816; Sertkaya et al. (2021) Scientific Reports 11: 1-15).

A lentiviral genome may comprise from 5′ to 3′: a 5′ LTR, one or more lentiviral-derived cis-acting elements, and a 3′ LTR. Suitably, a lentiviral genome may comprise from 5′ to 3′: a 5′ LTR, a RRE, and a 3′ LTR. Suitably, a lentiviral genome may comprise from 5′ to 3′: a 5′ LTR, a retroviral psi packaging element, a RRE, and a 3′ LTR. Suitably, a lentiviral genome may comprise from 5′ to 3′: a 5′ LTR, a retroviral psi packaging element, a RRE, a cPPT, and a 3′ LTR. Suitably, a lentiviral genome may comprise from 5′ to 3′: a 5′ LTR, a PBS, a retroviral psi packaging element, a RRE, a cPPT, and a 3′ LTR.

A lentiviral genome may further comprise a protein-coding sequence and, optionally, one or more regulatory elements (e.g. operably linked to the protein-coding sequence). Suitably, a lentiviral genome may comprise from 5′ to 3′: a 5′ LTR, a RRE, a protein-coding sequence, and a 3′ LTR. Suitably, a lentiviral genome may comprise from 5′ to 3′: a 5′ LTR, a retroviral psi packaging element, a RRE, a protein-coding sequence, and a 3′ LTR. Suitably, a lentiviral genome may comprise from 5′ to 3′: a 5′ LTR, a retroviral psi packaging element, a RRE, a cPPT, a protein-coding sequence, and a 3′ LTR. Suitably, lentiviral genome may comprise from 5′ to 3′: a 5′ LTR, a PBS, a retroviral psi packaging element, a RRE, a cPPT, a protein-coding sequence, and a 3′ LTR. Preferably, the protein-coding sequence is in reverse orientation.

The lentiviral vector of the present invention may be replication-defective. Typically, at least part of one or more protein coding regions essential for replication may be removed from the lentiviral genome. This makes the lentiviral vector “replication-defective” or “replication-incompetent”. Suitably, one or more of gag, pol, rev, and env genes are deleted (at least partially) in a replication-defective lentiviral vector. Suitably, each of the gag, pol, rev, and env genes are deleted (at least partially) in a replication-defective lentiviral vector. Optionally, the lentiviral vector lacks a functional gag-pol and/or env gene and/or other genes essential for replication.

The lentiviral vector of the present invention may be derived from any lentivirus. As used herein “lentivirus-derived” or “lentivirus-based” may mean that the lentiviral genome comprises one or more elements from said lentivirus. For example, the coding regions of viral proteins may be deleted, but one or more cis-acting element may be retained from said lentivirus.

The lentiviral vector may be derived from a primate lentivirus. Examples of “primate” lentiviruses include, but are not limited to, human immunodeficiency virus (HIV) and simian immunodeficiency virus (SIV). The lentiviral vector may be derived from a non-primate lentivirus (i.e. derived from a lentivirus which does not primarily infect primates, especially humans). Examples of “non-primate” lentiviruses include, but are not limited to, the prototype “slow virus” visna/maedi virus (VMV), caprine arthritis-encephalitis virus (CAEV), equine infectious anaemia virus (EIAV), feline immunodeficiency virus (FIV), and bovine immunodeficiency virus (BlV).

Suitably, the lentiviral vector of the present invention is a HIV-derived lentiviral vector. As used herein “HIV-derived” or “HIV-based” may mean that the lentiviral genome comprises one or more element from HIV. For example, the coding regions of HIV viral proteins may be deleted, and one or more HIV cis-acting element may retained in the lentiviral genome (see, for example, Johnson (2021) Molecular Therapy-Methods & Clinical Development 21: 451-465). A HIV-derived lentiviral genome may comprise from 5′ to 3′: a 5′ LTR, one or more HIV-derived cis-acting elements (e.g. RRE and/or cPPT), and a 3′ LTR.

Suitably, the lentiviral vector of the present invention is a HIV-1-derived lentiviral vector. The prototype lentiviral vector system is based on HIV-1 (see, for example, Merten et al. (2016) Molecular Therapy-Methods & Clinical Development 3: 16017). It has been shown that sequences that extend into the gag open reading frame may be important for packaging of HIV-1. Therefore, HIV-1 vectors often contain the relevant portion of gag in which the translational initiation codon has been mutated. In addition, HIV-1 vectors often also contain a portion of the env gene that includes the RRE. Rev binds to RRE, which permits the transport of full-length or singly spliced mRNAs from the nucleus to the cytoplasm. In the absence of rev and/or a RRE, full-length HIV-1 RNAs may accumulate in the nucleus. Alternatively, a constitutive transport element from certain simple retroviruses such as Mason-Pfizer monkey virus can be used to relieve the requirement for rev and a RRE. A HIV-1-derived lentiviral genome may comprise from 5′ to 3′: a 5′ LTR, one or more HIV-1-derived cis-acting elements (e.g. a PBS, a retroviral psi packaging element, a RRE and/or a cPPT), and a 3′ LTR.

The lentiviral vector of the present invention may be a self-inactivating lentiviral vector. As used herein, “self-inactivating” or “SIN” lentiviral vectors may comprise lentiviral genomes in which the lentiviral enhancer and promoter sequences have been deleted (see, for example, Zufferey (1998) Journal of Virology 72: 9873-9880; Miyoshi et al. (1998) Journal of Virology 72: 8150-8157). SIN lentiviral vectors can be generated and transduce non-dividing cells in vivo with an efficacy similar to that of wild-type vectors. The transcriptional inactivation of the long terminal repeat (LTR) in the SIN provirus can prevent mobilisation by replication-competent virus. This can also enable the regulated expression of genes from internal promoters by eliminating any cis-acting effects of the LTR.

The lentiviral vector of the present invention may be integration competent. As used herein, an “integration competent” lentiviral vector is capable of integrating into the genome of a host cell. In contrast to integration competent lentiviral vectors, integration defective lentiviral vectors (IDLVs) can be produced, for example by packaging the lentiviral vector with catalytically inactive integrase (such as an HIV integrase bearing the D64V mutation in the catalytic site) or by modifying or deleting essential att sequences from the lentiviral genome LTR, or by a combination of the above (see, for example. Wanisch et al. (2009) Molecular Therapy 17: 1316-1332).

The lentiviral vector of the present invention may be replication-defective and integrating. The lentiviral vector of the present invention may be replication-defective, integrating, and self-inactivating. The lentiviral vector of the present invention may be replication-defective, integrating, self-inactivating, and HIV-derived.

The lentiviral vector of the present invention may be a lentiviral particle. A “lentiviral particle” may refer to an enveloped lentiviral genome. Lentiviral particles may be generated by co-transfection of a plasmid containing a lentiviral genome (e.g. a “transfer vector”) with helper plasmids (e.g. “packaging vectors” encoding gag-pol and/or rev, and “envelope vectors” encoding env) into host cells and harvesting of the lentivirus-containing supernatant afterwards.

The lentiviral vector of the present invention may be pseudotyped. Pseudotyping lentiviral vectors with naturally occurring or engineered lentiviral envelopes can allow targeted transduction of specific cell types (see, for example, Joglekar et al. (2017) Human Gene Therapy Methods 28: 291-301). Suitably, the lentiviral vector is pseudotyped to allow transduction of liver cells (e.g. hepatocytes).

The lentiviral vector of the present invention may be VSV-G pseudotyped. Vesicular stomatitis virus G protein (VSV-G) is a commonly used envelope protein for pseudotyping. VSV-G is a trimeric protein that binds phosphatidylserine and low-density lipoprotein receptors on a cell surface to endocytose into the cell.

The lentiviral vector of the present invention may be replication-defective, integrating, and VSV-G pseudotyped. The lentiviral vector of the present invention may be replication-defective, integrating, self-inactivating, and VSV-G pseudotyped. The lentiviral vector of the present invention may be replication-defective, integrating self-inactivating, HIV-derived, and VSV-G pseudotyped.

In some embodiments, the lentiviral vector of the present invention: (i) comprises one or more miRNA target sequence (e.g. which suppresses LDLR expression in antigen-presenting cells); (ii) is a CD47^{high} lentiviral vector; and/or (iii) is a MHC-I^{free} lentiviral vector. In preferred embodiments, the lentiviral vector of the present invention comprises one or more miRNA target sequence (e.g. which suppresses LDLR expression in antigen-presenting cells) and is a CD47^{high}/MHC-I^{free} lentiviral vector. Each of these features may reduce immune responses following administration.

miRNA Target Sequence

The lentiviral vector of the present invention may comprise one or more miRNA target sequence. The one or more miRNA target sequence may be operably linked to the LDLR-coding sequence. The term “operably linked” may mean that the components described are in a relationship permitting them to function in their intended manner.

MicroRNA (miRNA) genes are scattered across all human chromosomes, except for the Y chromosome. Similar to protein-coding genes, miRNAs are usually transcribed from polymerase-II promoters, generating a so-called primary miRNA transcript (pri-miRNA). From the pri-miRNA, a stem loop of about 60 nucleotides in length, called miRNA precursor (pre-miRNA), is excised leaving a 5′ phosphate and a 2 bp long, 3′ overhang. The pre-miRNA is then actively transported from the nucleus to the cytoplasm. Then, Dicer performs a double strand cut at the other end of the stem loop, generating a 19-24 bp duplex, which is composed of the mature miRNA and the opposite strand of the duplex, called miRNA*. One strand of the duplex is selectively loaded into the RNA-induced silencing complex (RISC), and accumulates as the mature microRNA. This strand is usually the one whose 5′ end is less tightly paired to its complement. However, there are some miRNAs that support accumulation of both duplex strands to similar extent.

Once loaded into RISC, the guide strand of the mature microRNA interacts with mRNA target sequences preferentially found in the 3′ untranslated region (3′UTR) of protein-coding genes. If the whole guide strand sequence is perfectly complementary to the mRNA target, the mRNA is endonucleolytically cleaved. If only the seed sequence (i.e. nucleotides 2-8 counted from the 5′ end of the miRNA) is perfectly complementary to the target mRNA, RNAi may act through alternative mechanisms leading to translational repression.

Expression of the protein from the protein-coding sequence (i.e. “transgene expression”) may be regulated by one or more endogenous miRNAs using one or more corresponding miRNA target sequence. Using this method, one or more miRNAs endogenously expressed in a cell prevent or reduce transgene (e.g. LDLR) expression in that cell by interacting with its corresponding miRNA target sequence positioned in the lentiviral genome.

Suitable miRNA target sequences which suppress transgene expression in specific cells will be known to the skilled person. Determining a miRNA with the desired expression profile may be achieved using techniques known to those skilled in the art. For example, a mammalian microRNA expression atlas is described in Landgraf et al. (2007) Cell 129: 1401-1414 and the distribution of miRNA expression across human tissues is described in Ludwig et al. (2016) Nucleic Acids Research 44: 3865-3877. Once a miRNA has been identified, the corresponding target sequence can readily be determined using, for example, a microRNA database, such as miRBase (Griffiths-Jones et al. (2007) Nucleic Acids Research 36 (suppl_1): D154-D158).

A miRNA target sequence may be fully or partially complementary to the corresponding miRNA. The term “fully complementary”, as used herein, may mean that the target sequence has a nucleic acid sequence which is 100% complementary to the sequence of the miRNA which recognises it. The term “partially complementary”, as used herein, may mean that the target sequence is only in part complementary to the sequence of the miRNA which recognises it, whereby the partially complementary sequence is still recognised by the miRNA. In other words, a partially complementary target sequence in the context of the present invention is effective in recognising the corresponding miRNA and effecting prevention or reduction of transgene expression in cells expressing that miRNA. Suitably, a partially complementary miRNA target sequence may be fully complementary to the miRNA seed sequence.

Including more than one copy of a miRNA target sequence in a lentiviral vector may increase the effectiveness of the system. Also, different miRNA target sequences can be included. For example, the protein-coding (e.g. LDLR-coding) sequence may be operably linked to more than one miRNA target sequence, which may or may not be different. The miRNA target sequences may be in tandem, but other arrangements are envisaged. The lentiviral vector may, for example, comprise 1, 2, 3, 4, 5, 6, 7 or 8 copies of the same or different miRNA target sequences. Suitably, the lentiviral vector comprises 4 miRNA target sequences of each miRNA target sequence.

Copies of miRNA target sequences may be separated by a spacer sequence. A spacer sequence may comprise, for example, at least one, at least two, at least three, at least four or at least five nucleotide bases.

Suitably, the lentiviral vector comprises one or more miRNA target sequence, two or more miRNA target sequences, three or more miRNA target sequences, or four or more miRNA target sequences. Suitably, the protein-coding (e.g. LDLR-coding) sequence is operably linked to one or more miRNA target sequence, two or more miRNA target sequences, three or more miRNA target sequences, or four or more miRNA target sequences. In some embodiments, the protein-coding sequence is operably linked to four miRNA target sequences.

The miRNA target sequence may be a human miRNA target sequence. Suitably, the miRNA target sequence is a −5p or −3p miRNA target sequence.

The one or more miRNA target sequence may suppress transgene expression in one or more cells other than liver cells (e.g. hepatocytes).

The one or more miRNA target sequence may suppress transgene expression in hematopoietic-lineage cells. Hematopoietic stem cells give rise to different types of blood cells, in lines called myeloid and lymphoid. As used herein, “hematopoietic-lineage cells” may include myeloid cells and lymphoid cells. Myeloid cells may include monocytes, macrophages, neutrophils, basophils and eosinophils. Lymphoid cells may include T cells, B cells, natural killer cells and innate lymphoid cells.

The one or more miRNA target sequence may suppress transgene expression in antigen-presenting cells. As used herein, an “antigen presenting cell” (APC) may refer to a cell that displays antigen bound by major histocompatibility complex (MHC) proteins on its surface. APCs may be hematopoietic-lineage cells. The antigen-presenting cells may be professional antigen-presenting cells. Professional APCs specialise in presenting antigens to T cells and may include macrophages, B cells and dendritic cells. Suitably, the APCs are splenic and/or hepatic APCs.

The one or more miRNA target sequence may suppress transgene expression in hematopoietic-lineage antigen-presenting cells.

By preventing transgene expression in antigen-presenting cells, while permitting high levels of expression in other cells, miRNA regulation may enable strong and stable gene transfer in the absence of an immune response.

As used herein, the term “suppress expression” may refer to a reduction of expression in the relevant cell type(s) of a transgene to which the one or more miRNA target sequence is operably linked as compared to transgene expression in the absence of the one or more miRNA target sequence, but under otherwise substantially identical conditions. In some embodiments, transgene expression is suppressed by at least 50%. In some embodiments, transgene expression is suppressed by at least 60%, at least 70%, at least 80%, at least 90%, or at least 95%. In some embodiments, transgene expression is substantially prevented, for example is not detectable.

The miRNA-mediated approach for restricting gene expression has several advantages over other strategies of regulating transgenes. Although using tissue-specific promoters can successfully limit expression to target cells, leaky expression in a fraction of non-target cells is observed. This occurs because the reconstituted promoter, modified for inclusion into a vector system, often loses some of its cell specificity and also because vector integration near active promoters and enhancers can activate the tissue-specific promoter and drive transgene expression. In contrast, because miRNA-mediated silencing occurs at the post-transcriptional level, promoter and enhancer trapping is irrelevant. As such, miRNA-regulation can be used to effectively de-target transgene expression from a particular cell type, while still allowing for broad tissue expression. miRNA regulation may also be used as in combination with tissue-specific promoter/enhancers. By including the miRNA target sequence in expression cassettes already under the control of a tissue-specific promoter, an additional layer of regulation is added which may eliminate off-target expression.

Exemplary miRNA target sequences which suppress transgene expression in hematopoietic-lineage cells and/or antigen-presenting cells, include, but are not limited to, miR-142, miR-181, miR-223 and miR-155 target sequences. Other miRNA target sequences which suppress transgene expression in hematopoietic-lineage cells and/or antigen-presenting cells are known in the art (see, for example, Ghafouri-Fard et al. (2021) Non-coding RNA research 6: 8-14). miRNAs which are expressed in hematopoietic-lineage cells and/or antigen-presenting cells interact with the corresponding miRNA target sequence and reduce the expression of the target gene.

Further miRNA target sequences that suppress transgene expression in hematopoietic-lineage cells and/or antigen-presenting cells can be identified by any suitable method, for example miRNA expression analysis as described in Monticelli et al. (2005) Genome Biology 6: 1-15.

Suitably, the one or more miRNA target sequence comprises or consists of: (i) one or more (e.g. 1, 2, 3, 4, 5, 6, 7, 8) miR-142 target sequence; (ii) one or more (e.g. 1, 2, 3, 4, 5, 6, 7, 8) miR-181 target sequence; (iii) one or more (e.g. 1, 2, 3, 4, 5, 6, 7, 8) miR-223 target sequence; and/or (iv) one or more (e.g. 1, 2, 3, 4, 5, 6, 7, 8) miR-155 target sequence.

In some embodiments, the one or more miRNA target sequence comprises or consists of: (i) two or more miR-142 target sequences; (ii) two or more miR-181 target sequences; (iii) two or more miR-223 target sequences; and/or (iv) two or more miR-155 target sequences. In some embodiments, the one or more miRNA target sequence comprises or consists of: (i) at least four miR-142 target sequences; (ii) at least four miR-181 target sequences; (iii) at least four miR-223 target sequences; and/or (iv) at least four miR-155 target sequences. In some embodiments, the one or more miRNA target sequence comprises or consists of: (i) four miR-142 target sequences; (ii) four miR-181 target sequences; (iii) four miR-223 target sequences; and/or (iv) four miR-155 target sequences. Suitably, the target sequences are separated by spacer sequences.

In some embodiments, the one or more miRNA target sequence comprises or consists of one or more (e.g. 1, 2, 3, 4, 5, 6, 7, 8) miR-142 target sequence. In some embodiments, the one or more miRNA target sequence comprises or consists of two or more miR-142 target sequences. In some embodiments, the one or more miRNA target sequence comprises or consists of three or more miR-142 target sequences. In some embodiments, the one or more miRNA target sequence comprises or consists of four or more miR-142 target sequences. In some embodiments, the one or more miRNA target sequence comprises or consists of four miR-142 target sequences. Suitably, the target sequences are separated by spacer sequences.

The miR-142 target sequence may be a human miRNA target sequence. Suitably, the miR-142 target sequence is a miR-142-5p or miR-142-3p miRNA target sequence. In some embodiments, the miR-142 target sequence is a miR-142-3p miRNA target sequence.

In some embodiments, the miR-142 target sequence comprises or consists of a nucleotide sequence that has at least 80% sequence identity to SEQ ID NO: 6 or a fragment thereof. Suitably, the miR-142 target sequence comprises or consists of a nucleotide sequence that has at least 85%, at least 90% or at least 95% sequence to SEQ ID NO: 6 or a fragment thereof.

In some embodiments, the miR-142 target sequence comprises or consists of the nucleotide sequence SEQ ID NO: 6 or a fragment thereof.

Example miR-142 target sequence:

(SEQ ID NO: 6)
TCCATAAAGTAGGAAACACTACA

In some embodiments, the one or more miRNA target sequence comprises or consists of a nucleotide sequence that has at least 70% sequence identity to SEQ ID NO: 7 or a fragment thereof.

In some embodiments, the one or more miRNA target sequence comprises or consists of a nucleotide sequence that has at least 75%, at least 80%, at least 85%, at least 90%, at least 95%, at least 96%, at least 97%, at least 98%, at least 99% or 100% sequence identity to SEQ ID NO: 7 or a fragment thereof.

In some embodiments, the one or more miRNA target sequence comprises or consists of the nucleotide sequence of SEQ ID NO: 7 or a fragment thereof.

(SEQ ID NO: 7)
TCCATAAAGTAGGAAACACTACACGATTCCATAAAGTAGGAAACACTAC
AACCGGTTCCATAAAGTAGGAAACACTACATCACTCCATAAAGTAGGAA
ACACTACA

### CD47^{high} Lentiviral Vectors

The lentiviral vector of the present invention may be a CD47^{high} lentiviral vector. As used herein, a “CD47^{high} lentiviral vector” may refer to a lentiviral vector with increased levels of CD47 (or a fragment thereof) on its surface. A CD47^{high} lentiviral vector may have reduced uptake by professional phagocytes. In some embodiments the surface of a CD47^{high} lentiviral vector comprises a higher level of CD47 protein than a control lentiviral vector produced in HEK293T cells (AT CC® CRL-1 1268™).

CD47 (Cluster of Differentiation 47) also known as integrin associated protein (IAP) is a transmembrane protein that in humans is encoded by the CD47 gene. Phagocytosis is physiologically inhibited by CD47, which is a ubiquitously expressed ligand of signal regulatory protein a (SIRP-α) receptor, that is expressed by professional phagocytes. CD47 may be incorporated into lentiviral vectors when they bud from producer cells.

The lentiviral vector of the present invention may comprise one or more CD47 polypeptides (or a fragment thereof) on its surface. The amount of CD47 (or a fragment thereof) on the surface may be enough to reduce uptake by professional phagocytes. Any suitable assay to quantify the amount of CD47 polypeptides (or fragments thereof) present on the surface of the lentiviral vector may be used.

In some embodiments, the density of CD47 polypeptides (or fragments thereof) may be determined by immunostaining for CD47 and total internal reflection fluorescence microscopy, for example as described in US20100316570A1. The CD47 polypeptides (or fragments thereof) may be present in a density of at least about 20 molecules/μm², at least about 25 molecules/μm², at least about 30 molecules/μm², at least about 35 molecules/μm², at least about 40 molecules/μm², at least about 45 molecules/μm², at least about 50 molecules/μm², at least about 60 molecules/μm², at least about 70 molecules/μm², at least about 80 molecules/μm², at least about 90 molecules/μm², at least about 100 molecules/μm², at least about 150 molecules/μm², at least about 200 molecules/μm², at least about 250 molecules/μm², at least about 300 molecules/μm², at least about 350 molecules/μm², at least about 400 molecules/μm², at least about 450 molecules/μm², at least about 500 molecules/μm², at least about 600 molecules/μm², at least about 700 molecules/μm², at least about 800 molecules/μm², at least about 900 molecules/μm² or at least about 1000 molecules/μm². The CD47 polypeptides (or fragments thereof) may be present in a density of about 1000 molecules/μm² or less, about 500 molecules/μm² or less or about 250 molecules/μm² or less. The CD47 polypeptides (or fragments thereof) may be present in a density of from about 20 molecules/μm² to about 1000 molecules/μm², from about 20 molecules/μm² to about 500 molecules/μm² or from about 20 molecules/μm² to about 250 molecules/μm².

In some embodiments, the amount of CD47 polypeptides (or fragments thereof) may be determined by immunostaining for CD47 and electron microscopy, as described in Milani et al. (2019) Science Translational Medicine 11: eaav7325. The CD47 polypeptides (or fragments thereof) may be detected in an amount of at least about 10 gold particles/lentiviral particle, at least about 15 gold particles/lentiviral particle or at least about 20 gold particles/lentiviral particle. The CD47 polypeptides (or fragments thereof) may be detected in an amount of about 100 gold particles/lentiviral particle or less, about 80 gold particles/lentiviral particle or less or about 60 gold particles/lentiviral particle or less. The CD47 polypeptides (or fragments thereof) may be detected in an amount of from about 10 to about 100 gold particles/lentiviral particle, from about 15 to about 80 gold particles/lentiviral particle or from about 20 to about 60 gold particles/lentiviral particle.

The lentiviral vector of the present invention may be obtained from a CD47^{high} producer cell. As used herein, a “CD47^{high} producer cell” may refer to a producer cell with increased levels of CD47 (or a fragment thereof) on its surface.

A CD47^{high} producer cell may be genetically engineered to increase expression of CD47 (or a fragment thereof) on the cell surface. For example, the producer cell may comprise a vector encoding CD47 (or a fragment thereof) or may be edited to introduce a nucleotide sequence encoding CD47 (or a fragment thereof) into its genome. Suitably, the producer cell is transduced with a viral vector encoding a CD47 polypeptide (or a fragment thereof).

A CD47^{high} producer cell may have a higher concentration of CD47 (or a fragment thereof) on its surface than an unmodified producer cell. Suitably, the producer cell has at least about 2-fold, at least about 3-fold, at least about 4-fold, at least about 5-fold, at least about 10-fold, at least about 20-fold or at least about 30-fold more CD47 on its cell surface than an unmodified producer cell. Suitably, the producer cell has from about 5-fold to about 30-fold more CD47 (or a fragment thereof) on its cell surface than an unmodified producer cell.

Suitably, the lentiviral vector of the present has a higher concentration of CD47 (or a fragment thereof) on its surface than a lentiviral vector obtained from an unmodified producer cell. Suitably, the lentiviral vector has at least about 2-fold, at least about 3-fold, at least about 4-fold, at least about 5-fold, at least about 10-fold, at least about 20-fold, at least about 30-fold, at least about 40-fold or at least about 50-fold more CD47 (or a fragment thereof) on its surface than a lentiviral vector obtained from an unmodified producer cell. Suitably, the lentiviral vector has from about 5-fold to about 30-fold more CD47 (or a fragment thereof) on its surface than a lentiviral vector obtained from an unmodified producer cell.

CD47 is a member of the immunoglobulin (Ig) superfamily of membrane proteins, with a single IgV-like domain at its N-terminus, a highly hydrophobic stretch with five membrane-spanning segments and an alternatively spliced cytoplasmic C-terminus ranging in length from 3 to 36 amino acids. Mouse, rat, bovine and human CD47 molecules have been cloned and show about 70% overall amino acid identity (see, for example, Brown et al. (2001) Trends Cell Biology 11: 130-135).

The CD47 polypeptide (or a fragment thereof) may be a human CD47 polypeptide (or a fragment thereof). A CD47 polypeptide may have an amino acid sequence of UniProtKB Q08722.

Exemplary CD47 polypeptides are provided by SEQ ID NOs: 8-11. Suitably, a CD47 polypeptide comprises an amino acid sequence that has at least about 70%, at least about 75%, at least about 80%, at least about 85%, at least about 90%, at least about 95%, at least about 96%, at least about 97%, at least about 98%, at least about 99% or about 100% sequence identity to any of SEQ ID NOs: 8-11. Suitably, a CD47 polypeptide comprises or consists of the amino acid sequence of any of SEQ ID NOs: 8-11.

Example CD47 amino acid sequences:

(SEQ ID NO: 8)
MWPLVAALLLGSACCGSAQLLFNKTKSVEFTFCNDTVVIPCFVTNMEAQNTTEVYVKWKFKG
RDIYTFDGALNKSTVPTDFSSAKIEVSQLLKGDASLKMDKSDAVSHTGNYTCEVTELTREGE
TIIELKYRVVSWFSPNENILIVIFPIFAILLFWGQFGIKTLKYRSGGMDEKTIALLVAGLVI
TVIVIVGAILFVPGEYSLKNATGLGLIVTSTGILILLHYYVFSTAIGLTSFVIAILVIQVIA
YILAVVGLSLCIAACIPMHGPLLISGLSILALAQLLGLVYMKFVASNQKTIQPPRNN
(SEQ ID NO: 9)
MWPLVAALLLGSACCGSAQLLFNKTKSVEFTFCNDTVVIPCFVTNMEAQNTTEVYVKWKFKG
RDIYTFDGALNKSTVPTDFSSAKIEVSQLLKGDASLKMDKSDAVSHTGNYTCEVTELTREGE
TIIELKYRVVSWFSPNENILIVIFPIFAILLFWGQFGIKTLKYRSGGMDEKTIALLVAGLVI
TVIVIVGAILFVPGEYSLKNATGLGLIVTSTGILILLHYYVFSTAIGLTSFVIAILVIQVIA
YILAVVGLSLCIAACIPMHGPLLISGLSILALAQLLGLVYMKFVASNQKTIQPPRKAVEEPL
NAFKESKGMMNDE
(SEQ ID NO: 10)
MWPLVAALLLGSACCGSAQLLFNKTKSVEFTFCNDTVVIPCFVTNMEAQNTTEVYVKWKFKG
RDIYTFDGALNKSTVPTDFSSAKIEVSQLLKGDASLKMDKSDAVSHTGNYTCEVTELTREGE
TIIELKYRVVSWFSPNENILIVIFPIFAILLFWGQFGIKTLKYRSGGMDEKTIALLVAGLVI
TVIVIVGAILFVPGEYSLKNATGLGLIVTSTGILILLHYYVFSTAIGLTSFVIAILVIQVIA
YILAVVGLSLCIAACIPMHGPLLISGLSILALAQLLGLVYMKFV
(SEQ ID NO: 11)
MWPLVAALLLGSACCGSAQLLFNKTKSVEFTFCNDTVVIPCFVTNMEAQNTTEVYVKWKFKG
RDIYTFDGALNKSTVPTDFSSAKIEVSQLLKGDASLKMDKSDAVSHTGNYTCEVTELTREGE
TIIELKYRVVSWFSPNENILIVIFPIFAILLFWGQFGIKTLKYRSGGMDEKTIALLVAGLVI
TVIVIVGAILFVPGEYSLKNATGLGLIVTSTGILILLHYYVFSTAIGLTSFVIAILVIQVIA
YILAVVGLSLCIAACIPMHGPLLISGLSILALAQLLGLVYMKFVASNQKTIQPPRKAVEEPL
N

Exemplary CD47 polypeptides excluding the signal peptide are provided by SEQ ID NOs: 12-15. Suitably, a CD47 polypeptide comprises an amino acid sequence that has at least about 70%, at least about 75%, at least about 80%, at least about 85%, at least about 90%, at least about 95%, at least about 96%, at least about 97%, at least about 98%, at least about 99% or about 100% sequence identity to any of SEQ ID NOs: 12-15. Suitably, a CD47 polypeptide comprises or consists of the amino acid sequence of any of SEQ ID NOs: 12-15.

Example CD47 amino acid sequences excluding signal peptide:

(SEQ ID NO: 12)
QLLFNKTKSVEFTFCNDTVVIPCFVTNMEAQNTTEVYVKWKFKGRDIYTFDGALNKSTVPTD
FSSAKIEVSQLLKGDASLKMDKSDAVSHTGNYTCEVTELTREGETIIELKYRVVSWFSPNEN
ILIVIFPIFAILLFWGQFGIKTLKYRSGGMDEKTIALLVAGLVITVIVIVGAILFVPGEYSL
KNATGLGLIVTSTGILILLHYYVFSTAIGLTSFVIAILVIQVIAYILAVVGLSLCIAACIPM
HGPLLISGLSILALAQLLGLVYMKFVASNQKTIQPPRNN
(SEQ ID NO: 13)
QLLFNKTKSVEFTFCNDTVVIPCFVTNMEAQNTTEVYVKWKFKGRDIYTFDGALNKSTVPTD
FSSAKIEVSQLLKGDASLKMDKSDAVSHTGNYTCEVTELTREGETIIELKYRVVSWFSPNEN
ILIVIFPIFAILLFWGQFGIKTLKYRSGGMDEKTIALLVAGLVITVIVIVGAILFVPGEYSL
KNATGLGLIVTSTGILILLHYYVFSTAIGLTSFVIAILVIQVIAYILAVVGLSLCIAACIPM
HGPLLISGLSILALAQLLGLVYMKFVASNQKTIQPPRKAVEEPLNAFKESKGMMNDE
(SEQ ID NO: 14)
QLLFNKTKSVEFTFCNDTVVIPCFVTNMEAQNTTEVYVKWKFKGRDIYTFDGALNKSTVPTD
FSSAKIEVSQLLKGDASLKMDKSDAVSHTGNYTCEVTELTREGETIIELKYRVVSWFSPNEN
ILIVIFPIFAILLFWGQFGIKTLKYRSGGMDEKTIALLVAGLVITVIVIVGAILFVPGEYSL
KNATGLGLIVTSTGILILLHYYVFSTAIGLTSFVIAILVIQVIAYILAVVGLSLCIAACIPM
HGPLLISGLSILALAQLLGLVYMKFV
(SEQ ID NO: 15)
QLLFNKTKSVEFTFCNDTVVIPCFVTNMEAQNTTEVYVKWKFKGRDIYTFDGALNKSTVPTD
FSSAKIEVSQLLKGDASLKMDKSDAVSHTGNYTCEVTELTREGETIIELKYRVVSWFSPNEN
ILIVIFPIFAILLFWGQFGIKTLKYRSGGMDEKTIALLVAGLVITVIVIVGAILFVPGEYSL
KNATGLGLIVTSTGILILLHYYVFSTAIGLTSFVIAILVIQVIAYILAVVGLSLCIAACIPM
HGPLLISGLSILALAQLLGLVYMKFVASNQKTIQPPRKAVEEPLN

The skilled person would be able to generate variants and/or fragments, for example based on conservative substitutions and/or the known structural and functional features of CD47. These are described, for instance in Fenalti et al. (2021) Nature Communications 12: 1-14.

Suitably, a fragment of CD47 and/or CD47 variant retains the ability to inhibit phagocytosis. Suitably, a CD47 fragment and/or CD47 variant may comprise the extracellular domain of CD47. The extracellular domain of human CD47 may interact with SIRP-α and inhibit phagocytosis. Optionally, a CD47 fragment and/or CD47 variant comprises the transmembrane domain of CD47. The domains may be linked by inter-domain linker(s). The fragment and/or variant may have at least 40%, at least 50%, at least 60%, at least 70%, at least 80%, at least 90%, at least 95% or 100% of the activity of a full-length CD47 polypeptide.

Suitably, a variant of SEQ ID NO: 8 may comprise one or more variation selected from V5I, C14W, C15R, F22L, S27F, F30L, F32Y, T36S, V38L, V38I, F42V, T44A, N50S, T51A, T52S, T52A, V561, R63K, A71T, S75Y, T76A, P78L, P78S, P78A, S82R, S82N, S83T, K85N, K85E, V88A, V88L, V881, Q90R, L91F, K93N, M1001, M100V, D101G, K102R, K102T, S107L, I126F, 1127V, K130Q, R132H, S138F, V146I, I150V, I153V, S169A, G170R, G170S, G171S, D173Y, I177V, A178G, V181I, V185A, I186V, V188A, I189T, I191V, V198I, A207S, T215I, I219M, Y226C, A231S, T235A, S236F, A240V, A240T, I241V, V243I, I244T, V246L, Y249F, A252S, A252T, V254A, S257T, 1264M, 1264L, M2661, M266T, M266V, V287I, V292A, N295S, N295D, Q296L, P302S, N304S and N304D. These are considered to be tolerated, benign, and/or likely benign variations as predicted by SIFT, PolyPhen, CADD, REVEL and MetaLR.

Suitably, a variant of SEQ ID NO: 9 may comprise one or more variation selected from P3L, A6P, F22L, S27F, F30L, F32Y, T36S, V38L, V38I, F42V, N50S, T51A, T52S, T52A, V561, R63K, A71T, S75Y, T76A, P78L, P78S, P78A, S82R, S82N, S83T, K85N, K85E, V88A, V88L, V881, Q90R, L91F, K93N, M1001, M100V, D101G, K102R, K102T, S107L, I126F, 1127V, K130Q, R132H, S138F, V146, I150V, I153V, S169A, G170R, G170S, G171S, I177V, A178G, V181I, I186V, V188A, I189T, I191V, V198I, A207S, T215I, I219M, Y226C, A231S, T235A, A240V, A240T, I241V, V243I, I244T, V246L, Y249F, A252S, A252T, V254A, 1264M, 1264L, M2661, M266T, M266V, V287I, V292A, N295S, N295D and Q296L. These are considered to be tolerated, benign, and/or likely benign variations as predicted by SIFT, PolyPhen, CADD, REVEL, and MetaLR.

An exemplary CD47 fragment is provided by SEQ ID NO: 16. Suitably, a CD47 fragment comprises an amino acid sequence that has at least about 70%, at least about 75%, at least about 80%, at least about 85%, at least about 90%, at least about 95%, at least about 96%, at least about 97%, at least about 98%, at least about 99% or about 100% sequence identity to SEQ ID NO: 16. Suitably, a CD47 fragment comprises or consists of the amino acid sequence of SEQ ID NO: 16.

Example CD47 fragment:

(SEQ ID NO: 16)
MWPLVAALLLGSACCGSAQLLFNKTKSVEFTFCNDTVVIPCFVTNMEAQ
NTTEVYVKWKFKGRDIYTFDGALNKSTVPTDFSSAKIEVSQLLKGDASL
KMDKSDAVSHTGNYTCEVTELTREGETIIELKYRVVSWFSPN

An exemplary CD47 fragment excluding the signal peptide is provided by SEQ ID NO: 17. Suitably, a CD47 fragment comprises an amino acid sequence that has at least about 70%, at least about 75%, at least about 80%, at least about 85%, at least about 90%, at least about 95%, at least about 96%, at least about 97%, at least about 98%, at least about 99% or about 100% identity to SEQ ID NO: 17. Suitably, a CD47 fragment comprises or consists of the amino acid sequence of SEQ ID NO: 17.

Example CD47 fragment excluding signal peptide:

(SEQ ID NO: 17)
QLLFNKTKSVEFTFCNDTVVIPCFVTNMEAQNTTEVYVKWKFKGRDIYT
FDGALNKSTVPTDFSSAKIEVSQLLKGDASLKMDKSDAVSHTGNYTCEV
TELTREGETIIELKYRVVSWFSPN

MHC-I^{low} or MHC-I^{free} Lentiviral Vectors

The lentiviral vector of the present invention may be a MHC-I^{low} lentiviral vector or a MHC-I^{free} lentiviral vector. In preferred embodiments, the lentiviral vector of the present invention is a MHC-I^{free} lentiviral vector.

As used herein, a “MHC-I^{low} lentiviral vector” may refer to a lentiviral vector with reduced levels of one or more MHC-I molecules on its surface (i.e. reduced levels of surface-exposed MHC-I molecules). The number of surface-exposed MHC-I molecules may be reduced such that the immune response to the MHC-I is decreased to a therapeutically relevant degree.

As used herein, a “MHC-I^{free} lentiviral vector” may refer to a lentiviral vector which is substantially devoid of (or free of) one or more MHC-I molecules on its surface (i.e. substantially devoid of (or free of) surface-exposed MHC-I molecules). Specifically, the surface of the lentiviral vector may not comprise MHC-I.

The major histocompatibility complex class I (MHC-1) is a heterodimeric membrane protein that is displayed on the outer leaflet of the cell membrane (see, for example, Penn et al. (2005) Major histocompatibility complex (MHC). eLS). MHC-I functions to bind and display peptide fragments of proteins to the extracellular environment where they may be recognised by CD8+ cytotoxic T cells. Peptide fragments generated from normal cellular proteins will not activate cytotoxic T cells due to central and peripheral tolerance mechanisms. However, foreign peptides (e.g. those originating from viral proteins) will cause activation of an immune response to destroy the cell. An allogeneic MHC-I protein itself may be recognised by the immune system. For example, antibodies may bind MHC-I epitopes directly. As a result, lentiviral vectors that comprise MHC-I molecules originating from an allogeneic source may be targeted and neutralised by the immune system.

The term “MHC-1 molecules” may refer to human MHC-I molecules. Human MHC-I is also referred to as human leukocyte antigen class I (HLA-I) and is expressed on almost all nucleated cells. HLA-I consists of two polypeptide chains, an HLA-I heavy chain (α chain) and β2 microglobulin (β2M or β chain). The HLA-I α chain and β2M are linked non-covalently. The HLA-I α chain is polymorphic. Six HLA-I α chains have been identified to date, including three classical, highly polymorphic a chains (HLA-A, HLA-B and HLA-C) and three non-classical, less polymorphic (HLA-E, HLA-F and HLA-G) a chains. The MHC-I molecules may comprise or consist of HLA-A, HLA-B and HLA-C molecules, which comprise an invariant β2M sequence.

The term “MHC-1 molecules” may also include variant MHC-I sequences, such as polymorphisms of HLA-I α chain sequences and/or β2M sequences. For example, variant MHC-I sequences may include HLA-I α chain sequences and/or β2M sequences with single nucleotide polymorphisms (SNPs) or multiple SNPs.

Any suitable assay to quantify the amount of MHC-I molecules present on the surface of the lentiviral vector may be used.

In some embodiments, the amount of MHC-I molecules may be determined by immunostaining for MHC-I and electron microscopy, for example as described in Milani et al. (2017) EMBO Molecular Medicine 9: 1558-1573. The MHC-I molecules may be detected in an amount of less than about 10 gold particles/lentiviral particle, less than about 9 gold particles/lentiviral particle, less than about 8 gold particles/lentiviral particle, less than about 7 gold particles/lentiviral particle, less than about 6 gold particles/lentiviral particle, less than about 5 gold particles/lentiviral particle, less than about 4 gold particles/lentiviral particle, less than about 3 gold particles/lentiviral particle, less than about 2 gold particles/lentiviral particle, less than about 1 gold particle/lentiviral particle or about 0 gold particles/lentiviral particle. The MHC-I molecules may be undetectable (e.g. the amount of gold particles detected may not be significantly higher than background levels).

The lentiviral vector of the present invention may be obtained from a MHC-I^{low} producer cell or a MHC-I^{free} producer cell. In preferred embodiments, the lentiviral vector of the present invention is obtained from a MHC-I^{free} producer cell. As used herein, a “MHC-I^{low} producer cell” may refer to a producer cell with reduced levels of one or more MHC-I molecule on its surface. As used herein, a “MHC-I^{free} producer cell” may refer to a producer cell which is substantially devoid of or free of one or more MHC-I molecule on its surface. Specifically, the surface of the lentiviral vector may not comprise MHC-I.

A MHC-I^{low} or MHC-I^{free} producer cell may be genetically engineered to decrease expression of MHC-I on the cell surface. For example, the cell may comprise a genetically engineered disruption of a gene encoding β2-microglobulin and/or a genetically engineered disruption of a gene encoding an MHC-I α chain.

Methods for genetic engineering to decrease protein expression are known in the art. For example, this may be achieved by targeted gene knockout. To decrease protein expression, the gene encoding the protein itself or its regulatory sequence (e.g. its promoter) may be knocked out. Knockout may be achieved by deletion of a section of the coding nucleic acid sequence, which may delete a section of the protein essential for expression or stability, or alter the reading frame of the coding sequence or by base-editing. Suitable methods for targeted gene knockout include use of zinc finger nucleases (ZFNs), transcription activator-like effector nucleases (TALENs) and CRISPR/Cas-based RNA-guided nucleases (see e.g. Gaj et al. (2013) Trends Biotechnol 31: 397-405). For example, the CRISPR/Cas9 RNA-guided nuclease may be used to catalyse a double strand break at a specific locus in the genome if provided with appropriate RNA guides designed to bind that locus. Cas9 and the guide RNA may be delivered to a target cell by transfection of vectors encoding the protein and RNA. Cells attempt to repair any double strand breaks in their DNA using the non-homologous end joining (NHEJ) pathway. This is an error-prone mechanism which inserts random nucleotides and often disrupts the reading frame of the targeted gene. Alternatively, the genetic engineering to decrease protein expression may be accomplished using RNAi techniques, microRNA or antisense RNA to suppress expression of the target gene.

Once the targeted gene knockout or suppression of expression approach has been carried out, the resulting population of cells may be screened to select and enrich for those cells exhibiting the phenotype of interest, for example decreased expression of surface-exposed MHC-I. Suitable techniques for screening and enrichment are known in the art and include flow cytometry and fluorescence-activated cell sorting (FACS).

In some embodiments, the producer cell comprises a genetically engineered disruption of a gene encoding β2-microglobulin. P2-microglobulin stabilises MHC-I, thus cells deficient in P2-microglobulin will exhibit decreased expression of MHC-I on the surface of the cell. The cell may comprise genetically engineered disruptions in all copies of the gene encoding β2-microglobulin.

In another embodiment, the cell comprises a genetically engineered disruption of one or more gene encoding an MHC-I α chain. The cell may comprise genetically engineered disruptions in all copies of the gene encoding an MHC-I α chain.

The cell may comprise both genetically engineered disruptions of genes encoding β2-microglobulin and genetically engineered disruptions of genes encoding an MHC-I α chain.

Decreased expression of MHC-I on the surface of the cell may refer to a decrease in the number of MHC-I molecules that are expressed on the surface of the cell that has been genetically engineered, in comparison to the number of MHC-I molecules that are expressed on the surface of a cell lacking the genetic engineering, but under otherwise substantially identical conditions. The expression of MHC-I on the surface of the cell may be decreased such that the number of surface-exposed MHC-I molecules is, for example, less than about 50%, 40%, 30%, 25%, 20%, 15%, 10%, 5%, 4%, 3%, 2% or 1% of the number of surface-exposed MHC-I molecules that are displayed in the absence of the genetic engineering. In some embodiments, the expression of MHC-I on the surface of the cell is decreased such that the number of surface-exposed MHC-I molecules is 0% of the number of surface-exposed MHC-I molecules that are displayed in the absence of the genetic engineering.

The expression of MHC-I on the surface of the cell is preferably decreased such that the cell is substantially devoid of surface-exposed MHC-I molecules. In this context, “substantially devoid” may mean that there is a substantial decrease in the number of MHC-I molecules that are expressed on the surface of the cell that has been genetically engineered, in comparison to the number of MHC-I molecules that are expressed on the surface of a cell lacking the genetic engineering, such that the immune response to MHC-I on lentiviral vectors produced by the cell is decreased to a therapeutically useful degree.

Suitably, the lentiviral vector of the present invention has a lower concentration of MHC-I molecules on its surface than a lentiviral vector obtained from an unmodified producer cell. Suitably, the lentiviral vector has less than about 50%, 40%, 30%, 25%, 20%, 15%, 10%, 5%, 4%, 3%, 2% or 1% of the number of surface-exposed MHC-I molecules that are displayed on a lentiviral vector obtained from an unmodified producer cell. In some embodiments, the lentiviral vector has less than about 20% of the number of surface-exposed MHC-I molecules that are displayed on a lentiviral vector obtained from an unmodified producer cell.

In some embodiments, the lentiviral vector of the present invention is substantially devoid of MHC-I molecules on its surface. In this context, “substantially devoid” may mean that there is no detectable immune response due to the molecules on the surface of the lentiviral vector.

In some embodiments, the lentiviral vector of the present invention is free of MHC-I molecules on its surface. In this context, “free” may mean that there are no detectable molecules (e.g. by immunostaining and electron microscopy) on the surface of the lentiviral vector. As used herein, “not detectable” may refer to levels which are not statistically significantly different compared to background levels.

In some embodiments, the lentiviral vector of the present invention has decreased HLA-A, HLA-B and/or HLA-C molecules on its surface. Suitably, the lentiviral vector has less than about 50%, 40%, 30%, 25%, 20%, 15%, 10%, 5%, 4%, 3%, 2% or 1% of the number of surface-exposed HLA-A molecules that are displayed on a lentiviral vector obtained from an unmodified producer cell. Suitably, the lentiviral vector has less than about 50%, 40%, 30%, 25%, 20%, 15%, 10%, 5%, 4%, 3%, 2% or 1% of the number of surface-exposed HLA-B molecules that are displayed on a lentiviral vector obtained from an unmodified producer cell. Suitably, the lentiviral vector has less than about 50%, 40%, 30%, 25%, 20%, 15%, 10%, 5%, 4%, 3%, 2% or 1% of the number of surface-exposed HLA-C molecules that are displayed on a lentiviral vector obtained from an unmodified producer cell.

In some embodiments, the lentiviral vector of the present invention is substantially devoid of HLA-A, HLA-B and/or HLA-C molecules on its surface. In some embodiments, the lentiviral vector of the present invention is substantially devoid of HLA-A, HLA-B and HLA-C molecules on its surface. In some embodiments, the lentiviral vector of the present invention is free of HLA-A, HLA-B and/or HLA-C molecules on its surface. In some embodiments, the lentiviral vector of the present invention is free of HLA-A, HLA-B and HLA-C molecules on its surface.

As described above, an HLA-I molecule consists of two polypeptide chains, an HLA-I heavy chain (α chain) and β2 microglobulin (β2M or βchain). The HLA-I α chain and β2M are linked non-covalently.

The skilled person would readily be able to determine amino acid and nucleic acid sequences of HLA-I α chains. For example, the HLA-I α chains may be identified in a genome sequence using their location within the major histocompatibility complex region of the chromosome (see, for example, Penn et al. (2005) Major histocompatibility complex (MHC). eLS).

HLA-A alpha chains may have an amino acid sequence of UniProtKB P04439. Exemplary HLA-A alpha chains are provided by SEQ ID NOs: 18 and 19. Suitably, an HLA-A alpha chain comprises an amino acid sequence that has at least about 70%, at least about 75%, at least about 80%, at least about 85%, at least about 90%, at least about 95%, at least about 96%, at least about 97%, at least about 98%, at least about 99% or about 100% sequence identity to SEQ ID NO: 18 or 19. Suitably, a HLA-A alpha chain comprises or consists of the amino acid sequence of SEQ ID NO: 18 or 19.

(SEQ ID NO: 18)
MAVMAPRTLLLLLSGALALTQTWAGSHSMRYFFTSVSRPGRGEPRFIAVGYVDDTQFVRFDS
DAASQRMEPRAPWIEQEGPEYWDQETRNVKAQSQTDRVDLGTLRGYYNQSEAGSHTIQIMYG
CDVGSDGRFLRGYRQDAYDGKDYIALNEDLRSWTAADMAAQITKRKWEAAHEAEQLRAYLDG
TCVEWLRRYLENGKETLQRTDPPKTHMTHHPISDHEATLRCWALGFYPAEITLTWQRDGEDQ
TQDTELVETRPAGDGTFQKWAAVVVPSGEEQRYTCHVQHEGLPKPLTLRWELSSQPTIPIVG
IIAGLVLLGAVITGAVVAAVMWRRKSSDRKGGSYTQAASSDSAQGSDVSLTACKV
(SEQ ID NO: 19)
MAVMAPRTLLLLLSGALALTQTWAGSHSMRYFFTSVSRPGRGEPRFIAVGYVDDTQFVRFDS
DAASQRMEPRAPWIEQEGPEYWDQETRNVKAQSQTDRVDLGTLRGYYNQSEAGSHTIQIMYG
CDVGSDGRFLRGYRQDAYDGKDYIALNEDLRSWTAADMAAQITKRKWEAAHAAEQQRAYLEG
RCVEWLRRYLENGKETLQRTDPPKTHMTHHPISDHEATLRCWALGFYPAEITLTWQRDGEDQ
TQDTELVETRPAGDGTFQKWAAVVVPSGEEQRYTCHVQHEGLPKPLTLRWELSSQPTIPIVG
IIAGLVLLGAVITGAVVAAVMWRRKSSGGEGVKDRKGGSYTQAASSDSAQGSDVSLTACKV

HLA-B alpha chains may have an amino acid sequence of UniProtKB P01889. An exemplary HLA-B alpha chain is provided by SEQ ID NO: 20. Suitably, an HLA-B alpha chain comprises an amino acid sequence that has at least about 70%, at least about 75%, at least about 80%, at least about 85%, at least about 90%, at least about 95%, at least about 96%, at least about 97%, at least about 98%, at least about 99% or about 100% sequence identity to SEQ ID NO: 20. Suitably, a HLA-B alpha chain comprises or consists of the amino acid sequence of SEQ ID NO: 20.

(SEQ ID NO: 20)
MLVMAPRTVLLLLSAALALTETWAGSHSMRYFYTSVSRPGRGEPRFISVGYVDDTQFVRFDS
DAASPREEPRAPWIEQEGPEYWDRNTQIYKAQAQTDRESLRNLRGYYNQSEAGSHTLQSMYG
CDVGPDGRLLRGHDQYAYDGKDYIALNEDLRSWTAADTAAQITQRKWEAAREAEQRRAYLEG
ECVEWLRRYLENGKDKLERADPPKTHVTHHPISDHEATLRCWALGFYPAEITLTWQRDGEDQ
TQDTELVETRPAGDRTFQKWAAVVVPSGEEQRYTCHVQHEGLPKPLTLRWEPSSQSTVPIVG
IVAGLAVLAVVVIGAVVAAVMCRRKSSGGKGGSYSQAACSDSAQGSDVSLTA

HLA-C alpha chains may have an amino acid sequence of UniProtKB P10321. Exemplary HLA-C alpha chains are provided by SEQ ID NOs: 21 and 22. Suitably, an HLA-C alpha chain comprises an amino acid sequence that has at least about 70%, at least about 75%, at least about 80%, at least about 85%, at least about 90%, at least about 95%, at least about 96%, at least about 97%, at least about 98%, at least about 99% or about 100% sequence identity to SEQ ID NO: 21 or 22. Suitably, a HLA-C alpha chain comprises or consists of the amino acid sequence of SEQ ID NO: 21 or 22.

(SEQ ID NO: 21)
MRVMAPRALLLLLSGGLALTETWACSHSMRYFDTAVSRPGRGEPRFISVGYVDDTQFVRFDS
DAASPRGEPRAPWVEQEGPEYWDRETQKYKRQAQADRVSLRNLRGYYNQSEDGSHTLQRMSG
CDLGPDGRLLRGYDQSAYDGKDYIALNEDLRSWTAADTAAQITQRKLEAARAAEQLRAYLEG
TCVEWLRRYLENGKETLQRAEPPKTHVTHHPLSDHEATLRCWALGFYPAEITLTWQRDGEDQ
TQDTELVETRPAGDGTFQKWAAVVVPSGQEQRYTCHMQHEGLQEPLTLSWEPSSQPTIPIMG
IVAGLAVLVVLAVLGAVVTAMMCRRKSSGGKGGSCSQAACSNSAQGSDESLITCKA
(SEQ ID NO: 22)
MRVMAPRALLLLLSGGLALTETWACSHSMRYFDTAVSRPGRGEPRFISVGYVDDTQFVRFDS
DAASPRGEPRAPWVEQEGPEYWDRETQKYKRQAQADRVSLRNLRGYYNQSEDGSHTLQRMSG
CDLGPDGRLLRGYDQSAYDGKDYIALNEHLRSCTAADTAAQITQRKLEAARAAEQLRAYLEG
TCVEWLRRYLENGKETLQRAEPPKTHVTHHPLSDHEATLRCWALGFYPAEITLTWQRDGEDQ
TQDTELVETRPAGDGTFQKWAAVVVPSGQEQRYTCHMQHEGLQEPLTLRWGGKGGSCSQAAC
SNSAQGSDESLITCKA

Amino acid and nucleic acid sequences encoding β2M are also known in the art. For example, a nucleic acid sequence of a human β2M is deposited as GenBank Accession No. NM_004048.

An HLA® chain may be that of UniProtKB P61769. An exemplary HLA® chain is provided by SEQ ID NO: 23. Suitably, an HLA® chain comprises an amino acid sequence that has at least about 70%, at least about 75%, at least about 80%, at least about 85%, at least about 90%, at least about 95%, at least about 96%, at least about 97%, at least about 98%, at least about 99% or about 100% sequence identity to SEQ ID NO: 23. Suitably, an HLA® chain comprises or consists of the amino acid sequence of SEQ ID NO: 23.

(SEQ ID NO: 23)
MSRSVALAVLALLSLSGLEAIQRTPKIQVYSRHPAENGKSNFLNCYVSG
FHPSDIEVDLLKNGERIEKVEHSDLSFSKDWSFYLLYYTEFTPTEKDEY
ACRVNHVTLSQPKIVKWDRDM

The lentiviral vector of the present invention may be a CD47^{high}/MHC-I^{free} lentiviral vector or a CD47^{high}/MHC-I^{low} lentiviral vector. In preferred embodiments, the lentiviral vector of the present invention is a CD47^{high}/MHC-I^{free} lentiviral vector.

The lentiviral vector of the present invention may be obtained from a CD47^{high}/MHC-I^{free} producer cell or a CD47^{high}/MHC-I^{low} producer cell. In preferred embodiments, the lentiviral vector of the present invention is obtained from a CD47^{high}/MHC-I^{free} producer cell.

### Reverse Orientation

In one aspect, the invention provides a lentiviral vector comprising a nucleotide sequence encoding low density lipoprotein receptor (LDLR) operably linked to a promoter, wherein the nucleotide sequence encoding LDLR and the promoter are in a reverse orientation in the lentiviral vector.

In one aspect, the invention provides a lentiviral vector comprising an expression cassette comprising a nucleotide sequence encoding low density lipoprotein receptor (LDLR) operably linked to a promoter, wherein the expression cassette is in a reverse orientation in the lentiviral vector.

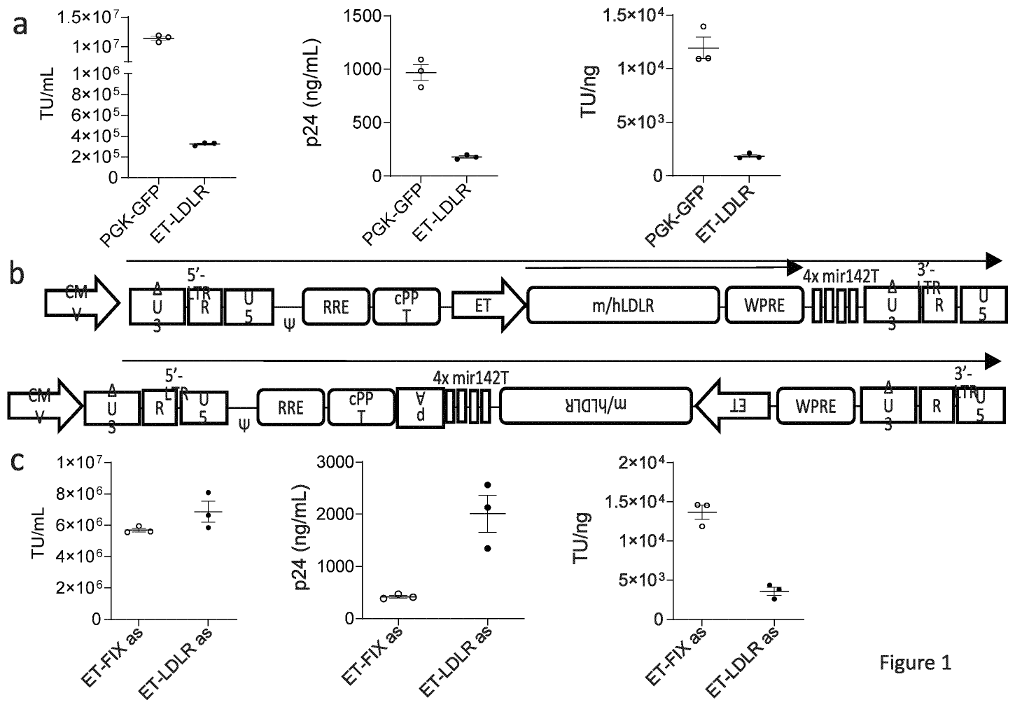

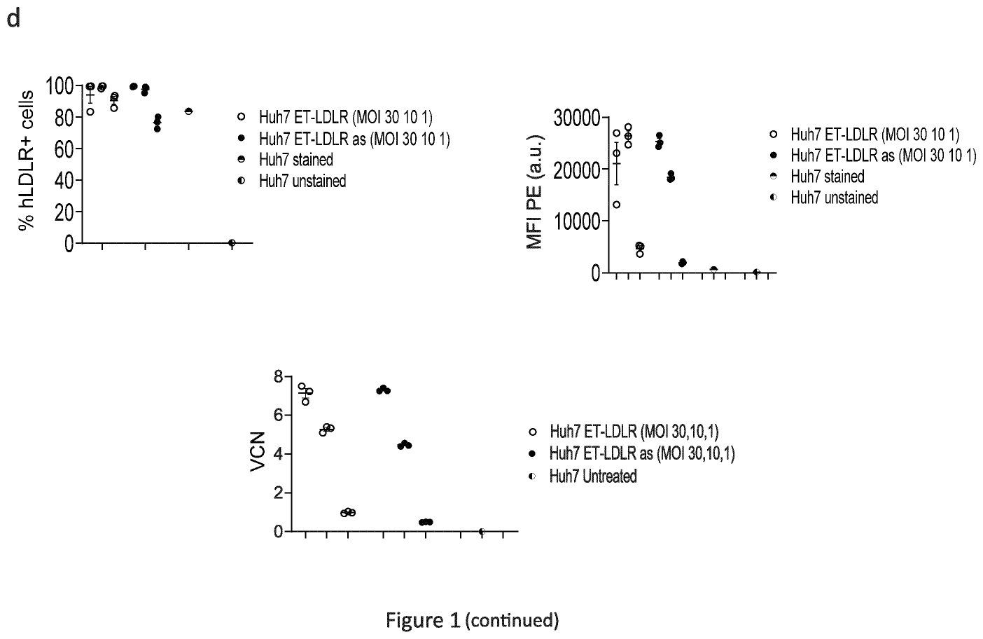

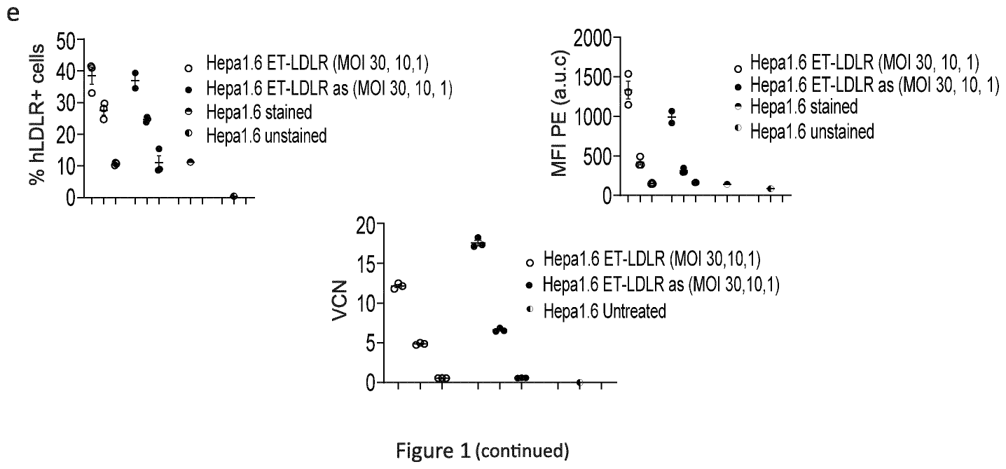

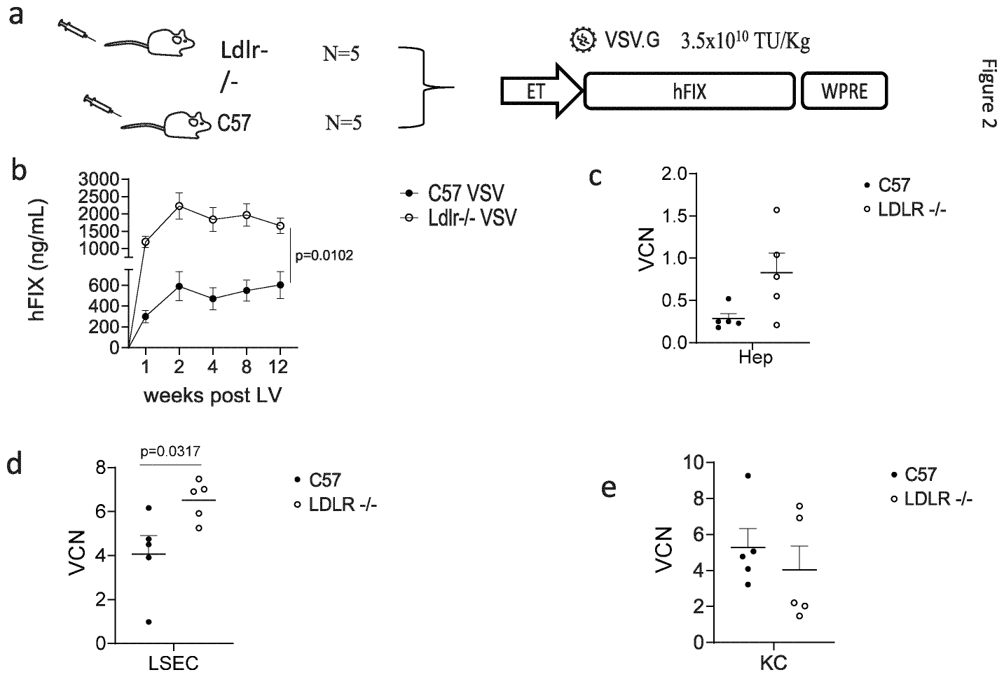

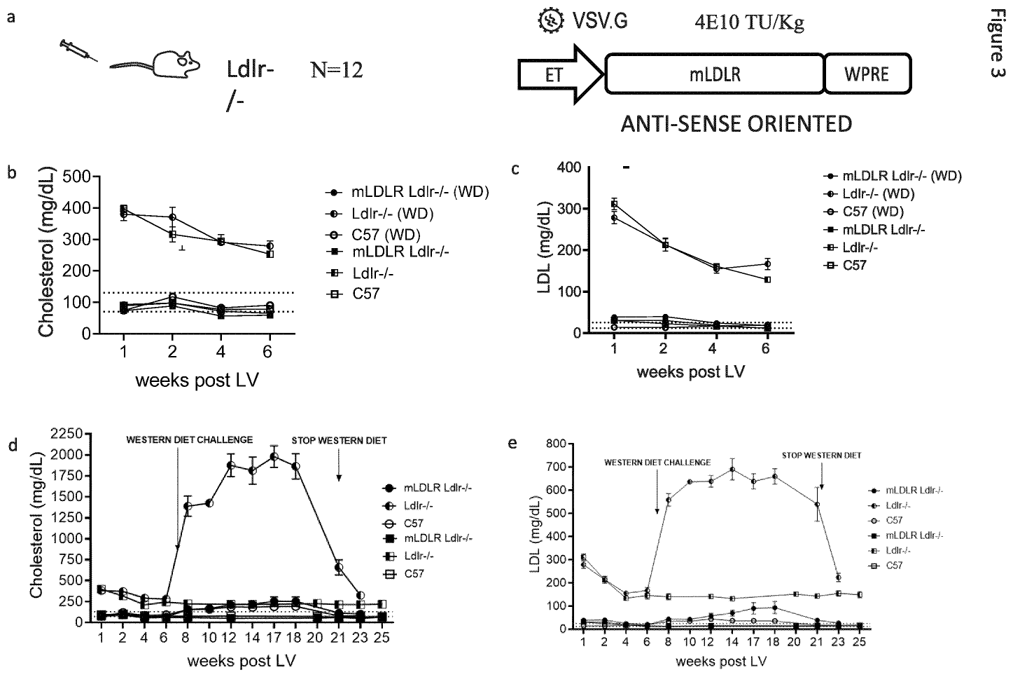

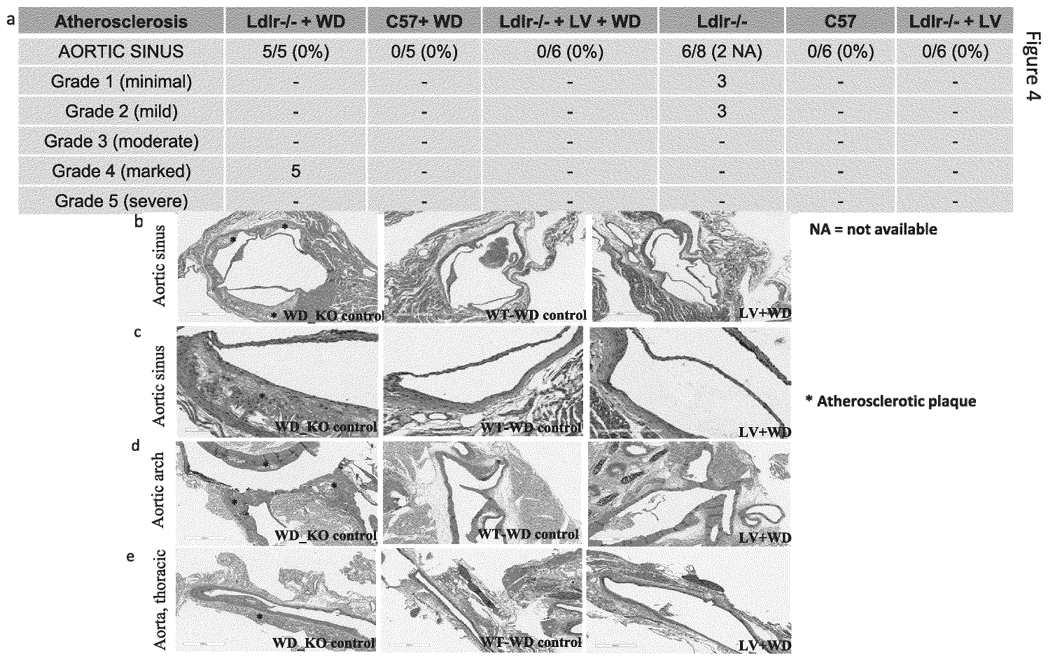

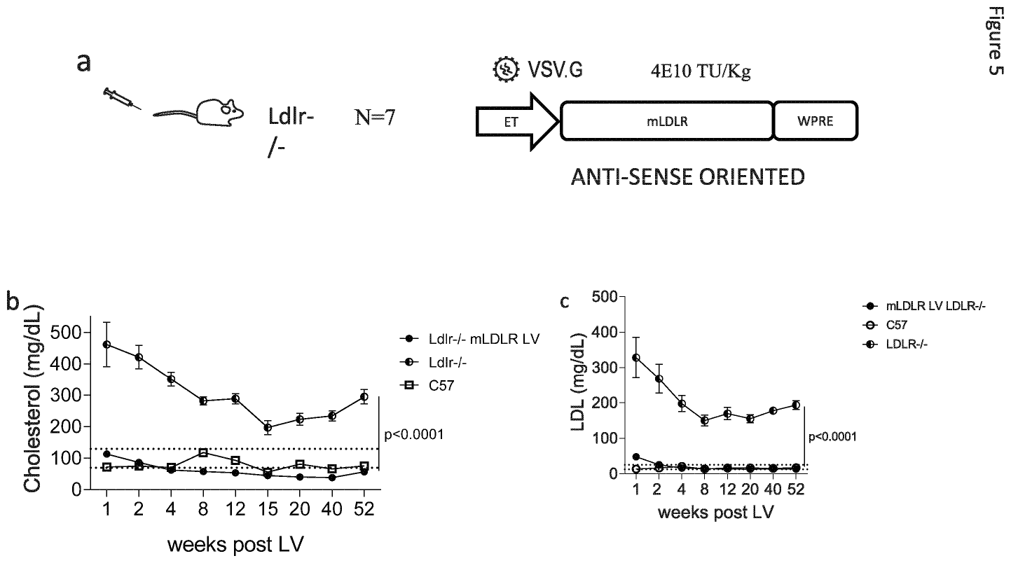

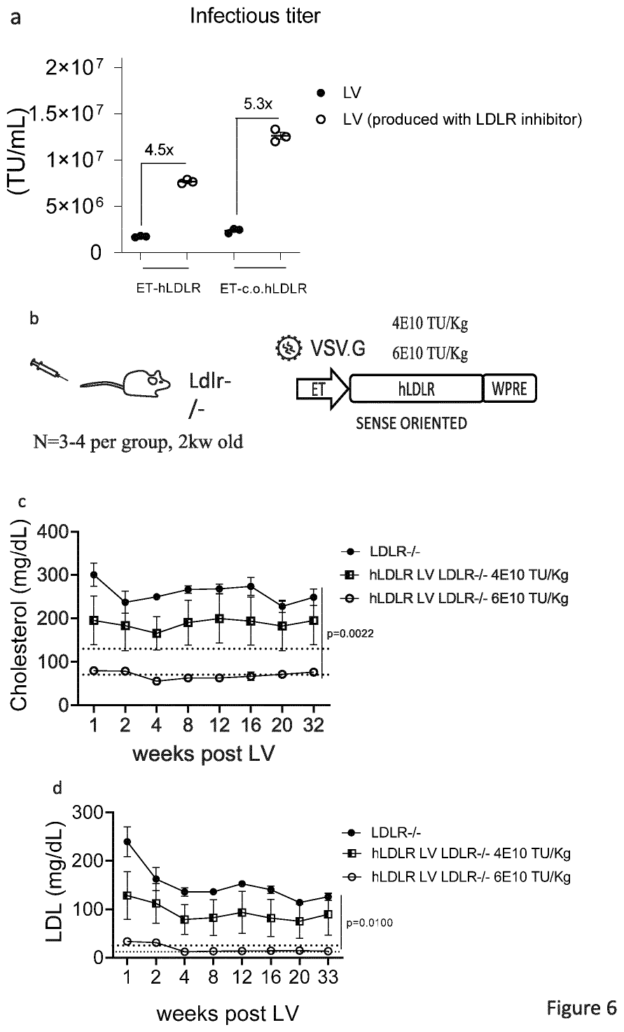

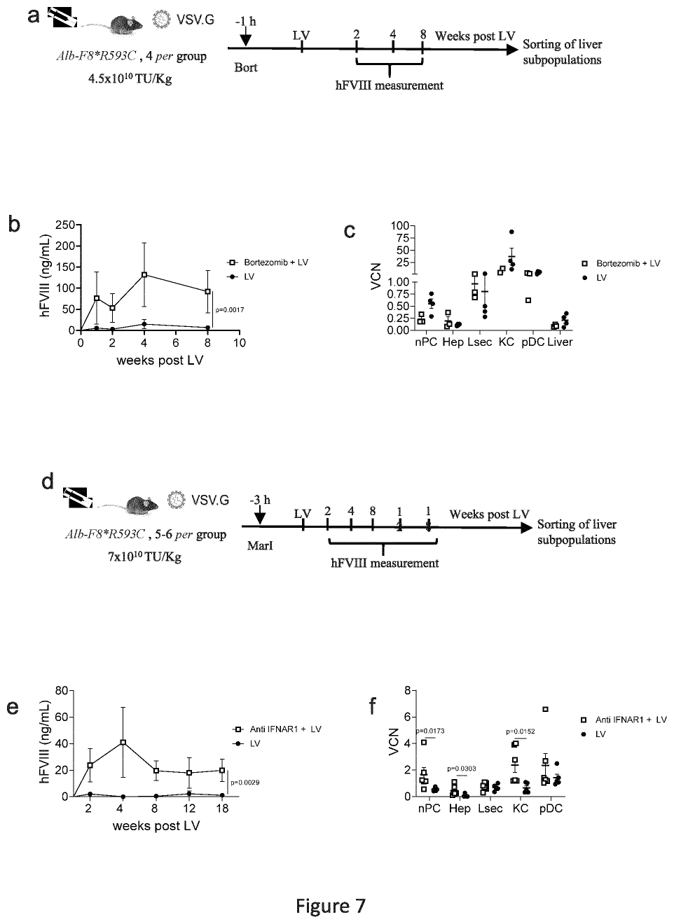

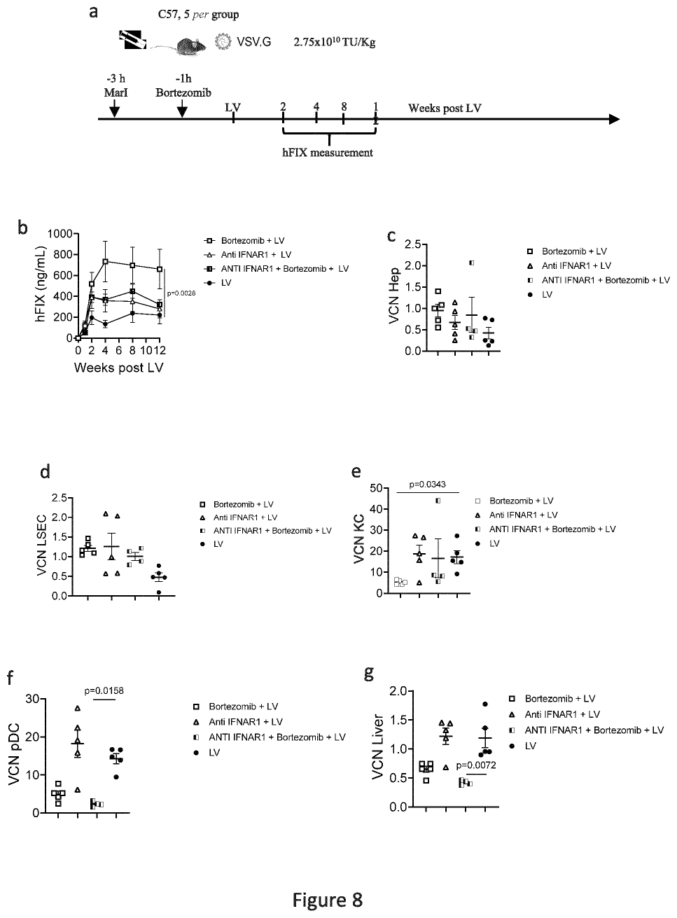

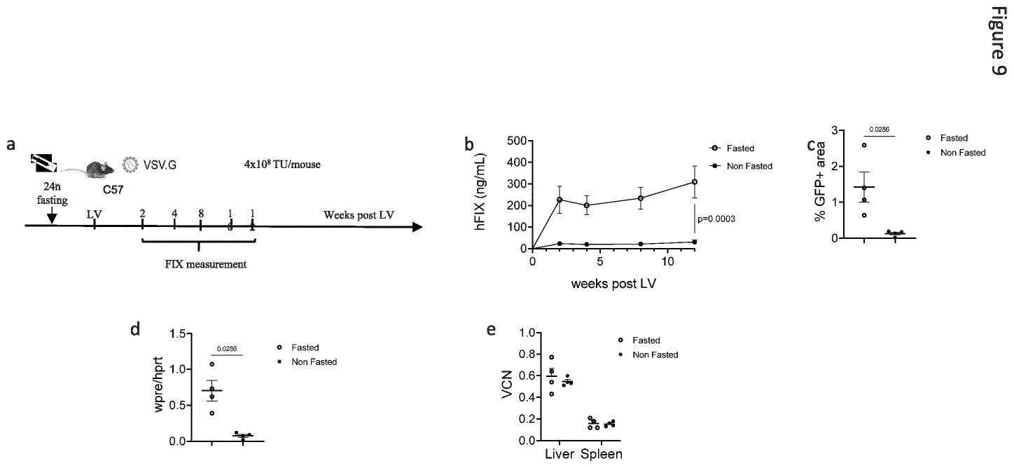

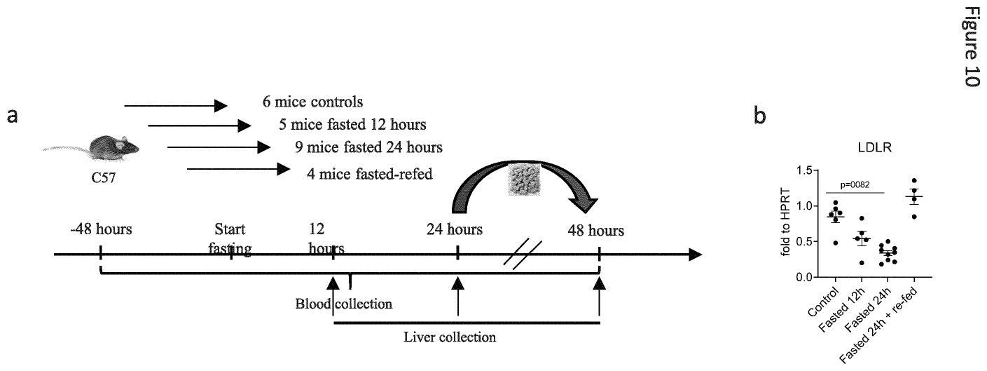

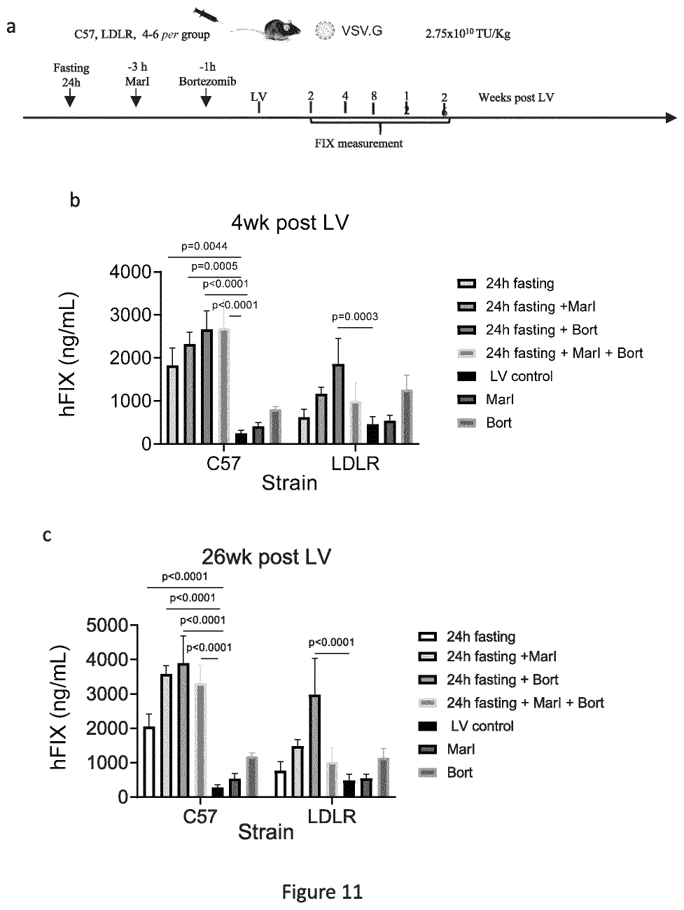

As used herein, “reverse orientation” in a lentiviral vector may refer to the nucleotide sequence encoding LDLR and the promoter, and/or the expression cassette, being incorporated into the lentiviral vector such that the direction of transcription from the promoter is oriented towards a 5′ long terminal repeat (LTR) of the lentiviral vector. The nucleotide sequence encoding LDLR or the expression cassette may be in an antisense orientation compared to transcription of the lentiviral vector genome, for example which may occur in LV producer cells during vector production (see FIG. 1).

The reverse orientation may allow transcription of the nucleotide sequence encoding LDLR from the promoter when the lentiviral vector is in a cell in which the promoter is active. The reverse orientation may prevent transcription of the nucleotide sequence encoding LDLR from a second promoter that is upstream of the 5′LTR. The second promoter may, for example, only be present in the plasmid transcribing the LV genome in producer cells during vector production.

### Regulatory Elements

The lentiviral vector of the present invention may further comprise one or more regulatory elements which may act pre- or post-transcriptionally. Suitably, the LDLR-coding sequence is operably linked to one or more regulatory elements which may act pre- or post-transcriptionally. The one or more regulatory elements may facilitate expression of the LDLR in liver cells (e.g. hepatocytes).

As used herein, a “regulatory element” may refer any nucleotide sequence that facilitates expression of a polypeptide, for example acts to increase expression of a transcript or to enhance mRNA stability. Suitable regulatory elements include for example promoters, enhancer elements, post-transcriptional regulatory elements, polyadenylation sites and Kozak sequences.

### Promoter

The lentiviral vector of the present invention may comprise a promoter, preferably a liver-specific (e.g. hepatocyte-specific) promoter. Suitably, the LDLR-coding sequence is operably linked to a promoter, preferably a liver-specific (e.g. hepatocyte-specific) promoter.

A “promoter” may refer to a region of DNA that leads to initiation of transcription of a gene. Promoters are located near the transcription start sites of genes.

As used herein, a “tissue-specific promoter” may refer to a promoter which preferentially facilitates expression of a transgene in a specific type of cells or tissue. Suitably, a tissue-specific promoter may facilitate higher expression of a transgene in one cell type as compared to other cell types. Higher expression may be measured for example by measuring the expression of a transgene, for example green fluorescence protein (GFP), operably linked to the promoter, wherein expression of the transgene correlates with the ability of the promoter to facilitate expression of a gene. For example, a tissue-specific promoter may be a promoter which facilitates transgene expression levels at least 10% higher, at least 20% higher, at least 30% higher, at least 40% higher, at least 50% higher, at least 100% higher, at least 200% higher, at least 300% higher, at least 400% higher, at least 500% higher or at least 1000% higher in one cell type as compared to expression levels in other cell types.

In some embodiments, the promoter is a liver-specific promoter. In some embodiments, the promoter is a hepatocyte-specific promoter.

Suitably, the promoter may be (or may be derived from) a promoter associated with a gene with selective expression in human liver cells (e.g. hepatocytes). Suitably, the promoter may be (or may be derived from) a promoter associated with a gene with selective expression in human hepatocyte cells. Methods to identify promoters associated with genes will be well known to the skilled person.

Exemplary liver-specific and/or hepatocyte-specific promoters are described in Kattenhorn, et al. (2016) Human Gene Therapy 27: 947-961 and include transthyretin (TTR) promoters, alpha-1-antityrpsin (AAT) promoters, thyroxine-binding globulin (TBG) promoters, APoE/hAAT promoters, HCR-hAAT promoters, LP1 promoters and HLP promoters.

An engineered promoter variant derived from any of these promoters may be used, provided that the variant retains the capacity to drive liver-specific and/or hepatocyte-specific expression of a transgene which is operably coupled to the promoter. A skilled person will be able to arrive at such variants using methods known in the art. The variant may, for example, have at least 70%, at least 80%, at least 90%, at least 95%, at least 96%, at least 97%, at least 98% or at least 99% sequence identity to any of the promoters.

A fragment of any of these promoters (or variants thereof) may be used, provided that the fragment retains the capacity to drive liver-specific and/or hepatocyte-specific expression of a transgene which is operably coupled to the promoter. A skilled person will be able to arrive at such fragments using methods known in the art. The fragment may be, for example, at least 200 nucleotides, at least 300 nucleotides, at least 400 nucleotides, at least 500 nucleotides or at least 1000 nucleotides in length.

In some embodiments, the promoter is selected from the group consisting of: a transthyretin (TTR) promoter, an alpha-1-antityrpsin (AAT) promoter, a thyroxine-binding globulin (TBG) promoter, an APoE/hAAT promoter, a HCR-hAAT promoter, a LP1 promoter and a HLP promoter.

In some embodiments, the promoter is a TTR promoter, or a variant and/or fragment thereof. In some embodiments, the promoter is an enhanced TTR (ET) promoter, or a variant and/or fragment thereof.

In some embodiments, the ET promoter comprises or consists of a nucleotide sequence that has at least 70% sequence identity to SEQ ID NO: 24 or a fragment thereof. Suitably, the ET promoter comprises or consists of a nucleotide sequence that has at least 75%, at least 80%, at least 85%, at least 90%, at least 95%, at least 96%, at least 97%, at least 98%, or at least 99% sequence identity to SEQ ID NO: 24 or a fragment thereof.

In some embodiments, the ET promoter comprises or consists of the nucleotide sequence SEQ ID NO: 24 or a fragment thereof.

(SEQ ID NO: 24)
CGCGAGTTAATAATTACCAGCGCGGGCCAAATAAATAATCCGCGAGGGGCAGGTGACGTTTG
CCCAGCGCGCGCTGGTAATTATTAACCTCGCGAATATTGATTCGAGGCCGCGATTGCCGCAA
TCGCGAGGGGCAGGTGACCTTTGCCCAGCGCGCGTTCGCCCCGCCCCGGACGGTATCGATAA
GCTTAGGAGCTTGGGCTGCAGGTCGAGGGCACTGGGAGGATGTTGAGTAAGATGGAAAACTA
CTGATGACCCTTGCAGAGACAGAGTATTAGGACATGTTTGAACAGGGGCCGGGCGATCAGCA
GGTAGCTCTAGAGGATCCCCGTCTGTCTGCACATTTCGTAGAGCGAGTGTTCCGATACTCTA
ATCTCCCTAGGCAAGGTTCATATTTGTGTAGGTTACTTATTCTCCTTTTGTTGACTAAGTCA
ATAATCAGAATCAGCAGGTTTGGAGTCAGCTTGGCAGGGATCAGCAGCCTGGGTTGGAAGGA
GGGGGTATAAAAGCCCCTTCACCAGGAGAAGCCGTCACACAGATCCACAAGCTCCTG

In some embodiments, the promoter is an AAT promoter, or a variant and/or fragment thereof. In some embodiments, the promoter is a human AAT (hAAT) promoter, or a variant and/or fragment thereof.

In some embodiments, the hAAT promoter comprises or consists of a nucleotide sequence that has at least 70% sequence identity to SEQ ID NO: 25 or a fragment thereof. Suitably, the hAAT promoter comprises or consists of a nucleotide sequence that has at least 75%, at least 80%, at least 85%, at least 90%, at least 95%, at least 96%, at least 97%, at least 98% or at least 99% sequence identity to SEQ ID NO: 25 or a fragment thereof.

In some embodiments, the hAAT promoter comprises or consists of the nucleotide sequence SEQ ID NO: 25 or a fragment thereof.

(SEQ ID NO: 25)
GATCTTGCTACCAGTGGAACAGCCACTAAGGATTCTGCAGTGAGAGCAG
AGGGCCAGCTAAGTGGTACTCTCCCAGAGACTGTCTGACTCACGCCACC
CCCTCCACCTTGGACACAGGACGCTGTGGTTTCTGAGCCAGGTACAATG
ACTCCTTTCGGTAAGTGCAGTGGAAGCTGTACACTGCCCAGGCAAAGCG
TCCGGGCAGCGTAGGCGGGCGACTCAGATCCCAGCCAGTGGACTTAGCC
CCTGTTTGCTCCTCCGATAACTGGGGTGACCTTGGTTAATATTCACCAG
CAGCCTCCCCCGTTGCCCCTCTGGATCCACTGCTTAAATACGGACGAGG
ACAG

The promoter may be a constitutive promoter. As used herein, a “constitutive promoter” is a promoter which is always active.

Alternatively, the promoter may be an inducible promoter. As used herein, an “inducible promoter” is a promoter which is only active under specific conditions. For example, expression of the transgene may be induced by a small molecule or drug (e.g. which binds to a promoter, regulatory sequence or to a transcriptional repressor or activator molecule) or by using an environmental trigger. Types of inducible promoter include chemically-inducible promoters (e.g. a Tet-on system); temperature-inducible promoters (e.g. Hsp70 or Hsp90-derived promoters); and light-inducible promoters. Suitably, the promoter is chemically-inducible. Any suitable method for engineering an inducible promoter may be used.

### Enhancer Elements

The lentiviral vector of the present invention may comprise an enhancer, preferably a liver-specific (e.g. hepatocyte-specific) enhancer. Suitably, the LDLR-coding sequence is operably linked to an enhancer, preferably a liver-specific (e.g. hepatocyte-specific) enhancer.

An “enhancer” or “enhancer element” may refer a region of DNA that can be bound by proteins (activators) to increase the likelihood that transcription of a particular gene will occur. Enhancers are cis-acting. They can be located up to 1 Mbp (1,000,000 bp) away from the gene, upstream or downstream from the start site.

As used herein, a “tissue-specific enhancer” is an enhancer which preferentially facilitates expression of a gene in specific cells or tissues. Suitably, a tissue-specific enhancer may facilitate higher expression of a gene in specific cells types as compared to other cell types. Higher expression may be measured for example by measuring the expression of a transgene, for example green fluorescence protein (GFP), operably linked to the enhancer, wherein expression of the transgene correlates with the ability of the enhancer to facilitate expression of a gene. For example, a tissue-specific enhancer may be an enhancer which facilitates gene expression levels at least 10% higher, at least 20% higher, at least 30% higher, at least 40% higher, at least 50% higher, at least 100% higher, at least 200% higher, at least 300% higher, at least 400% higher, at least 500% higher or at least 1000% higher in a specific cell-type compared to expression levels in other cell types.

Suitable tissue-specific enhancers will be well known to the skilled person. The enhancer may be a liver-specific enhancer, preferably a hepatocyte-specific enhancer.

Suitably, the enhancer may be (or may be derived from) an enhancer associated with a gene with selective expression in human liver cells (e.g. hepatocytes). Suitably, the enhancer may be (or may be derived from) an enhancer associated with a gene with selective expression in human hepatocyte cells. Methods to identify the enhancer regions associated with genes will be well known to the skilled person.

Exemplary liver-specific and/or hepatocyte-specific enhancers are described in Kramer et al. (2003) Molecular Therapy 7: 375-385, and include enhancer regions of the albumin, α1-antitrypsin, hepatitis B virus core protein, and hemopexin genes. Other liver-specific and/or hepatocyte-specific enhancers include apolipoprotein E (APoE) enhancers, hepatic control region (HCR) enhancers and alpha-1-antitrypsin (AAT) enhancers.

An engineered enhancer variant derived from any of these enhancers may be used, provided that the variant retains the capacity to drive liver-specific and/or hepatocyte-specific expression of a transgene which is operably coupled to the enhancer. A skilled person will be arrive at such variants using methods known in the art. The variant may have at least 70%, at least 80%, at least 90%, at least 95%, at least 96%, at least 97%, at least 98% or at least 99% sequence identity to any of the enhancers.

A fragment of any of these enhancers (or variants thereof) may be used, provided that the fragment retains the capacity to drive liver-specific and/or hepatocyte-specific expression of a transgene which is operably coupled to the enhancer. A skilled person will be able to arrive at such fragments using methods known in the art. The fragment may be at least 200 nucleotides, at least 300 nucleotides, at least 400 nucleotides, at least 500 nucleotides or at least 1000 nucleotides in length.

The vector of the present invention may comprise a liver-specific promoter and/or a liver-specific enhancer, i.e. a liver-specific promoter and/or enhancer. Suitably, the LDLR-coding sequence is operably linked to a liver-specific promoter and/or enhancer. Suitably, the LDLR-coding sequence is operably linked to a hepatocyte-specific promoter and/or enhancer. The promoter and enhancer may be a combination of any of the above, for example a hAAT promoter and an ApoE or HCR enhancer.

### Post-Transcriptional Regulatory Elements

The lentiviral vector of the present invention may comprise one or more further post-transcriptional regulatory elements (e.g. in addition to one or more miRNA target sequence). Suitably, the protein-coding sequence is operably linked to one or more further post-transcriptional regulatory elements. The further post-transcriptional regulatory element may improve gene expression.

The lentiviral vector of the present invention may comprise a Woodchuck Hepatitis Virus Post-transcriptional Regulatory Element (WPRE). Suitably, the LDLR-coding sequence is operably linked to a WPRE.

Suitable WPRE sequences will be well known to those of skill in the art (see, for example, Zufferey et al. (1999) Journal of Virology 73: 2886-2892; Zanta-Boussif et al. (2009) Gene Therapy 16: 605-619). Suitably, the WPRE is a wild-type WPRE or is a mutant WPRE. For example, the WPRE may be mutated to abrogate translation of the woodchuck hepatitis virus X protein (WHX), for example by mutating the WHX ORF translation start codon.

In some embodiments, the WPRE comprises or consists of a nucleotide sequence that has at least 70% sequence to SEQ ID NO: 26 or a fragment thereof. Suitably, the WPRE comprises or consists of a nucleotide sequence that has at least 75%, at least 80%, at least 85%, at least 90%, at least 95%, at least 96%, at least 97%, at least 98% or at least 99% sequence identity to SEQ ID NO: 26 or a fragment thereof.

In some embodiments, the WPRE comprises or consists of the nucleotide sequence SEQ ID NO: 26 or a fragment thereof.

(SEQ ID NO: 26)
AATCAACCTCTGGATTACAAAATTTGTGAAAGATTGACTGGTATTCTTA
ACTATGTTGCTCCTTTTACGCTATGTGGATACGCTGCTTTAATGCCTTT
GTATCATGCTATTGCTTCCCGTATGGCTTTCATTTTCTCCTCCTTGTAT
AAATCCTGGTTGCTGTCTCTTTATGAGGAGTTGTGGCCCGTTGTCAGGC
AACGTGGCGTGGTGTGCACTGTGTTTGCTGACGCAACCCCCACTGGTTG
GGGCATTGCCACCACCTGTCAGCTCCTTTCCGGGACTTTCGCTTTCCCC
CTCCCTATTGCCACGGCGGAACTCATCGCCGCCTGCCTTGCCCGCTGCT
GGACAGGGGCTCGGCTGTTGGGCACTGACAATTCCGTGGTGTTGTCGGG
GAAATCATCGTCCTTTCCTTGGCTGCTCGCCTGTGTTGCCACCTGGATT
CTGCGCGGGACGTCCTTCTGCTACGTCCCTTCGGCCCTCAATCCAGCGG
ACCTTCCTTCCCGC

### Polyadenylation Sequence

The lentiviral vector of the present invention may comprise a polyadenylation sequence. Suitably, the LDLR-coding sequence is operably linked to a polyadenylation sequence. A polyadenylation sequence may be inserted after the LDLR-coding sequence to improve transgene expression.

A polyadenylation sequence typically comprises a polyadenylation signal, a polyadenylation site and a downstream element: the polyadenylation signal comprises the sequence motif recognised by the RNA cleavage complex; the polyadenylation site is the site of cleavage at which a poly-A tails is added to the mRNA; the downstream element is a GT-rich region which usually lies just downstream of the polyadenylation site, which is important for efficient processing.

Suitable polyadenylation sequences will be well known to those of skill in the art (see, for example, Schambach et al. (2007) Molecular Therapy 15: 1167-1173; Choi et al. (2014) Molecular Brain 7: 1-10). Exemplary polyadenylation sequences include the bGH poly(A) signal sequence and SV40 pA signal sequence.

### Kozak Sequence

The lentiviral vector of the present invention may comprise a Kozak sequence. Suitably, the LDLR-coding sequence is operably linked to a Kozak sequence. A Kozak sequence may be inserted before the start codon to improve the initiation of translation.

Suitable Kozak sequences will be well known to the skilled person (see, for example, Kozak (1987) Nucleic Acids Research 15: 8125-8148).

In some embodiments, the Kozak sequence comprises or consists of a nucleotide sequence that has at least 80% sequence identity to SEQ ID NO: 27 or a fragment thereof.

In some embodiments, the Kozak sequence comprises or consists of the nucleotide sequence SEQ ID NO: 27 or a fragment thereof.

(SEQ ID NO: 27)
GCCACC

### Other Cis-Acting Elements

The lentiviral vector of the present invention may comprise any other suitable cis-acting elements, such as one or more of a rev response element (RRE); a retroviral psi packaging element; a primer binding site (PBS); a TAT activation region (TAR); splice donor and acceptor sites; and central and terminal polypurine tracts.

### Long Terminal Repeats (LTRs)

The lentiviral vector of the present invention may comprise one or more long terminal repeat (LTR). LTRs are responsible for proviral integration and transcription. Typically, a naturally occurring LTR comprises U3, R, and U5 regions.

The lentiviral vector may comprise a 5′ LTR and/or a 3′ LTR. The lentiviral vector may comprise a 5′ LTR and a 3′ LTR. Suitably, a 5′ LTR comprises R and U5 regions, and optionally comprises a U3 region. Suitably, a 3′ LTR comprises U3, R and U5 regions.

Suitable LTR sequences will be well known to the skilled person (see, for example, Frech et al. (1996) Virology 224: 256-267).

In some embodiments, a LTR comprises or consists of a nucleotide sequence that has at least 70% sequence identity to SEQ ID NO: 28 or a fragment thereof. Suitably, a LTR comprises or consists of a nucleotide sequence that has at least 75%, at least 80%, at least 85%, at least 90%, at least 95%, at least 96%, at least 97%, at least 98% or at least 99% sequence identity to SEQ ID NO: 28 or a fragment thereof.

In some embodiments, a LTR comprises or consists of the nucleotide sequence SEQ ID NO: 28 or a fragment thereof.

(SEQ ID NO: 28)
TGGAAGGGCTAATTCACTCCCAACGAAGACAAGATCTGCTTTTTGCTTG
TACTGGGTCTCTCTGGTTAGACCAGATCTGAGCCTGGGAGCTCTCTGGC
TAACTAGGGAACCCACTGCTTAAGCCTCAATAAAGCTTGCCTTGAGTGC
TTCAAGTAGTGTGTGCCCGTCTGTTGTGTGACTCTGGTAACTAGAGATC
CCTCAGACCCTTTTAGTCAGTGTGGAAAATCTCTAGCAG

The lentiviral vector of the present invention may comprise one or more self-inactivating long terminal repeat (SIN-LTR). A “SIN-LTR” may comprise a deletion that abolishes transcription of the full-length virus after it has incorporated into a host cell. For example, a 3′ SIN-LTR may comprise a deletion in the U3 region removing the promoter/enhancer elements (see, for example, Zufferey et al. (1998) Journal of Virology 72: 9873-9880). This deletion is copied into the 5′ LTR after reverse transcription, thereby making the gene expression in target cells dependent on an internal promoter of choice.

Suitable SIN-LTR sequences will be well known to the skilled person (see, for example, Zufferey et al. (1998) Journal of Virology 72: 9873-9880; Miyoshi et al. (1998) Journal of Virology 72: 8150-8157).

In some embodiments, the 5′ LTR comprises or consists of a nucleotide sequence that has at least 70% sequence identity to SEQ ID NO: 29 or a fragment thereof. Suitably, the 5′ LTR comprises or consists of a nucleotide sequence that has at least 75%, at least 80%, at least 85%, at least 90%, at least 95%, at least 96%, at least 97%, at least 98% or at least 99% sequence identity to SEQ ID NO: 29 or a fragment thereof.

In some embodiments, the 5′ LTR comprises or consists of the nucleotide sequence SEQ ID NO: 29 or a fragment thereof.

(SEQ ID NO: 29)
GGGTCTCTCTGGTTAGACCAGATCTGAGCCTGGGAGCTCTCTGGCTAAC
TAGGGAACCCACTGCTTAAGCCTCAATAAAGCTTGCCTTGAGTGCTTCA
AGTAGTGTGTGCCCGTCTGTTGTGTGACTCTGGTAACTAGAGATCCCTC
AGACCCTTTTAGTCAGTGTGGAAAATCTCTAGCAG

In some embodiments, the 5′ LTR and/or the 3′ LTR comprises or consists of a nucleotide sequence that has at least 70% sequence identity to SEQ ID NO: 28 or a fragment thereof. Suitably, the 5′ LTR and/or the 3′ LTR comprises or consists of a nucleotide sequence that has at least 75%, at least 80%, at least 85%, at least 90%, at least 95%, at least 96%, at least 97%, at least 98% or at least 99% sequence identity to SEQ ID NO: 28 or a fragment thereof.

In some embodiments, the 5′ LTR and/or the 3′ LTR comprises or consists of the nucleotide sequence SEQ ID NO: 28 or a fragment thereof.

In some embodiments, the 5′ LTR and the 3′ LTR comprise or consist of a nucleotide sequence that has at least 70% sequence identity to SEQ ID NO: 28 or a fragment thereof. Suitably, the 5′ LTR and the 3′ LTR comprise or consist of a nucleotide sequence that has at least 75%, at least 80%, at least 85%, at least 90%, at least 95%, at least 96%, at least 97%, at least 98% or at least 99% sequence identity to SEQ ID NO: 28 or a fragment thereof.

In some embodiments, the 5′ LTR and the 3′ LTR comprise or consist of the nucleotide sequence SEQ ID NO: 28 or a fragment thereof.

### Primer Binding Site (PBS)

The lentiviral vector of the present invention may comprise a primer binding site (PBS). A PBS is a cis-acting element where a primer may bind to initiate reverse transcription of the RNA genome (see, for example, Lanchy et al. (1998) Journal of Biological Chemistry 273: 24425-24432).

Suitable retroviral PBSs will be well known to the skilled person.

In some embodiments, a PBS comprises or consists of a nucleotide sequence that has at least 70% sequence identity to SEQ ID NO: 30 or a fragment thereof. Suitably, a PBS comprises or consists of a nucleotide sequence that has at least 75%, at least 80%, at least 85%, at least 90%, at least 95%, at least 96%, at least 97%, at least 98% or at least 99% identical to SEQ ID NO: 30 or a fragment thereof.

In some embodiments, a PBS comprises or consists of the nucleotide sequence SEQ ID NO: or a fragment thereof.

(SEQ ID NO: 30)
TGGCGCCCGAACAGGGACTTGAAAGCGAAAGGGAAACCAGAGGAGCTCT
CTCGACGCAGGACTCGGCTTGCTGAAGCGCGCACGGCAAGAGGCGAGGG
GCGGCGACTGGTGAGTACGCCAAAAATTTTGACTAGCGGAGGCTAGAAG
GAGAGAG

### Retroviral Psi Packaging Element

The lentiviral vector of the present invention may comprise a retroviral psi packaging element. A retroviral psi packaging element is a cis-acting element which is involved in regulating the process of packaging the retroviral RNA genome into the viral capsid during replication (see, for example, McBride et al. (1997) Journal of Virology 71: 4544-4554). A retroviral psi packaging element may form part of the 5′ region of the gag gene.

Suitable retroviral psi packaging elements will be well known to the skilled person.

In some embodiments, a retroviral psi packaging element comprises or consists of a nucleotide sequence that has at least 70% sequence identity to SEQ ID NO: 31 or a fragment thereof. Suitably, a retroviral psi packaging element comprises or consists of a nucleotide sequence that has at least 75%, at least 80%, at least 85%, at least 90%, at least 95%, at least 96%, at least 97%, at least 98% or at least 99% sequence identity to SEQ ID NO: 31 or a fragment thereof.

In some embodiments, a retroviral psi packaging element comprises or consists of the nucleotide sequence SEQ ID NO: 31 or a fragment thereof.

(SEQ ID NO: 31)
ATGGGTGCGAGAGCGTCAGTATTAAGCGGGGGAGAATTAGATCGCGATG
GGAAAAAATTCGGTTAAGGCCAGGGGGAAAGAAAAAATATAAATTAAAA
CATATAGTATGGGCAAGCAGGGAGCTAGAACGATTCGCAGTTAATCCTG
GCCTGTTAGAAACATCAGAAGGCTGTAGACAAATACTGGGACAGCTACA
ACCATCCCTTCAGACAGGATCAGAAGAACTTAGATCATTATATAATACA
GTAGCAACCCTCTATTGTGTGCATCAAAGGATAGAGATAAAAGACACCA
AGGAAGCTTTAGACAAGATAGAGGAAGAGCAAAACAAAAGTAAGACCAC
CGCACAGCAAGCGGCCGCTGAT

### Rev Response Element (RRE)

The lentiviral vector of the present invention may comprise a rev response element (RRE). A RRE is a cis-acting element that enables the efficient export of RNA transcripts of the integrated provirus from the nucleus to the cytoplasm of an infected target cell (see, for example, Pollard et al. (1998) Annual Review of Microbiology 52: 491-532).

Suitable RRE sequences will be well known to the skilled person.

In some embodiments, a RRE comprises or consists of a nucleotide sequence that has at least 70% sequence identity to SEQ ID NO: 33 or a fragment thereof. Suitably, a RRE comprises or consists of a nucleotide sequence that has at least 75%, at least 80%, at least 85%, at least 90%, at least 95%, at least 96%, at least 97%, at least 98% or at least 99% identical to SEQ ID NO: 33 or a fragment thereof.

In some embodiments, a RRE comprises or consists of the nucleotide sequence SEQ ID NO: 33 or a fragment thereof.

(SEQ ID NO: 33)
GGAGCTTTGTTCCTTGGGTTCTTGGGAGCAGCAGGAAGCACTATGGGCG
CAGCGTCAATGACGCTGACGGTACAGGCCAGACAATTATTGTCTGGTAT
AGTGCAGCAGCAGAACAATTTGCTGAGGGCTATTGAGGCGCAACAGCAT
CTGTTGCAACTCACAGTCTGGGGCATCAAGCAGCTCCAGGCAAGAATCC
TGGCTGTGGAAAGATACCTAAAGGATCAACAGCTCCTGGGGATTT

Central Polypurine Tract (cPPT)

The lentiviral vector of the present invention may comprise a central polypurine tract (cPPT). A cPPT may allow initiation of plus-strand synthesis (see, for example, Follenzi et al. (2000) Nature Genetics 25: 217-222).

Suitable cPPT sequences will be well known to the skilled person.

In some embodiments, a cPPT comprises or consists of a nucleotide sequence that has at least 70% sequence identity to SEQ ID NO: 34 or a fragment thereof. Suitably, a cPPT comprises or consists of a nucleotide sequence that has at least 75%, at least 80%, at least 85%, at least 90%, at least 95%, at least 96%, at least 97%, at least 98% or at least 99% sequence identity to SEQ ID NO: 34 or a fragment thereof.

In some embodiments, a cPPT comprises or consists of the nucleotide sequence SEQ ID NO: 34 or a fragment thereof.

(SEQ ID NO: 34)
AACTTTTAAAAGAAAAGGGGGGATTGGGGGGTACAGTGCAGGGGAAAGA
ATAGTAGACATAATAGCAACAGACATACAAACTAAAGAATTACAAAAAC
AAATTACAAAAATTCAAAATTTTATC

### Other Elements

The lentiviral vector of the present invention may comprise any other suitable elements.

In some embodiments, the lentiviral vector of the present invention comprises an element comprising or consisting of a nucleotide sequence that has at least 70%, at least 75%, at least 80%, at least 85%, at least 90%, at least 95%, at least 96%, at least 97%, at least 98% or at least 99% sequence identity to SEQ ID NO: 35 or a fragment thereof. In some embodiments, the lentiviral vector of the present invention comprises an element comprising or consisting of the nucleotide sequence of SEQ ID NO: 35 or a fragment thereof.

(SEQ ID NO: 3-Exemplary delta ENV1)
TCTTCAGACCTGGAGGAGGAGATATGAGGGACAATTGGAGAAGTGAATT
ATATAAATATAAAGTAGTAAAAATTGAACCATTAGGAGTAGCACCCACC
AAGGCAAAGAGAAGAGTGGTGCAGAGAGAAAAAAGAGCAGTGGGAATA

In some embodiments, the lentiviral vector of the present invention comprises an element comprising or consisting of a nucleotide sequence that has at least 70%, at least 75%, at least 80%, at least 85%, at least 90%, at least 95%, at least 96%, at least 97%, at least 98% or at least 99% sequence identity to SEQ ID NO: 36 or a fragment thereof. In some embodiments, the lentiviral vector of the present invention comprises an element comprising or consisting of the nucleotide sequence of SEQ ID NO: 36 or a fragment thereof.

(SEQ ID NO: 36-Exemplary delta ENV2)
GGGTTGCTCTGGAAAACTCATTTGCACCACTGCTGTGCCTTGGAATGCTA
GTTGGAGTAATAAATCTCTGGAACAGATTTGGAATCACACGACCTGGATG
GAGTGGGACAGAGAAATTAACAATTACACAAGCTTAATACACTCCTTAAT
TGAAGAATCGCAAAACCAGCAAGAAAAGAATGAACAAGAATTATTGGAAT
TAGATAAATGGGCAAGTTTGTGGAATTGGTTTAACATAACAAATTGGCTG
TGGTATATAAAATTATTCATAATGATAGTAGGAGGCTTGGTAGGTTTAAG
AATAGTTTTTGCTGTACTTTCTATAGTGAATAGAGTTAGGCAGGGATATT
CACCATTATCGTTTCAGACCCACCTCCCAACCCCGAGGGGACCCGACAGG
CCCGAAGGAATAGAAGAAGAAGGTGGAGAGAGAGACAGAGACAGATCCAT
TCGATTAGTGAACGGATC

### Exemplary Lentiviral Genomes

The lentiviral genome of the present invention may comprise from 5′ to 3′: a 5′ LTR, one or more cis-acting elements, and a 3′ LTR.

For example, the lentiviral genome of the present invention may comprise from 5′ to 3′: a 5′ LTR, a PBS, a retroviral psi packaging element, a RRE, a cPPT, an expression cassette (preferably in reverse orientation), a WPRE, and a 3′ LTR.

The expression cassette may, for example comprise a nucleotide sequence encoding LDLR operably linked to a promoter, and optionally operably linked to one or more miRNA target sequence and/or a polyadenylation sequence.

The lentiviral genome of the present invention may further comprise any other suitable elements, such as any other elements described herein or one or more spacer sequence. The spacer sequence(s) may comprise, for example, at least one (e.g. 1, 2, 3, 4, 5, 6, 7, 8, 9, 10), at least two, at least three, at least four, at least five, at least six, at least seven, at least eight, at least nine or at least ten nucleotide bases.

In some embodiments, the lentiviral genome comprises or consists of a nucleotide sequence that has at least 70% sequence identity to SEQ ID NO: 37 or a fragment thereof. Suitably, the lentiviral genome comprises or consists of a nucleotide sequence that has at least 75%, at least 80%, at least 85%, at least 90%, at least 95%, at least 96%, at least 97%, at least 98% or at least 99% sequence identity to SEQ ID NO: 37 or a fragment thereof.

In some embodiments, the lentiviral genome comprises or consists of the nucleotide sequence SEQ ID NO: 37 or a fragment thereof.

(SEQ ID NO: 37)
CAGGTGGCACTTTTCGGGGAAATGTGCGCGGAACCCCTATTTGTTTATTTTTCTAAATACAT
TCAAATATGTATCCGCTCATGAGACAATAACCCTGATAAATGCTTCAATAATATTGAAAAAG
GAAGAGTATGAGTATTCAACATTTCCGTGTCGCCCTTATTCCCTTTTTTGCGGCATTTTGCC
TTCCTGTTTTTGCTCACCCAGAAACGCTGGTGAAAGTAAAAGATGCTGAAGATCAGTTGGGT
GCACGAGTGGGTTACATCGAACTGGATCTCAACAGCGGTAAGATCCTTGAGAGTTTTCGCCC
CGAAGAACGTTTTCCAATGATGAGCACTTTTAAAGTTCTGCTATGTGGCGCGGTATTATCCC
GTATTGACGCCGGGCAAGAGCAACTCGGTCGCCGCATACACTATTCTCAGAATGACTTGGTT
GAGTACTCACCAGTCACAGAAAAGCATCTTACGGATGGCATGACAGTAAGAGAATTATGCAG
TGCTGCCATAACCATGAGTGATAACACTGCGGCCAACTTACTTCTGACAACGATCGGAGGAC
CGAAGGAGCTAACCGCTTTTTTGCACAACATGGGGGATCATGTAACTCGCCTTGATCGTTGG
GAACCGGAGCTGAATGAAGCCATACCAAACGACGAGCGTGACACCACGATGCCTGTAGCAAT
GGCAACAACGTTGCGCAAACTATTAACTGGCGAACTACTTACTCTAGCTTCCCGGCAACAAT
TAATAGACTGGATGGAGGCGGATAAAGTTGCAGGACCACTTCTGCGCTCGGCCCTTCCGGCT
GGCTGGTTTATTGCTGATAAATCTGGAGCCGGTGAGCGTGGGTCTCGCGGTATCATTGCAGC
ACTGGGGCCAGATGGTAAGCCCTCCCGTATCGTAGTTATCTACACGACGGGGAGTCAGGCAA
CTATGGATGAACGAAATAGACAGATCGCTGAGATAGGTGCCTCACTGATTAAGCATTGGTAA
CTGTCAGACCAAGTTTACTCATATATACTTTAGATTGATTTAAAACTTCATTTTTAATTTAA
AAGGATCTAGGTGAAGATCCTTTTTGATAATCTCATGACCAAAATCCCTTAACGTGAGTTTT
CGTTCCACTGAGCGTCAGACCCCGTAGAAAAGATCAAAGGATCTTCTTGAGATCCTTTTTTT
CTGCGCGTAATCTGCTGCTTGCAAACAAAAAAACCACCGCTACCAGCGGTGGTTTGTTTGCC
GGATCAAGAGCTACCAACTCTTTTTCCGAAGGTAACTGGCTTCAGCAGAGCGCAGATACCAA
ATACTGTCCTTCTAGTGTAGCCGTAGTTAGGCCACCACTTCAAGAACTCTGTAGCACCGCCT
ACATACCTCGCTCTGCTAATCCTGTTACCAGTGGCTGCTGCCAGTGGCGATAAGTCGTGTCT
TACCGGGTTGGACTCAAGACGATAGTTACCGGATAAGGCGCAGCGGTCGGGCTGAACGGGGG
GTTCGTGCACACAGCCCAGCTTGGAGCGAACGACCTACACCGAACTGAGATACCTACAGCGT
GAGCTATGAGAAAGCGCCACGCTTCCCGAAGGGAGAAAGGCGGACAGGTATCCGGTAAGCGG
CAGGGTCGGAACAGGAGAGCGCACGAGGGAGCTTCCAGGGGGAAACGCCTGGTATCTTTATA
GTCCTGTCGGGTTTCGCCACCTCTGACTTGAGCGTCGATTTTTGTGATGCTCGTCAGGGGGG
CGGAGCCTATGGAAAAACGCCAGCAACGCGGCCTTTTTACGGTTCCTGGCCTTTTGCTGGCC
TTTTGCTCACATGTTCTTTCCTGCGTTATCCCCTGATTCTGTGGATAACCGTATTACCGCCT
TTGAGTGAGCTGATACCGCTCGCCGCAGCCGAACGACCGAGCGCAGCGAGTCAGTGAGCGAG
GAAGCGGAAGAGCGCCCAATACGCAAACCGCCTCTCCCCGCGCGTTGGCCGATTCATTAATG
CAGCTGGCACGACAGGTTTCCCGACTGGAAAGCGGGCAGTGAGCGCAACGCAATTAATGTGA
GTTAGCTCACTCATTAGGCACCCCAGGCTTTACACTTTATGCTTCCGGCTCGTATGTTGTGT
GGAATTGTGAGCGGATAACAATTTCACACAGGAAACAGCTATGACCATGATTACGCCAAGCG
CGCAATTAACCCTCACTAAAGGGAACAAAAGCTGGAGCTGCAAGCTTGGCCATTGCATACGT
TGTATCCATATCATAATATGTACATTTATATTGGCTCATGTCCAACATTACCGCCATGTTGA
CATTGATTATTGACTAGTTATTAATAGTAATCAATTACGGGGTCATTAGTTCATAGCCCATA
TATGGAGTTCCGCGTTACATAACTTACGGTAAATGGCCCGCCTGGCTGACCGCCCAACGACC
CCCGCCCATTGACGTCAATAATGACGTATGTTCCCATAGTAACGCCAATAGGGACTTTCCAT
TGACGTCAATGGGTGGAGTATTTACGGTAAACTGCCCACTTGGCAGTACATCAAGTGTATCA
TATGCCAAGTACGCCCCCTATTGACGTCAATGACGGTAAATGGCCCGCCTGGCATTATGCCC
AGTACATGACCTTATGGGACTTTCCTACTTGGCAGTACATCTACGTATTAGTCATCGCTATT
ACCATGGTGATGCGGTTTTGGCAGTACATCAATGGGCGTGGATAGCGGTTTGACTCACGGGG
ATTTCCAAGTCTCCACCCCATTGACGTCAATGGGAGTTTGTTTTGGCACCAAAATCAACGGG
ACTTTCCAAAATGTCGTAACAACTCCGCCCCATTGACGCAAATGGGCGGTAGGCGTGTACGG
TGGGAGGTCTATATAAGCAGAGCTCGTTTAGTGAACCGGGGTCTCTCTGGTTAGACCAGATC
TGAGCCTGGGAGCTCTCTGGCTAACTAGGGAACCCACTGCTTAAGCCTCAATAAAGCTTGCC
TTGAGTGCTTCAAGTAGTGTGTGCCCGTCTGTTGTGTGACTCTGGTAACTAGAGATCCCTCA
GACCCTTTTAGTCAGTGTGGAAAATCTCTAGCAGTGGCGCCCGAACAGGGACCTGAAAGCGA
AAGGGAAACCAGAGCTCTCTCGACGCAGGACTCGGCTTGCTGAAGCGCGCACGGCAAGAGGC
GAGGGGCGGCGACTGGTGAGTACGCCAAAAATTTTGACTAGCGGAGGCTAGAAGGAGAGAGA
TGGGTGCGAGAGCGTCAGTATTAAGCGGGGGAGAATTAGATCGCGATGGGAAAAAATTCGGT
TAAGGCCAGGGGGAAAGAAAAAATATAAATTAAAACATATAGTATGGGCAAGCAGGGAGCTA
GAACGATTCGCAGTTAATCCTGGCCTGTTAGAAACATCAGAAGGCTGTAGACAAATACTGGG
ACAGCTACAACCATCCCTTCAGACAGGATCAGAAGAACTTAGATCATTATATAATACAGTAG
CAACCCTCTATTGTGTGCATCAAAGGATAGAGATAAAAGACACCAAGGAAGCTTTAGACAAG
ATAGAGGAAGAGCAAAACAAAAGTAAGACCACCGCACAGCAAGCGGCCGCTGATCTTCAGAC
CTGGAGGAGGAGATATGAGGGACAATTGGAGAAGTGAATTATATAAATATAAAGTAGTAAAA
ATTGAACCATTAGGAGTAGCACCCACCAAGGCAAAGAGAAGAGTGGTGCAGAGAGAAAAAAG
AGCAGTGGGAATAGGAGCTTTGTTCCTTGGGTTCTTGGGAGCAGCAGGAAGCACTATGGGCG
CAGCCTCAATGACGCTGACGGTACAGGCCAGACAATTATTGTCTGGTATAGTGCAGCAGCAG
AACAATTTGCTGAGGGCTATTGAGGCGCAACAGCATCTGTTGCAACTCACAGTCTGGGGCAT
CAAGCAGCTCCAGGCAAGAATCCTGGCTGTGGAAAGATACCTAAAGGATCAACAGCTCCTGG
GGATTTGGGGTTGCTCTGGAAAACTCATTTGCACCACTGCTGTGCCTTGGAATGCTAGTTGG
AGTAATAAATCTCTGGAACAGATTTGGAATCACACGACCTGGATGGAGTGGGACAGAGAAAT
TAACAATTACACAAGCTTAATACACTCCTTAATTGAAGAATCGCAAAACCAGCAAGAAAAGA
ATGAACAAGAATTATTGGAATTAGATAAATGGGCAAGTTTGTGGAATTGGTTTAACATAACA
AATTGGCTGTGGTATATAAAATTATTCATAATGATAGTAGGAGGCTTGGTAGGTTTAAGAAT
AGTTTTTGCTGTACTTTCTATAGTGAATAGAGTTAGGCAGGGATATTCACCATTATCGTTTC
AGACCCACCTCCCAACCCCGAGGGGACCCGACAGGCCCGAAGGAATAGAAGAAGAAGGTGGA
GAGAGAGACAGAGACAGATCCATTCGATTAGTGAACGGATCTCGACGGTATCGGTTAACTTT
TAAAAGAAAAGGGGGGATTGGGGGGTACAGTGCAGGGGAAAGAATAGTAGACATAATAGCAA
CAGACATACAAACTAAAGAATTACAAAAACAAATTACAAAAATTCAAAATTTTATCGATCAC
GAGACTAGCCTCGACGGTATCGATAAGCTTGATGATATCCCATAGAGCCCACCGCATCCCCA
GCATGCCTGCTATTGTCTTCCCAATCCTCCCCCTTGCTGTCCTGCCCCACCCCACCCCCCAG
AATAGAATGACACCTACTCAGACAATGCGATGCAATTTCCTCATTTTATTAGGAAAGGGCAG
TGGGAGTGGCACCTTCCAGGGTCAAGGAAGGCACGGGGGAGGGGCAAACAACAGATGGCTGG
CAACTAGAAGGCACAGCGTACGGTCATGTAGTGTTTCCTACTTTATGGAGTGATGTAGTGTT
TCCTACTTTATGGAACCGGTTGTAGTGTTTCCTACTTTATGGAATCGTGTAGTGTTTCCTAC
TTTATGGAATCGGTCGAGGTTTAAACTCATGCCACATCGTCCTCCAGGCTGACCATCTGTCT
TGAGGGGTAGGTATAGCCATCCTGGCTTCGGCAAATGTGGAGCTCGTCCTCTGTGGTCTTCT
GGTAGACTGGGTTGTCAAAGTTTATGCTGTTGATGTTCTTCAGCCGCCAGTTCCTCCACAGC
AGGACGGCCCCAAGGACAAGGAGGGCAACCAGTGCAATAGGGAAGAAGATGGACAGGAACCT
CATACCATGTGGCTGCTCCTCATTCCCTCTGCCAGCCATGTCACCCTGGACTTGGTGGGACA
CTGTCACTGACGCCACTGTGCTGAGCCCTGGGGTGTCCACAGGCTGCCTAGGAGTACTGGGA
GCTGAGAGATCCTCACTGTGCTTCGGTGGCCTGGTAGCTGATGCGGTGACCACAGGCCGGAC
GGCGGATGTCCCCTGGGTGGTCAGTACAGTGTCGACTTCTGTGAGGCAGCTCCGCATGTCCT
TGGCCAGCAGCATGCCATCAGGGCAGGCGCAGGTGAATTTGGGCGAGTGGGGACCGATCTGT
GGGGCGGGCAGGCACAGGTACTGGCAACCACCATTGGGGAGGAGGGCTGTTGTCTCACACCA
GTTCACCCCTCTAGGCTGTGTGACCTTGTGGAACAGGACAATGTCCTCCGGGGACAAGAGGT
TTTCAGCCACCAAATTCACATCTGAACCCGTGAGTCGATTGGCACTGAAAATGGCTTCGTTT
ATGACATCTGTCCAATACACTTTGTCCTCATAGATGGCCAAGGAGAAGGGGTGGGCCAGCCG
GTTCTCATCCTCCAAAATGGTTTTCCGATTGCCCCCATTGACATCGATGCTGGAGATAGAGT
GGAGTTTGGAATCAACCCAATAGAGACGGCCACTGGAAAGATCTAGTGTGATGCCATTTGGC
CACTGGATGTTTTCGGTCACCAGTGAGTGGATGTCCACACCATTCAAACCCCCTTTCTTGAT
CTTGGCGGGTGTTCCCCAATCTGTCCAGTACATGAAGCCATGCACAGGGTCCACTACGATGG
CTCTGGGTCTGGACCCTGCCTCTTGGAACAGTGTCCTCCTCTTTACGCCCTTGGTGTCAGCC
ACAGATACGCTGCCTGGGACTGAATCTGTCCAGTAGATGTTGCGGTGGATCCAGTCTACCGC
CAGCCCGTCAGGGGCATGCAGGTCCTCACTGATGATGGTGTCGTAGGACAAGTTAGGGGCCT
GGTCCATCAGGGCGCTGTAGATCTTTTTTTGGGACAGGTCGGACCAGTAGATTCTATTGTTG
GTCACCTCCGTGTCGAGAGCCACCACATTCTTCAGGTTGGGGAGCAGACTGGTGTACTCGCT
GCGGTCCAGGGTCATCTTCCGGACCTCGTGGCGGTTGGTGAAGAGCAGATAGCCTATGGAGC
CCACAGCCTTGCAGACCCTGGTGTGTGGGTCCATGTGGAAGCCGGCCTGGCACTCACACTTG
TAGCTGCCTTCCAGGTTCACACAGAGCTGGCTGCAGGTGTCTGGCTCCTGACACTCGTCAAT
ATCTTCACACCTGTGGAGGTCCACCAACCGGAAGCCGCTGGGACACAGGCACTCAGAGCCAA
TCTTGAGGTCCTTGCAGATGTGGGAACAGCCACCATTGTTGTCCAAACACTCGTTGGTCTTG
CACTCCTTGATGGGCTCATCCGACCAGTCCTGGCAGTCGCGGGCGGAGTCGCACACCTTGTC
CAAGCTGATGCACTCCCCACTGTGACACTTGAACTTGTTGGGGCCATCACACTGTGTCACAT
TGACGCAGCCGAGCTCGTCGCTCATGTCCTTGCAGTCATGTTCACGGTCACACTGGCGGCTA
CCGTGAATGCAGGAGCCATCTGCACACTGGAATTCATCAGGTCGGCAGGTGGCCACCGCGCA
GTGCTCCTCATCTGACTTGTCCTTGCAGTCTGCCTCGCCGTCACAGACCCAGCTGCGATGGA
TACACTCACTGCTACCACAGTGGAACTCCAGGGAGGAGCAGGGGCTGCTAACGCCTTTGGAG
GCCGTGTCTCGGCCCTGGCAGTTCTGTGGCCACTCATCGGAGCCGTCAACACAGTCGACATC
CCCGTCGCAGGCCCAAAGACTGGGGATGCATATGGATGAGTTGCAGCGGAAGTGGGCGGGGC
CACAAGTGGTGGCCTGGCAGTGGGCCTCATCAGAGCCATCTAGGCAATCTCGGTCTCCATCA
CACACAAACTGCGGGGAGATGCACTTGCCATCCTGGCATCGGAAGTCATCCTGGGAGCACGT
CTTGGGGGGACAGCCTTGTTCGTCTGAGTCATTTTCACAGTCTACCTGTCCATCACATCTCC
AGGAGTCAGGAATGCATCGGCTGACACGGCCTCCACAGCTGAATTGATTGGACTGACAGGTG
ACAGACATGCATGTCTCTGGGGACTCATCGGAGCCATCCGGGCACTCGGGGCTGCCATCGCA
CACCCACTTGCTAGCGATGCATTTTCCGTCTCTACACTGGAACTCGTTCCTGCTGCATGAGT
CTTCTGCTGCAACTCCGGCAGCAGCCAGGAGCAGGGCGATGACCCAGCGACGCATCAGATCC
GCGGTGCTCATGGTGGCACGCGTTCCGGTGGATCCACTAGCCAGGAGCTTGTGGATCTGTGT
GACGGCTTCTCCTGGTGAAGGGGCTTTTATACCCCCTCCTTCCAACCCAGGCTGCTGATCCC
TGCCAAGCTGACTCCAAACCTGCTGATTCTGATTATTGACTTAGTCAACAAAAGGAGAATAA
GTAACCTACACAAATATGAACCTTGCCTAGGGAGATTAGAGTATCGGAACACTCGCTCTACG
AAATGTGCAGACAGACGGGGATCCTCTAGAGCTACCTGCTGATCGCCCGGCCCCTGTTCAAA
CATGTCCTAATACTCTGTCTCTGCAAGGGTCATCAGTAGTTTTCCATCTTACTCAACATCCT
CCCAGTGCCCTCGACCTGCAGCCCAAGCTCCTAAGCTTATCGATACCGTCCGGGGCGGGGCG
AACGCGCGCTGGGCAAAGGTCACCTGCCCCTCGCGATTGCGGCAATCGCGGCCTCGAATCAA
TATTCGCGAGGTTAATAATTACCAGCGCGCGCTGGGCAAACGTCACCTGCCCCTCGCGGATT
ATTTATTTGGCCCGCGCTGGTAATTATTAACTCGCGTGCTCGACAATCAACCTCTGGATTAC
AAAATTTGTGAAAGATTGACTGGTATTCTTAACTATGTTGCTCCTTTTACGCTATGTGGATA
CGCTGCTTTAATGCCTTTGTATCATGCTATTGCTTCCCGTATGGCTTTCATTTTCTCCTCCT
TGTATAAATCCTGGTTGCTGTCTCTTTATGAGGAGTTGTGGCCCGTTGTCAGGCAACGTGGC
GTGGTGTGCACTGTGTTTGCTGACGCAACCCCCACTGGTTGGGGCATTGCCACCACCTGTCA
GCTCCTTTCCGGGACTTTCGCTTTCCCCCTCCCTATTGCCACGGCGGAACTCATCGCCGCCT
GCCTTGCCCGCTGCTGGACAGGGGCTCGGCTGTTGGGCACTGACAATTCCGTGGTGTTGTCG
GGGAAATCATCGTCCTTTCCTTGGCTGCTCGCCTGTGTTGCCACCTGGATTCTGCGCGGGAC
GTCCTTCTGCTACGTCCCTTCGGCCCTCAATCCAGCGGACCTTCCTTCCCGCGGCCTGCTGC
CGGCTCTGCGGCCTCTTCCGCGTCTTCGCCTTCGCCCTCAGACGAGTCGGATCTCCCTTTGG
GCCGCCTCCCCGCCTGGAATTCGAGCTCGGTACCTTTAAGACCAATGACTTACAAGGCAGCT
GTAGATCTTAGCCACTTTTTAAAAGAAAAGGGGGGACTGGAAGGGCTAATTCACTCCCAACG
AAGACAAGATCTGCTTTTTGCTTGTACTGGGTCTCTCTGGTTAGACCAGATCTGAGCCTGGG
AGCTCTCTGGCTAACTAGGGAACCCACTGCTTAAGCCTCAATAAAGCTTGCCTTGAGTGCTT
CAAGTAGTGTGTGCCCGTCTGTTGTGTGACTCTGGTAACTAGAGATCCCTCAGACCCTTTTA
GTCAGTGTGGAAAATCTCTAGCAGTAGTAGTTCATGTCATCTTATTATTCAGTATTTATAAC
TTGCAAAGAAATGAATATCAGAGAGTGAGAGGAACTTGTTTATTGCAGCTTATAATGGTTAC
AAATAAAGCAATAGCATCACAAATTTCACAAATAAAGCATTTTTTTCACTGCATTCTAGTTG
TGGTTTGTCCAAACTCATCAATGTATCTTATCATGTCTGGCTCTAGCTATCCCGCCCCTAAC
TCCGCCCAGTTCCGCCCATTCTCCGCCCCATGGCTGACTAATTTTTTTTATTTATGCAGAGG
CCGAGGCCGCCTCGGCCTCTGAGCTATTCCAGAAGTAGTGAGGAGGCTTTTTTGGAGGCCTA
GGCTTTTGCGTCGAGACGTACCCAATTCGCCCTATAGTGAGTCGTATTACGCGCGCTCACTG
GCCGTCGTTTTACAACGTCGTGACTGGGAAAACCCTGGCGTTACCCAACTTAATCGCCTTGC
AGCACATCCCCCTTTCGCCAGCTGGCGTAATAGCGAAGAGGCCCGCACCGATCGCCCTTCCC
AACAGTTGCGCAGCCTGAATGGCGAATGGCGCGACGCGCCCTGTAGCGGCGCATTAAGCGCG
GCGGGTGTGGTGGTTACGCGCAGCGTGACCGCTACACTTGCCAGCGCCCTAGCGCCCGCTCC
TTTCGCTTTCTTCCCTTCCTTTCTCGCCACGTTCGCCGGCTTTCCCCGTCAAGCTCTAAATC
GGGGGCTCCCTTTAGGGTTCCGATTTAGTGCTTTACGGCACCTCGACCCCAAAAAACTTGAT
TAGGGTGATGGTTCACGTAGTGGGCCATCGCCCTGATAGACGGTTTTTCGCCCTTTGACGTT
GGAGTCCACGTTCTTTAATAGTGGACTCTTGTTCCAAACTGGAACAACACTCAACCCTATCT
CGGTCTATTCTTTTGATTTATAAGGGATTTTGCCGATTTCGGCCTATTGGTTAAAAAATGAG
CTGATTTAACAAAAATTTAACGCGAATTTTAACAAAATATTAACGTTTACAATTTCC

### Variants, Derivatives, Analogues and Fragments

In addition to the specific polypeptides and polynucleotides mentioned herein, the invention also encompasses variants, derivatives and fragments thereof.

In the context of the invention, a “variant” of any given sequence is a sequence in which the specific sequence of residues (whether amino acid or nucleic acid residues) has been modified in such a manner that the polypeptide or polynucleotide in question retains at least one or all of its endogenous functions. A variant sequence can be obtained by addition, deletion, substitution, modification, replacement and/or variation of at least one residue present in the naturally occurring polypeptide or polynucleotide.

The term “derivative” as used herein in relation to proteins or polypeptides of the invention includes any substitution of, variation of, modification of, replacement of, deletion of and/or addition of one (or more) amino acid residues from or to the sequence, providing that the resultant protein or polypeptide retains at least one or all of its endogenous functions.

Typically, amino acid substitutions may be made, for example from 1, 2 or 3, to 10 or 20 substitutions, provided that the modified sequence retains the required activity or ability. Amino acid substitutions may include the use of non-naturally occurring analogues.

Polypeptides used in the invention may also have deletions, insertions or substitutions of amino acid residues which produce a silent change and result in a functionally equivalent polypeptide. Deliberate amino acid substitutions may be made on the basis of similarity in polarity, charge, solubility, hydrophobicity, hydrophilicity and/or the amphipathic nature of the residues as long as the endogenous function is retained. For example, negatively charged amino acids include aspartic acid and glutamic acid; positively charged amino acids include lysine and arginine; and amino acids with uncharged polar head groups having similar hydrophilicity values include asparagine, glutamine, serine, threonine and tyrosine.

Conservative substitutions may be made, for example according to the table below. Amino acids in the same block in the second column and in the same line in the third column may be substituted for each other:

| | | |
|---|---|---|
| ALIPHATIC | Non-polar | G A P |
| | | I L V |
| | Polar - uncharged | C S T M |
| | | N Q |
| | Polar - charged | D E |
| | | K R H |
| AROMATIC | | F W Y |

The effect of additions, deletions, substitutions, modifications, replacements and/or variations may be predicted using any suitable prediction tool, for example SIFT (Vaser et al. (2016) Nature Protocols 11: 1-9), PolyPhen-2 (Adzhubei et al. (2013) Current Protocols in Human Genetics 76: 7-20), CADD (Rentzsch et al. (2021) Genome Medicine 13: 1-12), REVEL (loannidis et al. (2016) The American Journal of Human Genetics 99: 877-885), MetaLR (Dong et al. (2015) Human Molecular Genetics 24: 2125-2137) and/or MutationAssessor (Reva et al. (2011) Nucleic Acids Research 39: e118-e118) or based on clinical data, for example ClinVar (Landrum et al. (2016) Nucleic Acids Research 44: D862-D868). Suitable additions, deletions, substitutions, modifications, replacements and/or variations may be considered tolerated, benign and/or likely benign.

Typically, a variant may have a certain identity with the wild type amino acid sequence or the wild type nucleotide sequence.

In the present context, a variant sequence is taken to include an amino acid sequence which may be at least 50%, at least 55%, at least 60%, at least 65%, at least 70%, at least 75%, at least 80%, at least 85% or at least 90% identical, suitably at least 95%, at least 96%, at least 97%, at least 98% or at least 99% identical to the subject sequence. Although a variant can also be considered in terms of similarity (i.e. amino acid residues having similar chemical properties/functions), in the context of the present invention it is preferred to express in terms of sequence identity.

In the present context, a variant sequence is taken to include a nucleotide sequence which may be at least 50%, at least 55%, at least 60%, at least 65%, at least 70%, at least 75%, at least 80%, at least 85% or at least 90% identical, suitably at least 95%, at least 96%, at least 97%, at least 98% or at least 99% identical to the subject sequence. Although a variant can also be considered in terms of similarity, in the context of the present invention it is preferred to express it in terms of sequence identity.

Suitably, reference to a sequence which has a percent identity to any one of the SEQ ID NOs described herein refers to a sequence that has the stated percent identity over the entire length of the SEQ ID NO referred to.

Sequence identity comparisons can be conducted by eye, or more usually, with the aid of readily available sequence comparison programs. These commercially available computer programs can calculate percent identity between two or more sequences.

Percent identity may be calculated over contiguous sequences, i.e. one sequence is aligned with the other sequence and each amino acid or nucleotide in one sequence is directly compared with the corresponding amino acid or nucleotide in the other sequence, one residue at a time. This is called an “ungapped” alignment. Typically, such ungapped alignments are performed only over a relatively short number of residues.

Although this is a very simple and consistent method, it fails to take into consideration that, for example, in an otherwise identical pair of sequences, one insertion or deletion in the amino acid or nucleotide sequence may cause the following residues or codons to be put out of alignment, thus potentially resulting in a large reduction in percent identity when a global alignment is performed. Consequently, most sequence comparison methods are designed to produce optimal alignments that take into consideration possible insertions and deletions without penalising unduly the overall identity score. This is achieved by inserting “gaps” in the sequence alignment to try to maximise local identity.

However, these more complex methods assign “gap penalties” to each gap that occurs in the alignment so that, for the same number of identical amino acids or nucleotides, a sequence alignment with as few gaps as possible, reflecting higher relatedness between the two compared sequences, will achieve a higher score than one with many gaps. “Affine gap costs” are typically used that charge a relatively high cost for the existence of a gap and a smaller penalty for each subsequent residue in the gap. This is the most commonly used gap scoring system. High gap penalties will produce optimised alignments with fewer gaps. Most alignment programs allow the gap penalties to be modified. However, it is preferred to use the default values when using such software for sequence comparisons. For example when using the GCG Wisconsin Bestfit package the default gap penalty for amino acid sequences is −12 for a gap and −4 for each extension.

Calculation of maximum percent identity therefore firstly requires the production of an optimal alignment, taking into consideration gap penalties. A suitable computer program for carrying out such an alignment is the GCG Wisconsin Bestfit package (Devereux et al. (1984) Nucleic Acids Research 12: 387-395). Examples of other software that can perform sequence comparisons include, but are not limited to, the BLAST package (Altschul, et al. (1990) Journal of Molecular Biology 215: 403-410), BLAST 2 (Tatusova et al. (1999) FEMS Microbiology Letters 174: 247-250), FASTA (Pearson et al. (1988) PNAS 85: 2444-2448), EMBOSS Needle (Madeira et al. (2019) Nucleic Acids Research 47: W636-W641) and the GENEWORKS suite of comparison tools. For some applications, it is preferred to use EMBOSS Needle.

Although the final percent identity can be measured, the alignment process itself is typically not based on an all-or-nothing pair comparison. Instead, a scaled similarity score matrix is generally used that assigns scores to each pairwise comparison based on chemical similarity or evolutionary distance. An example of such a matrix commonly used is the BLOSUM62 matrix.

Once the software has produced an optimal alignment, it is possible to calculate percent sequence identity. The software typically does this as part of the sequence comparison and generates a numerical result. The percent sequence identity may be calculated as the number of identical residues as a percentage of the total residues in the SEQ ID NO referred to.

“Fragments” are also variants and the term typically refers to a selected region of the polypeptide or polynucleotide that is of interest either functionally or, for example, in an assay. “Fragment” thus refers to an amino acid or nucleic acid sequence that is a portion of a full-length polypeptide or polynucleotide.

Such variants, derivatives and fragments may be prepared using standard recombinant DNA techniques, such as site-directed mutagenesis. Where insertions are to be made, synthetic DNA encoding the insertion together with 5′ and 3′ flanking regions corresponding to the naturally-occurring sequence either side of the insertion site may be made. The flanking regions will contain convenient restriction sites corresponding to sites in the naturally-occurring sequence so that the sequence may be cut with the appropriate enzyme(s) and the synthetic DNA ligated into the cut. The DNA is then expressed in accordance with the invention to make the encoded polypeptide. These methods are only illustrative of the numerous standard techniques known in the art for manipulation of DNA sequences and other known techniques may also be used.

### Method of Production

In one aspect, the present invention provides a method of producing the lentiviral vector of the present invention.

Suitable methods to produce lentiviral vectors will be well known to the skilled person (see, for example, Merten et al. (2016) Molecular Therapy-Methods & Clinical Development 3: 16017).

The method of production may comprise: (a) introducing a transfer vector and one or more helper vectors into a host cell; (b) culturing the host cell under conditions suitable to produce lentiviral vectors according to the present invention; and (c) obtaining the lentiviral vectors from the host cell.

As used herein, a “transfer vector” may encode the lentiviral genome of the present invention. Suitably, the transfer vector used to produce the lentiviral genome within a host cell/packaging cell will have sufficient lentiviral genetic information to allow packaging of an RNA genome, in the presence of packaging components (e.g. gag-pol, rev, env), into a viral particle which is capable of infecting a target cell, but is incapable of independent replication to produce infectious viral particles within the final target cell.

The transfer vector used to produce the viral genome within a host cell/packaging cell may include transcriptional regulatory control sequences operably linked to the lentiviral genome to direct transcription of the genome in a host cell/packaging cell. These regulatory sequences may be the natural sequences associated with the transcribed viral sequence (i.e. the 5′ U3 region), or they may be a heterologous promoter, such as another viral promoter (e.g. the CMV promoter). The transfer vector may be a plasmid.

As used herein, a “helper vector” may encode one or more packaging components (e.g. gag-pol, rev, env). The nucleotide sequence encoding the packaging component(s) may be operably linked to a promoter (e.g. a CMV promoter or a RSV promoter) and/or a polyadenylation signal. The term “helper vector” may include “packaging vectors” (e.g. encoding gag-pol or rev) and “envelope vectors” (e.g. encoding an env gene, such as VSV-g). The helper vectors, packaging vectors and/or envelope vectors may be plasmids.

The transfer vector and one or more helper vectors may be introduced into the host cell by any suitable technique known in the art, such as transfection, transduction and/or transformation. Suitably, the helper vectors may be transiently transfected or transduced into the host cell or may be stably maintained (e.g. stably integrated into the cell genome) within the host cell. Alternatively, a combination of transient transfection or transduction and stable maintenance may be used to introduce the helper vectors into the host cell.

Suitably, the transfer vector and/or the helper vectors may be plasmids and introduced by transfection. Suitably, a four plasmid system may be used consisting of a transfer plasmid and three helper plasmids. The three helper plasmids may consist of: a first helper plasmid encoding a gag-pol gene; a second helper plasmid encoding a rev gene; and a third helper plasmid encoding an env gene. Alternatively, a three plasmid system may be used which consists of a transfer plasmid, one helper plasmid encoding a gag-pol gene and a rev gene; and one helper plasmid encoding an env gene. Alternatively, a two plasmid system may be used in which all helper functions (e.g. gag-pol, rev and env) are encoded by one helper plasmid.

Any suitable host cell may be used to produce the lentiviral vector. Suitable host cells include producer cells and packaging cells, such as those described below (e.g. HEK 293 or derivatives thereof). Suitable conditions for culturing the host cell will be well known to the skilled person. For example, the host cells may be incubated in chemically defined medium for from about 1 day to about 5 days (e.g. about 48 hours, about 72 hours or about 96 hours).

The lentiviral vector may be obtained using any suitable methods known in the art. For example the culture supernatant may be harvested and lentiviral vector subsequently purified from the culture supernatant (e.g. by centrifugation, membrane filtration and/or chromatography). The method of production may further comprise any other suitable process steps, for example DNA reduction, concentration, formulation and/or sterilisation.

The method of producing the lentiviral vector may use a producer or packaging cell, wherein the cell is modified to decrease expression of low density lipoprotein receptor (LDLR) on the surface of the cell. The cell may be, for example, modified (e.g. genetically engineered) to overexpress pro-protein convertase subtilisin/Kexin type 9 (PCSK9). In some embodiments, the cell comprises a heterologous polynucleotide comprising a nucleotide sequence encoding pro-protein convertase subtilisin/Kexin type 9 (PCSK9).

Suitably, the lentiviral vector of the invention may be produced in a cell that is modified to decrease expression of LDLR on the surface of the cell, wherein the nucleotide sequence encoding LDLR is in a sense orientation in the lentiviral vector. Use of said cell may increase titer and infectivity of the lentiviral vector. Use of said cell may rescue lentiviral vector production.

A method of producing the lentiviral vector may, for example, comprise the steps: (a) introducing a transfer vector and optionally one or more helper vector into a cell; (b) introducing a vector comprising a nucleotide sequence encoding pro-protein convertase subtilisin/Kexin type 9 (PCSK9) into the cell; (c) culturing the cell under conditions suitable for the production of the lentiviral vector. In some embodiments, the steps (a), (b) and (c) are carried our consecutively in the order listed. In some embodiments, steps (a) and (b) are carried out at the same time. In some embodiments, step (b) is carried out before step (a), optionally to integrate the nucleotide sequence encoding PCSK9 into the genome of the cell.

An example PCSK9 amino acid sequence is:

(SEQ ID NO: 40)
MGTVSSRRSWWPLPLLLLLLLLLGPAGARAQEDEDGDYEELVLALRSEEDGLAEAPEHGTTATFHRCA
KDPWRLPGTYVVVLKEETHLSQSERTARRLQAQAARRGYLTKILHVFHGLLPGFLVKMSGDLLELALK
LPHVDYIEEDSSVFAQSIPWNLERITPPRYRADEYQPPDGGSLVEVYLLDTSIQSDHREIEGRVMVTD
FENVPEEDGTRFHRQASKCDSHGTHLAGVVSGRDAGVAKGASMRSLRVLNCQGKGTVSGTLIGLEFIR
KSQLVQPVGPLVVLLPLAGGYSRVLNAACQRLARAGVVLVTAAGNFRDDACLYSPASAPEVITVGATN
AQDQPVTLGTLGTNFGRCVDLFAPGEDIIGASSDCSTCFVSQSGTSQAAAHVAGIAAMMLSAEPELTL
AELRQRLIHFSAKDVINEAWFPEDQRVLTPNLVAALPPSTHGAGWQLFCRTVWSAHSGPTRMATAVAR
CAPDEELLSCSSFSRSGKRRGERMEAQGGKLVCRAHNAFGGEGVYAIARCCLLPQANCSVHTAPPAEA
SMGTRVHCHQQGHVLTGCSSHWEVEDLGTHKPPVLRPRGQPNQCVGHREASIHASCCHAPGLECKVKE
HGIPAPQEQVTVACEEGWTLTGCSALPGTSHVLGAYAVDNTCVVRSRDVSTTGSTSEGAVTAVAICCR
SRHLAQASQELQ

In some embodiments, the PCSK9 comprises or consists of an amino acid sequence that has at least 75%, 80%, 85%, 90%, 95%, 96%, 97%, 98% or 99% sequence identity to SEQ ID NO: 40, or a fragment thereof. In some embodiments, the PCSK9 comprises or consists of the amino acid sequence of SEQ ID NO: 40, or a fragment thereof.

In preferred embodiments, the PCSK9 comprises the mutation S127R, wherein the amino acids are numbered with reference to SEQ ID NO: 40. The terms “corresponding to”, “reference to” and “relative to” when used in the context of the numbering of a given amino acid or polynucleotide sequence may refer to the numbering of the residues of a specified reference sequence when the given amino acid or polynucleotide sequence is compared to the reference sequence. In other words, the residue number or residue position of a given polymer is designated with respect to the reference sequence rather than by the actual numerical position of the residue within the given amino acid or polynucleotide sequence. For example, a given amino acid sequence, such as that of a PCSK9, can be aligned to a reference sequence by introducing gaps to optimise residue matches between the two sequences. In these cases, although the gaps are present, the numbering of the residue in the given amino acid or polynucleotide sequence is made with respect to the reference sequence to which it has been aligned.

An example PCSK9 S127R amino acid sequence is:

(SEQ ID NO: 41)
MGTVSSRRSWWPLPLLLLLLLLLGPAGARAQEDEDGDYEELVLALRSEEDGLAEAPEHGTTATFHRCA
KDPWRLPGTYVVVLKEETHLSQSERTARRLQAQAARRGYLTKILHVFHGLLPGFLVKMRGDLLELALK
LPHVDYIEEDSSVFAQSIPWNLERITPPRYRADEYQPPDGGSLVEVYLLDTSIQSDHREIEGRVMVTD
FENVPEEDGTRFHRQASKCDSHGTHLAGVVSGRDAGVAKGASMRSLRVLNCQGKGTVSGTLIGLEFIR
KSQLVQPVGPLVVLLPLAGGYSRVLNAACQRLARAGVVLVTAAGNFRDDACLYSPASAPEVITVGATN
AQDQPVTLGTLGTNFGRCVDLFAPGEDIIGASSDCSTCFVSQSGTSQAAAHVAGIAAMMLSAEPELTL
AELRQRLIHFSAKDVINEAWFPEDQRVLTPNLVAALPPSTHGAGWQLFCRTVWSAHSGPTRMATAVAR
CAPDEELLSCSSFSRSGKRRGERMEAQGGKLVCRAHNAFGGEGVYAIARCCLLPQANCSVHTAPPAEA
SMGTRVHCHQQGHVLTGCSSHWEVEDLGTHKPPVLRPRGQPNQCVGHREASIHASCCHAPGLECKVKE
HGIPAPQEQVTVACEEGWTLTGCSALPGTSHVLGAYAVDNTCVVRSRDVSTTGSTSEGAVTAVAICCR
SRHLAQASQELQ

In some embodiments, the PCSK9 comprises or consists of an amino acid sequence that has at least 75%, 80%, 85%, 90%, 95%, 96%, 97%, 98% or 99% sequence identity to SEQ ID NO: 41, or a fragment thereof. Preferably, the PCSK9 comprises the mutation S127R, wherein the amino acids are numbered with reference to SEQ ID NO: 40. In some embodiments, the PCSK9 comprises or consists of the amino acid sequence of SEQ ID NO: 41, or a fragment thereof.

Example nucleotide sequences encoding PCSK9 include:

(SEQ ID NO: 42; codon-optimised PCSK9)
ATGGGCACCGTGTCCAGCAGACGGTCTTGGTGGCCTCTGCCTCTGCTGTTGCTGCTGCTTCTTCTGCT
TGGACCTGCTGGCGCTAGAGCCCAAGAGGATGAGGATGGCGACTACGAGGAACTGGTGCTGGCCCTGA
GAAGCGAGGAAGATGGACTGGCCGAAGCTCCTGAGCACGGCACCACAGCCACCTTCCACAGATGCGCC
AAAGATCCTTGGAGACTGCCCGGCACATACGTGGTGGTGCTGAAAGAGGAAACCCACCTGAGCCAGAG
CGAGAGAACAGCCAGAAGGCTGCAGGCTCAGGCCGCCAGAAGAGGCTACCTGACCAAGATCCTGCACG
TGTTCCACGGCCTGCTGCCTGGCTTTCTGGTCAAGATGTCTGGCGATCTGCTGGAACTGGCTCTGAAG
CTGCCTCACGTGGACTACATCGAAGAGGACAGCAGCGTGTTCGCCCAGAGCATCCCCTGGAACCTGGA
AAGAATCACCCCTCCTAGATACCGGGCCGACGAGTACCAACCTCCTGATGGCGGATCTCTGGTGGAAG
TGTACCTGCTGGATACCAGCATCCAGAGCGACCACCGGGAAATCGAGGGCAGAGTGATGGTCACCGAC
TTCGAGAACGTGCCCGAAGAAGATGGCACCCGGTTTCACAGACAGGCCAGCAAGTGTGATAGCCACGG
AACACATCTGGCCGGCGTGGTGTCTGGAAGAGATGCTGGTGTTGCCAAGGGCGCCAGCATGAGATCTC
TGAGAGTGCTGAACTGCCAAGGCAAGGGCACAGTGTCTGGCACACTGATCGGCCTCGAGTTCATCCGG
AAGTCCCAGCTGGTTCAGCCTGTGGGACCTCTGGTTGTTCTGCTGCCACTGGCTGGCGGCTATAGCAG
GGTTCTGAATGCCGCCTGTCAGAGACTGGCTAGAGCTGGCGTTGTGCTGGTTACAGCCGCCGGAAACT
TCAGAGATGACGCCTGCCTGTACAGCCCTGCCAGTGCTCCTGAAGTGATCACAGTGGGCGCCACAAAC
GCCCAGGATCAGCCTGTTACACTGGGCACCCTGGGCACAAACTTCGGCAGATGCGTGGACCTGTTTGC
CCCTGGCGAGGATATTATCGGCGCCAGCTCCGATTGCAGCACCTGTTTTGTGTCTCAGAGCGGCACCT
CTCAGGCTGCCGCTCATGTTGCTGGAATCGCCGCCATGATGCTGTCTGCCGAGCCTGAACTGACTCTG
GCCGAGCTGAGACAGCGGCTGATCCACTTTAGCGCCAAGGACGTGATCAACGAGGCCTGGTTTCCCGA
GGATCAGAGGGTGCTGACCCCTAATCTGGTGGCTGCTCTGCCACCTTCTACACACGGTGCTGGCTGGC
AGCTGTTCTGCAGGACAGTTTGGAGCGCCCACAGCGGCCCTACAAGAATGGCTACAGCCGTGGCTAGA
TGCGCCCCTGATGAGGAACTGCTGAGCTGCTCCAGCTTCAGCAGAAGCGGCAAGAGAAGAGGCGAGCG
GATGGAAGCCCAAGGCGGAAAACTTGTGTGCAGAGCCCACAATGCCTTTGGCGGAGAAGGCGTGTACG
CCATTGCCAGATGTTGTCTGTTGCCCCAGGCCAACTGCAGCGTGCACACAGCTCCTCCAGCCGAAGCC
TCTATGGGCACCAGAGTGCACTGTCACCAGCAGGGACATGTGCTGACAGGCTGTAGCAGCCACTGGGA
AGTTGAGGACCTGGGAACCCACAAGCCTCCAGTGCTCAGACCTAGAGGCCAGCCTAATCAGTGCGTGG
GACACAGAGAGGCCTCCATCCACGCCTCTTGTTGTCATGCCCCTGGACTGGAATGCAAAGTGAAAGAG
CACGGAATCCCCGCTCCTCAAGAGCAAGTGACCGTGGCCTGTGAAGAAGGCTGGACACTGACCGGATG
TTCTGCCCTGCCTGGCACATCTCATGTGCTGGGAGCCTACGCCGTGGACAATACCTGTGTTGTGCGGA
GCAGAGATGTGTCCACCACCGGCTCTACATCTGAGGGCGCTGTGACAGCTGTGGCCATCTGCTGCAGA
AGCAGACACCTGGCACAGGCCTCTCAAGAGCTGCAGTGA
(SEQ ID NO: 43; codon-optimised PCSK9 S127R)
ATGGGCACCGTGAGCTCCCGGAGAAGCTGGTGGCCTCTGCCACTGTTATTACTGCTGCTGCTGCTGCT
GGGACCAGCAGGAGCAAGGGCCCAGGAGGACGAGGATGGCGACTACGAGGAGCTGGTGCTGGCCCTGC
GCTCCGAGGAGGACGGCCTGGCCGAGGCCCCTGAGCACGGCACCACAGCCACCTTCCACAGGTGCGCA
AAGGACCCCTGGAGGCTGCCAGGCACATACGTGGTGGTGCTGAAGGAGGAGACACACCTGTCCCAGTC
TGAGAGGACCGCAAGGCGCCTGCAGGCACAGGCAGCAAGGAGAGGCTATCTGACCAAGATCCTGCACG
TGTTCCACGGCCTGCTGCCAGGCTTTCTGGTGAAGATGAGGGGCGACCTGCTGGAGCTGGCCCTGAAG
CTGCCACACGTGGATTACATCGAGGAGGACTCTAGCGTGTTTGCCCAGTCTATCCCCTGGAACCTGGA
GAGAATCACCCCCCCTCGGTACAGAGCCGATGAGTATCAGCCACCAGACGGAGGCTCCCTGGTGGAGG
TGTATCTGCTGGATACAAGCATCCAGTCCGACCACCGGGAGATCGAGGGCAGAGTGATGGTGACAGAC
TTCGAGAACGTGCCTGAGGAGGATGGCACCAGGTTTCACCGCCAGGCCTCTAAGTGCGACAGCCACGG
CACCCACCTGGCAGGAGTGGTGAGCGGCCGGGATGCAGGAGTGGCAAAGGGAGCATCTATGCGGAGCC
TGAGAGTGCTGAATTGTCAGGGCAAGGGCACAGTGTCCGGCACCCTGATCGGCCTGGAGTTCATCCGG
AAGTCTCAGCTGGTGCAGCCAGTGGGACCACTGGTGGTGCTGCTGCCACTGGCAGGAGGATACAGCAG
AGTGCTGAACGCAGCATGCCAGAGGCTGGCAAGGGCAGGCGTGGTGCTGGTGACAGCCGCCGGCAACT
TCCGGGACGATGCCTGTCTGTATTCCCCCGCCTCTGCCCCTGAGGTGATCACAGTGGGAGCAACCAAC
GCACAGGACCAGCCTGTGACCCTGGGCACACTGGGCACCAATTTCGGCCGCTGCGTGGATCTGTTTGC
ACCAGGAGAGGACATCATCGGAGCATCCTCTGATTGCAGCACATGTTTCGTGAGCCAGTCCGGCACCT
CCCAGGCTGCCGCCCACGTGGCAGGAATCGCAGCAATGATGCTGAGCGCCGAGCCAGAGCTGACCCTG
GCAGAGCTGAGGCAGCGCCTGATCCACTTCTCCGCCAAGGACGTGATCAACGAGGCCTGGTTTCCTGA
GGATCAGAGGGTGCTGACACCAAATCTGGTGGCCGCCCTGCCTCCAAGCACCCACGGAGCCGGCTGGC
AGCTGTTTTGTAGAACAGTGTGGAGCGCCCACTCCGGACCAACAAGGATGGCAACCGCAGTGGCAAGA
TGCGCACCTGACGAGGAGCTGCTGTCCTGTAGCTCCTTCTCTAGGAGCGGAAAGAGGAGGGGAGAGAG
GATGGAGGCACAGGGAGGAAAGCTGGTGTGCAGGGCACACAACGCCTTTGGCGGAGAGGGCGTGTACG
CAATCGCAAGATGCTGTCTGCTGCCTCAGGCCAATTGTTCTGTGCACACAGCACCACCTGCAGAGGCA
AGCATGGGAACCAGGGTGCACTGCCACCAGCAGGGACACGTGCTGACCGGCTGTTCTAGCCACTGGGA
GGTGGAGGATCTGGGAACACACAAGCCACCCGTGCTGCGGCCAAGAGGACAGCCAAACCAGTGCGTGG
GCCACAGAGAGGCCTCCATCCACGCCTCTTGCTGTCACGCCCCAGGCCTGGAGTGTAAGGTGAAGGAG
CACGGCATCCCCGCACCTCAGGAGCAGGTGACCGTGGCATGCGAGGAGGGATGGACCCTGACAGGATG
TTCTGCCCTGCCAGGCACCAGCCACGTGCTGGGAGCATATGCAGTGGACAATACATGCGTGGTGAGGA
GCCGCGACGTGAGCACCACAGGCTCCACATCTGAGGGAGCAGTGACCGCAGTGGCAATCTGCTGTCGG
TCCAGACACCTGGCCCAGGCCTCTCAGGAGCTGCAGTGA

In some embodiments, the nucleotide sequence encoding PCSK9 comprises or consists of a nucleotide sequence that has at least 75%, 80%, 85%, 90%, 95%, 96%, 97%, 98% or 99% sequence identity to SEQ ID NO: 42, or a fragment thereof. In some embodiments, the nucleotide sequence encoding PCSK9 comprises or consists of the nucleotide sequence of SEQ ID NO: 42, or a fragment thereof.

In some embodiments, the nucleotide sequence encoding PCSK9 comprises or consists of a nucleotide sequence that has at least 75%, 80%, 85%, 90%, 95%, 96%, 97%, 98% or 99% sequence identity to SEQ ID NO: 43, or a fragment thereof. Preferably, the PCSK9 comprises the mutation S127R, wherein the amino acids are numbered with reference to SEQ ID NO: 40. In some embodiments, the nucleotide sequence encoding PCSK9 comprises or consists of the nucleotide sequence of SEQ ID NO: 43, or a fragment thereof.

A fragment and/or variant of PCSK9 may retain PCSK9 activity (e.g. the activity of SEQ ID NO: 40 or 41). For example, a fragment and/or variant of PCSK9 may act as a negative modulator of LDLR and enhance its degradation upon binding. Suitably, a fragment and/or variant of PCSK9 may have the same or similar activity to PCSK9, for example may have at least 40%, at least 50%, at least 60%, at least 70%, at least 80%, at least 90%, at least 95% or 100% of the activity of PCSK9 (e.g. the PCSK9 of SEQ ID NO: 40 or 41). The skilled person will be able to generate fragments and/or variants, for example using conservative substitutions, based on the known structural and functional features of PCSK9.

A “heterologous” polynucleotide (e.g. a heterologous polynucleotide comprising a nucleotide sequence encoding PCSK9) may be a polynucleotide that is not naturally present in the cell, for example has been introduced into the cell by any suitable method, such as transduction or transfection. The heterologous polynucleotide may be, for example, a vector, such as an expression vector. The heterologous polynucleotide may be, for example, a plasmid.

### Vectors, Kits and Systems

In one aspect, the present invention provides a vector encoding the lentiviral genome of the present invention. The vector may be a transfer vector, as described herein. For example, the vector may be a plasmid and/or the lentiviral genome may be operably linked to a promoter (e.g. a viral promoter, such as a CMV promoter).

In one aspect, the present invention provides a kit or system for producing the lentiviral vector of the present invention.

The kit or system may be a lentivirus packaging kit or system or a lentivirus production kit or system. As used herein, a “lentivirus packaging kit or system” may comprise one or more components, and optionally instructions, for packaging the lentiviral vector of the present invention. As used herein, a “lentivirus production kit or system” may comprise one or more components, and optionally instructions, for producing the lentiviral vector of the present invention.

The kit or system may comprise a transfer vector encoding the lentivirus genome of the present invention and optionally one or more helper vectors. The kit or system may further comprise host cells (e.g. packaging cells or producer cells) and/or other reagents (e.g. transfection reagent, culture medium, etc.). The kit or system may further comprise any other suitable components, and optionally instructions for packaging and/or producing the lentiviral vector of the present invention.

### Cells

In one aspect, the present invention provides a cell comprising the lentiviral vector of the present invention. The cell may be an isolated cell. Suitably, the cell is a mammalian cell, for example a human cell. The cell may be an isolated human cell.

Suitably, the cell may be a producer cell. The term “producer cell” may refer to a cell that produces viral particles, for example has been transiently transfected, stably transfected and/or transduced with all the elements necessary to produce the viral particles. Suitable producer cells will be well known to the skilled person and may include HEK293, COS-1, COS-7, CV-1, HeLa, CHO and A549 cell lines. In some embodiments, the producer cell is a HEK293 cell, or a derivative thereof (e.g. a HEK293T cell, a HEK293T Lenti-X, a HEK293T-Rex cell, a HEK293FT cell, a HEK293SF-3F6 cell, a HEK293SF-3F9 cell, a HEK293-EBNA1 cell or a SJ293TS cell).

Suitably, the cell may be a packaging cell. The term “packaging cell” may refer to a cell which contains some or all of the elements necessary for packaging a recombinant virus genome. Typically, such packaging cells contain one or more vectors which are capable of expressing viral structural proteins (e.g. gag-pol, rev, env) and/or one or more genes encoding the viral structural proteins have been integrated into the genome of the packaging cell. Cells comprising only some of the elements required for the production of enveloped viral particles are useful as intermediate reagents in the generation of viral particle producer cell lines, through subsequent steps of transient transfection, transduction or stable integration of each additional required element. These intermediate reagents are encompassed by the term “packaging cell”. Suitable packaging cells will be well known to the skilled person.

Suitably, the cell may be a liver cell, for example a hepatocyte. Suitably, the cell may be an immortalised liver cell, for example an immortalised hepatocyte. Suitable cell lines will be well known to the skilled person, for example HepG2, Hep3B, HBG and HepaRG cell lines. Methods to generate immortalised liver cells (e.g. immortalised hepatocytes) will be well known to the skilled person.

### Pharmaceutical Compositions

In one aspect, the present invention provides a pharmaceutical composition comprising the lentiviral vector or cell of the present invention. In preferred embodiments, the pharmaceutical composition comprises the lentiviral vector of the present invention in the form of a lentiviral particle.

A pharmaceutical composition may be a composition that comprises or consists of a therapeutically effective amount of a pharmaceutically active agent (e.g. the lentiviral vector). A pharmaceutical composition preferably includes a pharmaceutically acceptable carrier, diluent or excipient (including combinations thereof).

By “pharmaceutically acceptable” it is included that the formulation is sterile and pyrogen free. The carrier, diluent, and/or excipient must be “acceptable” in the sense of being compatible with the lentiviral vector and not deleterious to the recipients thereof. Typically, the carriers, diluents and excipients will be saline or infusion media which will be sterile and pyrogen free, however other acceptable carriers, diluents and excipients may be used.

Acceptable carriers, diluents, and excipients for therapeutic use are well known in the pharmaceutical art. The choice of pharmaceutical carrier, excipient or diluent can be selected with regard to the intended route of administration and standard pharmaceutical practice. The pharmaceutical compositions may comprise as (or in addition to) the carrier, excipient or diluent any suitable binder(s), lubricant(s), suspending agent(s), coating agent(s) or solubilising agent(s).

Examples of pharmaceutically acceptable carriers include, for example, water, salt solutions, alcohol, silicone, waxes, petroleum jelly, vegetable oils, polyethylene glycols, propylene glycol, liposomes, sugars, gelatin, lactose, amylose, magnesium stearate, talc, surfactants, silicic acid, viscous paraffin, perfume oil, fatty acid monoglycerides and diglycerides, petroethral fatty acid esters, hydroxymethyl-cellulose, polyvinylpyrrolidone, and the like.

The lentiviral vector, cell, or pharmaceutical composition according to the present invention may be administered in a manner appropriate for treating and/or preventing the diseases described herein. Suitable administration routes will be known to the skilled person.

The quantity and frequency of administration may be determined by the skilled person, for example depending by such factors as the condition of the subject, and the type and severity of the subject's disease. The pharmaceutical composition may be formulated accordingly.

The lentiviral vector, cell or pharmaceutical composition according to the present invention may be administered parenterally, (e.g. intravenous, intra-arterial, intramuscular, intrathecal, subcutaneous), or by infusion techniques. The lentiviral vector, cell or pharmaceutical composition may be administered in the form of a sterile aqueous solution which may contain other substances, for example enough salts or glucose to make the solution isotonic with blood. The aqueous solution may be suitably buffered (preferably to a pH of from 3 to 9). The pharmaceutical composition may be formulated accordingly. The preparation of suitable parenteral formulations under sterile conditions is readily accomplished by standard pharmaceutical techniques well-known to the skilled person.

The lentiviral vector, cell or pharmaceutical composition according to the present invention may be administered systemically, for example by intravenous injection or intraperitoneal injection. In some embodiments, the lentiviral vector, cell or pharmaceutical composition according to the present invention is administered by intravenous injection. The pharmaceutical composition may be formulated accordingly.

The lentiviral vector, cell or pharmaceutical composition according to the present invention may be administered locally, for example by direct injection, intra-arterial injection or intraportal injection. In some embodiments, the lentiviral vector, cell or pharmaceutical composition according to the present invention is administered locally to the liver. In some embodiments, the lentiviral vector, cell or pharmaceutical composition according to the present invention is administered by intrahepatic injection, intrahepatic arterial injection or intraportal injection. The pharmaceutical composition may be formulated accordingly.

The pharmaceutical compositions may comprise lentiviral vectors or cells of the invention in infusion media, for example sterile isotonic solution. The pharmaceutical composition may be enclosed in ampoules, disposable syringes or multiple dose vials made of glass or plastic.

The lentiviral vector, cell or pharmaceutical composition may be administered in a single or in multiple doses. Suitably, the lentiviral vector, cell or pharmaceutical composition may be administered in a single, one off dose. The pharmaceutical composition may be formulated accordingly.

The lentiviral vector, cell or pharmaceutical composition may be administered at varying doses (e.g. measured in Transducing Units (TU) per kg). The physician in any event may determine the actual dosage which will be most suitable for any individual subject and the dosage may, for example, vary with the age, weight and response of the particular subject. The pharmaceutical composition may be formulated accordingly.

The pharmaceutical composition may further comprise one or more other therapeutic agents.

The invention further includes kits comprising the lentiviral vector, cell and/or pharmaceutical composition of the present invention. Preferably said kits are for use in the methods and used as described herein, for example the therapeutic methods as described herein. Preferably said kits comprise instructions for use of the kit components.

Methods for Treating and/or Preventing Disease

In one aspect, the present invention provides the lentiviral vector, cell or pharmaceutical composition according to the present invention for use in therapy.

In one aspect, the present invention provides use of the lentiviral vector, cell or pharmaceutical composition according to the present invention in the manufacture of a medicament.

In one aspect, the present invention provides a method of administering a therapeutically effective amount of the lentiviral vector, cell or pharmaceutical composition according to the present invention to a subject in need thereof.

The lentiviral vector mediated gene therapy described herein may allow for a stable gene transfer even in paediatric patients at the first disease stages by virtue of lentiviral vector genomic integration.

Following administration of the lentiviral vector of the present invention, the lentiviral vector may integrate into the genome of liver cells (e.g. hepatocytes). Subsequently, the lentiviral vector may be maintained in the genome of liver cells (e.g. hepatocytes) as they duplicate.

The integration of the lentiviral vector in the genome of liver cells may be determined by integration site (IS) analysis (e.g. quantitative high-throughput vector IS analysis). Suitable methods are known in the art.

The lentiviral vector, cell or pharmaceutical composition may be administered to any subject in need thereof. The subject may be a mammal (e.g. a human). In preferred embodiments, the lentiviral vector is administered in the form of a lentiviral particle.

In some embodiments, the subject is a juvenile, an adolescent, or a child. The term “juvenile” may refer to an individual that has not yet reached adulthood. The term “adolescent” may refer to an individual during the period from the onset of puberty to adulthood. The term “child” may refer an individual between the stages of birth and puberty.

In some embodiments, the subject is a young child, a toddler or an infant. The term “young child” may refer to a human subject aged from 3 years to 5 years. The term “toddler” may refer to a human subject aged from 1 year to 3 years. The term “infant” may refer to a human subject under the age of 12 months.

In some embodiments, the subject is a paediatric patient. The term “paediatric patient” may refer to a human subject until about 18-21 years of age.

In some embodiments, the subject is a neonatal patient or an infantile patient. The term “neonatal patient” may refer to a human subject who is aged about 4 weeks old or younger. The term “infantile patient” may refer to a human subject who is aged from about 4 weeks to about 1 year.

In other embodiments, the subject is an adult. Human liver is expected to completely renew every 5 years in humans, so integrating vectors are expected to be more persisting compared to mostly episomal vectors (e.g. AAV).

### Familial Hypercholesterolemia (FH)

In one aspect, the invention provides the lentiviral vector of the invention, the cell of the invention or the pharmaceutical composition of the invention for use in therapy.

In one aspect, the invention provides use of the lentiviral vector of the invention, the cell of the invention or the pharmaceutical composition of the invention for the manufacture of a medicament.

In one aspect, the invention provides a method of treatment comprising administering the lentiviral vector of the invention, the cell of the invention or the pharmaceutical composition of the invention to a subject in need thereof.

In one aspect, the invention provides the lentiviral vector of the invention, the cell of the invention or the pharmaceutical composition of the invention for use in treatment or prevention of familial hypercholesterolemia (FH).

In one aspect, the invention provides use of the lentiviral vector of the invention, the cell of the invention or the pharmaceutical composition of the invention for the manufacture of a medicament for treatment or prevention of familial hypercholesterolemia (FH).

In one aspect, the invention provides a method of treatment or prevention of familial hypercholesterolemia (FH) comprising administering the lentiviral vector of the invention, the cell of the invention or the pharmaceutical composition of the invention to a subject in need thereof.

Familial hypercholesterolemia (FH) is an autosomal dominant inherited disorder that is characterised by extremely high LDL cholesterol in the circulation, this condition leads to progressive atherosclerosis. If the homozygous form is left untreated, myocardial infarction usually develops within the first decade of life, leading to death within the third decade. More than 95% of mutations that result in this disorder occur in the gene encoding the low density lipoprotein receptor (LDLR).

Following administration of the lentiviral vector of the present invention to a subject in need thereof, total cholesterol levels and/or LDL cholesterol levels may be reduced and/or normalised, and/or prevented from increasing. Suitably, total cholesterol levels and/or LDL cholesterol levels may be reduced by at least 10%, at least 20%, at least 30%, at least 40%, at least 50%, at least 60%, at least 70%, at least 80% or at least 90%.

### Familial Hypercholesterolemia Associated Conditions, Such as Atherosclerosis.

In one aspect, the invention provides the lentiviral vector of the invention, the cell of the invention or the pharmaceutical composition of the invention for use in treatment or prevention of familial hypercholesterolemia associated conditions, such as atherosclerosis.

In one aspect, the invention provides use of the lentiviral vector of the invention, the cell of the invention or the pharmaceutical composition of the invention for the manufacture of a medicament for treatment or prevention of familial hypercholesterolemia associated conditions, such as atherosclerosis.

In one aspect, the invention provides a method of treatment or prevention of familial hypercholesterolemia associated conditions, such as atherosclerosis, comprising administering the lentiviral vector of the invention, the cell of the invention or the pharmaceutical composition of the invention to a subject in need thereof.

The skilled person will understand that they can combine all features of the invention disclosed herein without departing from the scope of the invention as disclosed.

Preferred features and embodiments of the invention will now be described by way of non-limiting examples.

The practice of the present invention will employ, unless otherwise indicated, conventional techniques of chemistry, biochemistry, molecular biology, microbiology and immunology, which are within the capabilities of a person of ordinary skill in the art. Such techniques are explained in the literature. See, for example, Sambrook, J., Fritsch, E. F. and Maniatis, T. (1989) Molecular Cloning: A Laboratory Manual, 2nd Edition, Cold Spring Harbor Laboratory Press; Ausubel, F. M. et al. (1995 and periodic supplements) Current Protocols in Molecular Biology, Ch. 9, 13 and 16, John Wiley & Sons; Roe, B., Crabtree, J. and Kahn, A. (1996) DNA Isolation and Sequencing: Essential Techniques, John Wiley & Sons; Polak, J. M. and McGee, J. O'D. (1990) In Situ Hybridization: Principles and Practice, Oxford University Press; Gait, M. J. (1984) Oligonucleotide Synthesis: A Practical Approach, IRL Press; and Lilley, D. M. and Dahlberg, J. E. (1992) Methods in Enzymology: DNA Structures Part A: Synthesis and Physical Analysis of DNA, Academic Press. Each of these general texts is herein incorporated by reference.

### EXAMPLES

### Example 1

### Results

Low density lipoprotein receptor (LDLR) has been reported to be a receptor for VSV.G pseudotyped lentiviral vector (LV). As a result, there was a question whether VSV.G LV encoding LDLR could be difficult to generate, because of a possible interaction between LDLR and VSV.G, impeding the correct packaging of the viral particle. To test this, we produced vectors by transfecting 293T cells. We produced VSV.G LV encoding LDLR or GFP. Infectious titer of each LV preparation was measured by transducing 293T cells. We observed a considerable 10 fold drop in infectious titer, physical particles and infectivity (FIG. 1A) of LV encoding LDLR, compared to LV encoding GFP. This may implicate an interaction between the receptor and VSV.G, or a re-infection of the producer cells due to the exposure of the receptor on the membrane of the cells, resulting in the accumulation of LV inside the cell at the expense of the amount in the supernatant. This was occurring despite the presence of a hepatocyte specific promoter upstream the LDLR. Since 293T cells cannot read this promoter, this implies the expression of the LDLR was resulting from CMV promoter upstream the long terminal repeats (LTR) of the lentiviral plasmid (FIG. 1B). To understand if the issue was due to the expression of LDLR during LV production, we flipped the cassette, in order to have the transgene in reverse orientation (FIG. 1B). Using this kind of construct, the expression of the transgene product is avoided during LV production but guaranteed once the LV is integrated into the target cells which, being hepatocytes, will be able to read the hepato-specific promoter sequence. We observed a rescue in infectious titer: LDLR encoding VSV.G LV were comparable to a reporter factor IX (FIX) transgene encoding VSV.G LV (FIG. 1C).

To verify if LDLR was efficiently expressed in target cells, despite being in reverse orientation, we transduced Huh7 (human hepatoma cell line) (FIG. 1D) and Hepa1.6 (murine hepatoma cell line) (FIG. 1E) with ET.hLDLR sense oriented and ET.hLDLR reverse oriented at increasing multiplicity of infection (MOI 1, 10, 30). We detected almost as high LDLR expression in cells transduced with reverse oriented cassette as in cells transduced with sense oriented cassette, at similar vector DNA copies per cell (vector copy number, VCN). We assessed this by looking at percentage of LDLR+ cells and mean fluorescence intensity (MFI) by flow cytometry. Despite Huh7 human cells expressing human LDLR, we were able to appreciate upregulation of MFI at increasing MOI.

Once the issue of low infectious titer was solved, we moved to the possible issue related to the animal model: reduced efficiency of gene transfer in mice lacking LDLR. To directly compare the in vivo liver transduction efficiency of Ldlr^{-/-} mice, we treated Ldlr¹ and C57 mice (n=5 per group) with LV, pseudotyped with VSV.G, encoding FIX reporter, to monitor longitudinally the transgene output in these mice (FIG. 2A). Surprisingly, mice lacking LDLR showed >2.5 fold higher circulating FIX amounts, compared to mice with functional LDLR. The difference was maintained throughout the experiment, until 3 months post gene therapy (FIG. 2B). At the end of experiment, mice were perfused, and liver subpopulations sorted. VCN on sorted hepatocytes confirmed the higher gene transfer in mice lacking LDLR (FIG. 2C). VCN in LSEC of Ldlr^{-/-} mice was higher than in C57, while VCN in KC was comparable between the two strains (FIG. 2D, E).

We performed a gene therapy experiment in Ldlr^{-/-} mice, displaying clinical and biochemical features of FH (LDL levels >5 fold higher than wild type mice), even though at lower extent compared to human beings (atherosclerosis is absent). We treated 12 juvenile mice with LDLR LV at a dose of 4E10 TU/kg. We followed them for 6 weeks (FIG. 3A). All mice treated with LV showed, in the weeks following gene therapy, a decrease in total cholesterol and LDL cholesterol levels, leading to full normalization (FIG. 3B, C). Starting from 6 weeks after gene therapy, 6 treated mice, 5 Ldlr^{-/-} untreated age matched controls and 5 C57 untreated age matched controls started a challenge of high fat high cholesterol diet (western diet), required when approaching a metabolic disease. The challenge lasted 3 months. Total and LDL cholesterol remained controlled overtime in the treated mice, without showing the escalation in levels experienced by Ldlr^{-/-} untreated mice (FIG. 3D, E). Following discontinuation of western diet, all the mice went back to their values pre-challenge. The mice, treated with LV and normalized in their total and LDL cholesterol levels, that were not subjected to western diet challenge, did not show any increase in total and LDL cholesterol throughout the 6 months of follow up. At the end of experiment, 100% of the mice Ldlr^{-/-} fed with western diet (FIG. 4A) showed atherosclerosis at the levels of aortic sinus (FIG. 4B, C), aortic arch (FIG. 4D) and thoracic aorta (FIG. 4E). None of the C57 normal mice fed with western diet showed atherosclerosis. Importantly, also no Ldlr^{-/-} mice treated with LV and fed with western diet showed atherosclerosis. Minimal to mild atherosclerosis was observed in the Ldlr^{-/-} mice kept under normal diet throughout the experiment, while no atherosclerosis was observed in C57 mice or in Ldlr^{-/-} mice treated with LV.

We performed a long-term gene therapy experiment in Ldlr^{-/-} mice. We treated 7 juvenile mice with LDLR LV at a dose of 4E10 TU/Kg. We followed them for 1 year (FIG. 5A). All mice treated with LV showed a decrease in total and LDL cholesterol levels, leading to full normalization, which was stable and maintained throughout the 1 year follow up (FIG. 5B, C), highlighting stability of the therapeutic effect in mice treated as juvenile and lack of counter-selection of hepatocytes expressing LDLR.

We then assessed the feasibility of keeping the transgene in sense orientation. To do so, we had to avoid the interaction between VSV.G and LDLR overexpressed by LV producer cells during LV production. PCSK9 is the natural inhibitor of LDLR, thus we speculated that addition of PCSK9 during LV production may rescue the titer. The assumption was the following: PCSK9 interacts with LDLR, mediating its degradation before its exposure on the membrane of the cells, thus reducing the interaction between LDLR and VSV.G and the re-infection of producer cells by LV released in the supernatant. To avoid PCSK9 accumulation in the supernatant of producer cells and its co-injection together with the LV in vivo, we used a non-secreted GOF PCSK9 variant, reported to be responsible for FH. We transfected 293T cells with standard LV plasmids mix, with or without the addition of PCSK9 encoding plasmid. Importantly, infectious titer showed to be 5-fold higher when PCSK9 plasmid was added to the mix of transfection of LDLR encoding LV, compared to standard transfection (FIG. 6A).

To show therapeutic effect by treating Ldlr^{-/-} mice, we produced hLDLR encoding LV, adding PCSK9 in the transfection mix. Since expected interaction between murine LDL and human LDLR is lower compared to mLDLR, we treated 2-week-old mice at therapeutic dose observed with previous experiments using mLDLR, and at a higher dose (FIG. 6B). Total and LDL cholesterol circulating in the serum were then monitored longitudinally (FIG. 6C, D). Importantly, all mice at the higher dose showed full normalization of total cholesterol and LDL-C, while mice at the lower dose showed intermediate phenotype. Total cholesterol and LDL-C were stable over time, until the last time point analysed (8 months post LV).

Since metabolic diseases may need a high percentage of transduction of the liver tissue to be corrected, we looked for procedures allowing higher gene transfer into hepatocytes. Increasing the potency of hepatocyte gene transfer following in vivo gene therapy would aid in i) reducing the doses to be administered and to ii) face liver diseases where a majority of liver mass may need to be corrected for therapeutic purposes. With the purpose of finding safe and promising enhancers of transduction, we focused on anti-viral pathways, possibly responsible for reduced efficiency of entry, retro-transcription, integration of LV. Bortezomib is a proteasome inhibitor with anti-inflammatory properties. It may decrease KC activation and improve transduction in specific cell types. Marl is a mAb against IFNARI. Since this signal is important for the activation of innate immune response, its administration before LV administration might increase gene transfer potency by reducing viral sensing. To test these two compounds, we used a transgene applied to a disease for which LV dose reduction would be important: hemophilia A (HA). Indeed, we used hFVIII as transgene, since it gives the double advantage of i) making possible longitudinal measurements and ii) representing a challenging transgene for gene therapy, since its sequence is particularly long, LV carrying its sequence display quite low infectious titers and high doses are requested to achieve phenotypic correction. We injected adult Alb-F8*R593C mice i.v. with Bortezomib (1 mg/kg) or saline one-hour before c.o.hFVIII-BDD LV administration (FIG. 7A). We then compared the circulating secreted hFVIII amounts in mice pre-treated with Bortezomib with those of mice treated at the same LV dose but without Bortezomib pre-treatment. Mice pre-treated with Bortezomib showed higher circulating hFVIII amounts, compared to the group receiving exclusively LV. The advantage was maintained throughout the experiment (FIG. 7B). We then compared the VCN in total liver, and sorted liver cell subpopulations detected in Bortezomib pre-treated mice with the ones detected in mice injected with the same LV dose but without Bortezomib pretreatment (FIG. 7C). We observed a trend of decrease in VCN of KC and pDC of Bortezomib pre-treated mice, possibly indicating reduced capture of LV by phagocytes or toxicity in those cells. However, no increase was observed in sorted hepatocytes.

We then moved to Marl. We injected adult Alb-F8*R593C mice with Marl (1 mg/mouse) or saline 3 hours prior LV co.hFV///-BDD administration (FIG. 7D). We compared the circulating secreted hFVIII amounts in mice pre-treated with Marl with the ones circulating in mice treated at the same dose of LV without pre-treatment with Marl. hFVIII circulating amounts were higher in Marl pre-treated mice, throughout the experiment (FIG. 7E). We then compared the VCN in total liver, and sorted liver cell subpopulations detected in Marl pre-treated mice with the ones detected In mice injected with the same LV dose (FIG. 7F). We detected higher VCN in sorted hepatocytes, showing improvement of transduction and reflecting the higher hFVIII transgene output trend throughout the experiment.

Given the increased transgene output and transduction efficiency observed with the FVIII transgene, we decided to test Marl and Bortezomib in combination, to evaluate if the administration of both prior LV could enhance the potency of liver gene transfer more than the two treatments tested separately. Adult mice were injected either with Marl three hours before, with Bortezomib one hour before or with the combination of the two before injection of LV. Blood samples were then collected overtime to assess hFIX circulating amounts FIG. 8A). We confirmed hFIX amounts of Bortezomib pre-treated mice to be higher than controls. However, while we observed a trend of increase in the Marl treated group, the advantage appeared to be less prominent. Importantly, the combination of the two treatments before LV administration did not give any additional advantage, suggesting that Bortezomib could have an effect even on reducing IFN signal, thus explaining its higher beneficial effect on gene therapy, compared to Marl, and the lack of additive/synergistic effect when used in combination (FIG. 8B).

We then sorted the liver cell subpopulations. Bortezomib pre-treated mice showed a skewing in LV subpopulation targeting, with a reduced VCN in KC (FIG. 8E) and pDC (FIG. 8F), a trend of increase in hepatocytes (FIG. 8C) and an increase in LSEC (FIG. 8D).

Fasting was reported as a procedure leading to higher transgene output, following AAV gene therapy, possibly due to the activation of autophagy. Despite the differences existing between AAV and LV, we decided to test this procedure in the context of in vivo liver-directed LV gene therapy.

To test if fasting had an impact on LV transduction, we fasted C57 mice for 24 hours. To visualize a possible higher transgene positive area in fasted mice, we then injected i.v. the mice with a LV encoding hFIX-IRES-GFP, to monitor longitudinally hFIX circulating amounts and look at the liver sections at the end of experiment (FIG. 9A). We observed higher transgene output for fasted mice compared to controls (FIG. 9B). Moreover, percentage of tissue positive area (FIG. 9C) and RNA derived from LV (FIG. 9D) were higher in fasted mice, compared to non-fasted counterparts, reflecting a higher gene transfer in fasted mice. In total liver, VCN was comparable between the two groups (FIG. 9E).

In an effort to understand the reasons behind higher gene transfer in fasted mice, we then decided to monitor mice during fasting, and to look at LDLR level of expression. Groups in the experiment were the following: i) mice used as control and fed ad libidum, ii) mice fasted 12 hours and then euthanized, iii) mice fasted 24 hours and then euthanized, iv) mice fasted 24 hours, re-fed for additional 24 hours and then euthanized (FIG. 10A). We then looked at the liver of experimental mice and performed gene expression analysis on Ldlr. We found Ldlr RNA amounts to be reduced over time over fasting (FIG. 10B). Put together, the higher gene transfer observed in Ldlr^{-/-} mice and reduction of Ldlr over fasting, condition again related to higher transgene output, may suggest that lower amounts of LDLR improve rather than impair LV transduction in vivo in the liver. LDLR could act as a scavenger receptor for LV, with another receptor, present on Ldlr^{-/-} mice just as in C57 mice, whose levels are not impacted by fasting, being a better receptor.

Once identified three different enhancers of transduction (Marl, Bortezomib, fasting), which proved to be effective with different transgenes tested, we wondered if the three transduction enhancers had an additive or even synergistic effect when combined. Since we were also interested in comparing/confirming the advantage of these enhancers in the Ldlr^{-/-} mouse model, we used C57 and Ldlr^{-/-} divided in the following experimental groups (Table 1).

According to the groups, mice were either faster for 24 hours, injected with Marl 3 hours before LV and/or injected with Bortezomib 1-hour before LV. Circulating hFIX amounts were then monitored longitudinally (FIG. 11A).

TABLE 1

| | C57 experimental groups | | | | | | | Ldlr^{-/-} experimental groups | | | | | | |
|---|---|---|---|---|---|---|---|---|---|---|---|---|---|---|
| | I | II | III | IV | V | VI | VII | I | II | III | IV | V | VI | VII |
| LV | x | x | x | x | x | x | x | x | x | x | x | x | x | x |
| Marl | | x | | | x | | x | | x | | | x | | x |
| Bort | | | x | | | x | x | | | x | | | x | x |
| Fast- | | | | x | x | x | x | | | | x | x | x | x |
| ing | | | | | | | | | | | | | | |

In C57, compared to LV only, Marl and Bortezomib, as single treatments before LV administration enhanced transgene output (FIG. 11B, C). The best outcome following single enhancer treatment was however reached with fasting, where transgene amounts were 7.5-fold higher than in control mice treated with LV alone. Moreover, combination of Marl and fasting and combination of Bortezomib and fasting proved to induce an additional advantage in terms of transgene output, 9.5-fold higher and 11-12 fold higher than LV controls, respectively.

Moving to Ldlr^{-/-}, firstly we confirmed the higher transgene output in Ldlr^{-/-}, compared to C57. Moreover, while Marl and Bortezomib showed to give an advantage, the effect of fasting was minimal/mild. Even when combined with fasting, Marl and Bortezomib showed milder increase in Ldlr^{-/-} than C57, compared to their use as single treatments.

The greatest advantage, for Ldlr^{-/-} mice, was achieved with the combination of fasting and Bortezomib, with an overall increase in transgene output of 4.5-6-fold. These data show the great potential of a minimally invasive combination of treatments, which could be combined with LV for ensuring a gene therapy with higher potency.

### Materials and Methods

### Plasmid Construction

The LDLR coding sequences (human or murine) used in the study were synthesized by GeneScript and cloned into a third-generation self-inactivating (SIN) LV transfer plasmid (Milani et al. (2019) Sci Transl Med. 11: eaav7325) under the control of the enhanced transthyretin promoter (ET) or alpha-1-anti-trypsin promoter (HAAT). PCSK9 encoding plasmid was generated by gene synthesis and cloning into pMAX backbone, generating pMAX-PCSK9.

### Vector Production

Lab-grade VSV.G-pseudotyped third-generation SIN LV were produced by calcium phosphate transient transfection into 293T cells. 293T cells were transfected with a solution containing a mix of the selected LV genome transfer plasmid, the packaging plasmids pMDLg/pRRE and pCMV.REV, pMD2.VSV.G and pAdvantage, as previously described (Milani et al. (2017) EMBO Mol Med 9: 1558-1573; Milani et al. (2019) Sci Transl Med. 11: eaav7325). Medium was replaced 14-16 hours post transfection and supernatant was collected around 30 hours after medium change. LV-containing supernatants were sterilized through a 0.22 μm filter (Millipore). Ex vivo LV infectivity tests were performed using non-concentrated LVs. For ex vivo hepatoma cell lines experiments and in vivo experiments, LV containing supernatants were transferred into sterile poliallomer tubes (Beckman) and centrifuged at 20,000 g for 120 min at 20° C. (Beckman Optima XL-100K Ultracentrifuge). LV pellet was resuspended in the appropriate volume of PBS to allow 500-1000× concentration.

Concerning SIN LV encoding LDLR where the transgene and the promoter were kept in sense orientation in the LV, the mix of transfection was modified as follows. 293T cells were transfected with a solution containing a mix of the selected LV genome transfer plasmid, the packaging plasmids pMDLg/pRRE, pCMV.REV, pMD2.VSV.G, pAdvantage and pMAX.PCSK9.

### Lv Titration

For LV titration, 1×10⁵ 293T cells were transduced with serial LV dilutions in the presence of polybrene (8 μg/ml). Genomic DNA (gDNA) was extracted 14 days after transduction, using Maxwell 16 Cell DNA Purification Kit (Promega), following the manufacturer's instructions. VCN was determined by ddPCR, starting from 5-20 ng of template gDNA using primers (HIV fw: 5′-T ACTGACGCTCTCGCACC-3′; HIV rv: 5′-TCTCGACGCAGGACTCG-3′) and a probe (FAM 5′-ATCTCTCTCCTTCTAGCCTC-3′) designed on the primer binding site region of LV. The amount of endogenous DNA was quantified by a primers/probe set designed on the human GAPDH gene (Applied Biosystems HS00483111_cm). The PCR reaction was performed with each primer (900 nM) and the probe (250 nM, 500 nM for Telo) following the manufacturer's instructions (Biorad), read with QX200 reader and analyzed with QuantaSoft software (Biorad). Infectious titer, expressed as TU/mL, was calculated using the formula TU/mL=(VCN×100,000×(1/dilution factor). LV physical particles were measured by HIV-1 Gag p24 antigen immunocapture assay (Perkin Elmer) following the manufacturer's instructions. LV specific infectivity was calculated as the ratio between infectious titer and physical particles.

### Cell Culture and In Vitro Transduction Experiments

HuH7 cells and Hepa1.6 cells were maintained under 37° C., 5% CO₂ condition in Iscove's Modified Dulbecco's Medium (IMDM) supplemented with 10% FBS (Thermo Fisher Scientific, Waltham, MA). Cells were seeded into 6-well plates (1.5×10⁵ cells/well) and transduced with LV variants at different MOI. Ten days post-LV transduction, cells were harvested and vector copy number was measured.

### Flow Cytometry

Flow cytometry analyses were performed using a FACSCanto analyzer (BD Biosciences), equipped with DIVA Software. Between 100,000-500,000 cells were harvested, washed with PBS or MACS buffer (PBS pH 7.2 0.5% BSA, 2Mm EDTA). Staining was performed in MACS buffer, incubating cells with antibody (1:10, staining in 100 uL) for 20 minutes at 4° C. in the dark. Anti-human LDLR PE (R&D, FAB2148A).

### Mice Experiments

All animal experiments were performed in strict accordance with good animal practices following Italian and European legislation on animal care and experimentation (2010/63/EU). Wild-type C57Bl/6 adults, Ldlr/adults, Alb-F8*R593C adults, Ldlr^{-/-} 2-week old mice and C57 BI/6 2-week old were used in these studies. Founder B6; 129S-7-Ldlrtm1 Her/J mice (referred to as Ldlr^{-/-}) were obtained from the Jackson Laboratories (stock #:002207). Founder F8^{tm1Kaz}Tg(Alb-F8*R593C)T4Mcal/J mice (referred to as HemoA-R593C) were obtained from The Jackson Laboratories (stock #017706). C57BL/6 mice were purchased from Jackson laboratories (stock #000664). All mice were kept in specific pathogen free conditions. LV was administered in adult mice, males and females (7-10-weeks of age), through either tail vein or retro-orbital plexus (250-500 μL/mouse). LV was administered in juvenile mice (2 weeks of age) through retro-orbital plexus (100-200 μL/mouse). Mice were bled from the retro-orbital plexus through capillary tubes. Blood was collected in 0.38% sodium citrate buffer, pH 7.4. Mice were humanely killed by cervical dislocation at the scheduled time. All the procedures performed on mice were approved by Institutional Animal Care and Use Committee. For fasting experiments, mice were starved for 24-hours and then treated or not with LV. For experiments with Bortezomib (Velcade), the drug (1 mg/kg) was administered, once diluted in saline, i.v. 1 hour before LV administration. For experiments with Marl (antiIFNARI Ab, clone MARI-5A3, Merk), the drug (50 mg/kg) was administered, once diluted in saline, i.v. 3 hours before LV administration.

### Dietary Regimens

Starting from 4 weeks of age, all mice were fed ad libitum with VRF1 (P) by Special Diet Services. For the experiment with Western Diet Challenge, mice started being fed ad libitum with Envigo TD.88137 (0.2% total cholesterol) for three months. After that, they were put back on VRF1. For fasting experiments, mice were starved for the number of hours indicated and then treated or not with LV.

### Plasmatic Cholesterol and LDL Quantification

Blood samples were collected via the retro-orbital plexus in 0.5M EDTA-filled tubes. Serum was obtained following blood centrifugation (5500 rpm×15 minutes) and stored at −80° C. Cholesterol ((#0018250540) and LDL Cholesterol (#0018256040) were used for the quantitative determination of the serum level with an International Federation of Clinical Chemistry and Laboratory Medicine optimized kinetic ultraviolet (UV) method in an ILab650 chemical analyser (Instrumentation Laboratory), and there are expressed as mg/dl. SeraChem Control Level 1 and Level 2 (#0018162412 and #0018162512) were analyzed as quality control.

### Fractionation and Sorting of Liver Cell Sub-Populations

The liver was perfused (2.5 mL/min) via the inferior vena cava with 12.5 mL of the following solutions at subsequent steps: 1) PBS EDTA (0.5 mM), 2) HBSS (Hank's balanced salt solution, Gibco) and HEPES (10 mM), 3) HBSS-HEPES 0.03% Collagenase IV (Sigma). The digested liver tissue was harvested, passed through a 70 μm cell strainer (BD Biosciences) and processed into a single-cell suspension. This suspension was subsequently centrifuged three times (30, 25 and 20 g, for 3 minutes, at room temperature) to obtain PC-containing pellets. The nPC-containing supernatant was centrifuged (650 g, 7 minutes, at room temperature) and recovered cells were loaded onto a 30/60% Percoll (Sigma) gradient (1800 g, for 20 minutes at room temperature). nPC interface was collected and washed twice. The nPC were subsequently incubated with the following monoclonal antibodies: e-fluor 450-conjugated anti-CD45 (30-F11, e-Bioscience), Allophycocyanin (APC)-conjugated anti-CD31 (MEC13.3, BD Biosciences), phycoerythrin (PE)-conjugated F4/80 (CI:A3-1, Biorad), PE-Cy5-conjugated anti-CD45R/B220 (from BD Biosciences), PE-Cy7-conjugated anti-CD11c (N418, e-Bioscience), purified anti-CD16/32 (2.4G2, BD Biosciences). nPC subpopulations (LSEC, KC, pDC) were sorted by FACS, MOFLO-DAKO-Beckman-Coulter; the nPC contaminating the PC suspension, were removed by FACS excluding cells labeled by APC-conjugated anti-CD31/anti-CD45 cocktail, thus obtaining sorted hepatocytes (Hep).

### VCN Determination

DNA was extracted from cells or liver samples using Maxwell 16 Cell or Tissue DNA Purification Kits (Promega). VCN was determined in Huh7 as described above (see “LV titration”). VCN in Hepa1.6 was determined as described above (see “LV titration”); but the amount of endogenous murine DNA was quantified by a primers/probe set designed on the murine sema3a gene (Sema3A fw: 5′-ACCGATTCCAGATGATTGGC-3′; Sema3A rv: 5′-TCCATATTAATGCAGTGCTTGC-3′; Sema3A probe: HEX 5′-AGAGGCCTGTCCTGCAGCTCATGG-3′ BHQ1). VCN in murine DNA was determined by ddPCR, starting from 5-20 ng of template gDNA using a primers/probe set designed on the primer binding site region of LV (see “LV titration” above), using again sema3a gene as endogenous murine DNA quantifier. The PCR reaction was performed with each primer (900 nM) and the probe (250 nM) following the manufacturer's instructions (Biorad), read with QX200 reader and analyzed with QuantaSoft software (Biorad).

### Gene Expression Assays

Murine organs, murine primary sorted hepatocytes and LSEC were stored at −80° C. in RLT+ buffer (Qiagen) solution, suggested for RNA extraction. In case of organs, 300 μl of RLT+ were added every 25 mg tissue piece. In case of cells, 250 μl of RLT+ were added by default. Organs were homogenized using gentleMACS™ M Tubes (MACS Miltenyi Biotec, 130096335). Tubes were inserted in gentleMACS™ Octo Dissociator (Miltenyi Biotec) and protocol selected was gentleMACS program RNA_02, suggested for frozen tissues. Homogenized tissues were subsequently loaded into Maxwell® RSC simplyRNA tissue cartridges and RNA was extracted using simplyRNA tissue method. Retro transcription was performed using SuperScript IV VILO Master Mix with EzDNase Enzyme (Thermo Fisher), in accordance with manufacturer's instruction. Each sample was retro-transcribed in two wells, one containing the enzyme (RT+), and one without the enzyme (RT−). cDNA was analyzed by ddPCR as described above, using probe systems. RT− signal was subtracted from RT+ signal, resulting in true signal. Commercially available primers and probes were used. As normalizer, commercial Hprt primers and probe were used. Gene expression levels were calculated with the formula ng cDNA gene/ng cDNA normalizer. Commercial Biorad gene expression assays used were the following:
Ldlr Mus Musculus, dMmuCPE5122114, FAM
Hprt Mus Musculus, dMmuCPE5095493, VIC

### Collection of Aorta and Histopathology Analysis

Mice were humanely killed and then perfused via left ventricle with the following solutions at subsequent steps: 1) PBS EDTA (0.5 mM), 15 mL 2) PFA (Paraformaldehyde). Aorta was subsequently dissected, and the tissue covering aorta removed. Mice aortas were then formalin-fixed and paraffin-embedded, cross-sectioned and stained with hematoxylin and eosin.

### FIX ELISA Assay

Mouse blood was centrifuged at 6500 rpm for 6.5 minutes for plasma collection, then stored at −80° C. The concentration of human FVIII was determined in mouse plasma by an enzyme-linked immunosorbent assay (ELISA) specific for human FVIII antigen. Microtiter plates were coated with anti-hFVIII binding Ab (Green Mountain Antibodies #GMA8016, 0.2 μg/well in 0.1 M carbonate buffer, pH 9.6) over night at 4° C. and then blocked 1 hour at room temperature with blocking buffer (PBS 0.05% Tween-20, 1M NaCl, 10% heat inactivated horse serum, Gibco). Plasma samples are diluted as needed starting from 1:10 in blocking buffer, added to wells (100 μL/well) and incubated 2 hours at 37° C. hFVIII was detected by adding detection Ab (Affinity Biologicals, F8C-EIC-D) 1 hour at 37° C., followed by 5-10 minutes incubation with 100 μL/well of TMB substrate (Surmodics). Reaction was blocked with HCl 1N (50 μL/well) and absorbance of each sample was determined spectrophotometrically at 450 nm, using a Multiskan GO microplate reader (Thermo Fisher Scientific) and normalized to antigen standard curve (ReFACTO, Pfizer, from 25 ng/mL to 0.39 ng/mL serially diluted 1:2 in blocking buffer; dilution was corrected with 10% HemoA murine plasma).

hFIX ELISA assay was used to assess hFIX concentration in mouse plasma samples (Affinity Biologicals, FIX-EIA), in accordance with the manufacturer's protocol. Absorbance of each sample was determined spectrophotometrically, using a Multiskan GO microplate reader (Thermo Fisher Scientific) and normalized to antigen standard curves.

### Statistical Analysis

Statistical analyses were performed by Prism 9 software.

All publications mentioned in the above specification are herein incorporated by reference. Various modifications and variations of the disclosed vectors, cells, compositions, uses and methods of the invention will be apparent to the skilled person without departing from the scope and spirit of the invention. Although the invention has been disclosed in connection with specific preferred embodiments, it should be understood that the invention as claimed should not be unduly limited to such specific embodiments. Indeed, various modifications of the disclosed modes for carrying out the invention, which are obvious to the skilled person are intended to be within the scope of the following claims.

### Embodiments

Various features and embodiments of the present invention will now be described with reference to the following numbered paragraphs:

1. A lentiviral vector comprising a nucleotide sequence encoding low density lipoprotein receptor (LDLR) operably linked to a liver-specific promoter, optionally wherein the nucleotide sequence encoding LDLR and the promoter are in a reverse orientation in the lentiviral vector.

2. The lentiviral vector of paragraph 1, wherein the LDLR comprises or consists of an amino acid sequence that has at least 70% sequence identity to SEQ ID NO: 1 or 38, preferably SEQ ID NO: 1, or a fragment thereof.

3. The lentiviral vector of paragraph 1 or 2, wherein the nucleotide sequence encoding LDLR comprises or consists of a nucleotide sequence that has at least 70% sequence identity to SEQ ID NO: 2 or 39, preferably SEQ ID NO: 2, or a fragment thereof.

4. The lentiviral vector of any preceding paragraph, wherein the promoter is a liver-specific promoter, optionally a hepatocyte-specific promoter.

5. The lentiviral vector of any preceding paragraph, wherein the promoter is a transthyretin (TTR) promoter, optionally an enhanced transthyretin (ET) promoter.

6. The lentiviral vector of any preceding paragraph, wherein the promoter comprises or consists of a nucleotide sequence that has at least 70% sequence identity to SEQ ID NO: 24 or 25, preferably SEQ ID NO: 24.

7. The lentiviral vector of any preceding paragraph, wherein the lentiviral vector is a VSV.G-pseudotyped lentiviral vector.

8. The lentiviral vector of any preceding paragraph, wherein the lentiviral vector is a self-inactivating (SIN) lentiviral vector.

9. The lentiviral vector of any preceding paragraph, wherein the nucleotide sequence encoding LDLR is operably linked to one or more miRNA target sequence.

10. The lentiviral vector of paragraph 9, wherein the one or more miRNA target sequence suppresses LDLR expression in one or more cell type other than hepatocytes, optionally wherein the one or more miRNA target sequence suppresses LDLR expression in hematopoietic-lineage cells and/or antigen-presenting cells.

11. The lentiviral vector of paragraph 9 or 10, wherein the one or more miRNA target sequence is selected from the group consisting of a miR-142 target sequence, a miR-181 target sequence, a miR-223 target sequence and a miR-155 target sequence.

12. The lentiviral vector of any preceding paragraph, wherein the nucleotide sequence encoding LDLR is operably linked to one or more miR-142 target sequence, two or more miR-142 target sequences, three or more miR-142 target sequences, or four or more miR-142 target sequences.

13. The lentiviral vector of any preceding paragraph, wherein the lentiviral vector is a CD47^{high} lentiviral vector.

14. The lentiviral vector of any preceding paragraph, wherein the lentiviral vector is a MHC-I^{free} lentiviral vector.

15. The lentiviral vector of any preceding paragraph, wherein the lentiviral vector is a CD47^{high}/MHC-I^{free} lentiviral vector.

16. An isolated cell comprising the lentiviral vector of any preceding paragraph.

17. A pharmaceutical composition comprising the lentiviral vector of any one of paragraphs 1-15 or the cell of paragraph 16, and a pharmaceutically acceptable carrier, diluent or excipient.

18. The lentiviral vector of any one of paragraphs 1-15 or the cell of paragraph 16 for use in therapy.

19. The lentiviral vector of any one of paragraphs 1-15 or the cell of paragraph 16 for use in treatment or prevention of familial hypercholesterolemia (FH) or familial hypercholesterolemia associated conditions, such as atherosclerosis.

20. The lentiviral vector or cell for use according to paragraph 18 or 19, wherein the subject is a juvenile.

21. The lentiviral vector or cell for use according to paragraph 18 or 19, wherein the subject is a paediatric subject, optionally wherein the subject is a neonatal subject or an infantile subject.

22. The lentiviral vector or cell for use according to any one of paragraphs 18-21, wherein the lentiviral vector is administered systemically, optionally wherein the lentiviral vector is administered by intravenous injection or intraperitoneal injection.

23. The lentiviral vector or cell for use according to any one of paragraphs 18-22, wherein the lentiviral vector is administered as a single dose or multiple doses.

24. The lentiviral vector or cell for use according to any one of paragraphs 20-23, wherein the lentiviral vector is administered locally to the liver, optionally wherein the lentiviral vector is administered by intrahepatic injection, intrahepatic arterial injection or intraportal injection.

25. The lentiviral vector or cell for use according to any one of paragraphs 18-24, wherein total cholesterol levels are reduced and/or normalised.

26. The lentiviral vector or cell for use according to any one of paragraphs 18-24, wherein LDL cholesterol levels are reduced and/or normalised.

1. A lentiviral vector comprising a nucleotide sequence encoding low density lipoprotein receptor (LDLR) operably linked to a liver-specific promoter.

2. The lentiviral vector of claim 1, wherein the nucleotide sequence encoding LDLR and the promoter are in a reverse orientation in the lentiviral vector.

3. The lentiviral vector of claim 1, wherein the nucleotide sequence encoding LDLR is in a sense orientation in the lentiviral vector.

4. The lentiviral vector of any preceding claim, wherein the LDLR comprises or consists of an amino acid sequence that has at least 70% sequence identity to SEQ ID NO: 1 or 38, preferably SEQ ID NO: 1, or a fragment thereof.

5. The lentiviral vector of any preceding claim, wherein the promoter is a transthyretin (TTR) promoter, optionally an enhanced transthyretin (ET) promoter.

6. The lentiviral vector of any preceding claim, wherein the nucleotide sequence encoding LDLR is operably linked to one or more miR-142 target sequence, two or more miR-142 target sequences, three or more miR-142 target sequences, or four or more miR-142 target sequences.

7. The lentiviral vector of any preceding claim, wherein the lentiviral vector is a CD47^{high}/MHC-I^{free} lentiviral vector.

8. An isolated cell comprising the lentiviral vector of any preceding claim.

9. The lentiviral vector of any one of claims 1-7 or the cell of claim 8 for use in therapy.

10. The lentiviral vector of any one of claims 1-7 or the cell of claim 8 for use in treatment or prevention of familial hypercholesterolemia (FH) or familial hypercholesterolemia associated conditions, such as atherosclerosis.

11. The lentiviral vector or cell for use according to claim 9 or 10, wherein the subject is a juvenile.

12. A viral vector for use in a method of treatment of a subject, wherein:
(a) the method further comprises administering an interferon αβ receptor I (IFNARI) inhibitor to the subject, optionally before the administration of the viral vector;
(b) the method further comprises administering a proteasome inhibitor to the subject, optionally before the administration of the viral vector; and/or
(c) the method further comprises the subject fasting, optionally before the administration of the viral vector.

13. The viral vector for use according to claim 12, wherein the viral vector is a lentiviral vector.

14. The viral vector for use according to claim 12 or 13, wherein the IFNARI inhibitor is an anti-IFNARI antibody, optionally Marl.

15. The viral vector for use according to any one of claims 12-14, wherein the proteasome inhibitor is Bortezomib.


SEQUENCE LISTING (49 sequences)

SEQ ID NO: 1 - protein, 860 aa, synthetic construct [sequence omitted]

SEQ ID NO: 2 - DNA, 2583 bp, synthetic construct
  misc_feature 1..2583: note = Synthetic
  source 1..2583: mol_type = other DNA
atggggccctggggctggaaattgcgctggaccgtcgccttgctcctcgccgcggcggggactgcagtgggcgacagatgcgaaagaaacgagttccagtgccaagacgggaaatgcatctcctacaagtgggtctgcgatggcagcgctgagtgccaggatggctctgatgagtcccaggagacgtgcttgtctgtcacctgcaaatccggggacttcagctgtgggggccgtgtcaaccgctgcattcctcagttctggaggtgcgatggccaagtggactgcgacaacggctcagacgagcaaggctgtccccccaagacgtgctcccaggacgagtttcgctgccacgatgggaagtgcatctctcggcagttcgtctgtgactcagaccgggactgcttggacggctcagacgaggcctcctgcccggtgctcacctgtggtcccgccagcttccagtgcaacagctccacctgcatcccccagctgtgggcctgcgacaacgaccccgactgcgaagatggctcggatgagtggccgcagcgctgtaggggtctttacgtgttccaaggggacagtagcccctgctcggccttcgagttccactgcctaagtggcgagtgcatccactccagctggcgctgtgatggtggccccgactgcaaggacaaatctgacgaggaaaactgcgctgtggccacctgtcgccctgacgaattccagtgctctgatggaaactgcatccatggcagccggcagtgtgaccgggaatatgactgcaaggacatgagcgatgaagttggctgcgttaatgtgacactctgcgagggacccaacaagttcaagtgtcacagcggcgaatgcatcaccctggacaaagtctgcaacatggctagagactgccgggactggtcagatgaacccatcaaagagtgcgggaccaacgaatgcttggacaacaacggcggctgttcccacgtctgcaatgaccttaagatcggctacgagtgcctgtgccccgacggcttccagctggtggcccagcgaagatgcgaagatatcgatgagtgtcaggatcccgacacctgcagccagctctgcgtgaacctggagggtggctacaagtgccagtgtgaggaaggcttccagctggacccccacacgaaggcctgcaaggctgtgggctccatcgcctacctcttcttcaccaaccggcacgaggtcaggaagatgacgctggaccggagcgagtacaccagcctcatccccaacctgaggaacgtggtcgctctggacacggaggtggccagcaatagaatctactggtctgacctgtcccagagaatgatctgcagcacccagcttgacagagcccacggcgtctcttcctatgacaccgtcatcagcagagacatccaggcccccgacgggctggctgtggactggatccacagcaacatctactggaccgactctgtcctgggcactgtctctgttgcggataccaagggcgtgaagaggaaaacgttattcagggagaacggctccaagccaagggccatcgtggtggatcctgttcatggcttcatgtactggactgactggggaactcccgccaagatcaagaaagggggcctgaatggtgtggacatctactcgctggtgactgaaaacattcagtggcccaatggcatcaccctagatctcctcagtggccgcctctactgggttgactccaaacttcactccatctcaagcatcgatgtcaacgggggcaaccggaagaccatcttggaggatgaaaagaggctggcccaccccttctccttggccgtctttgaggacaaagtattttggacagatatcatcaacgaagccattttcagtgccaaccgcctcacaggttccgatgtcaacttgttggctgaaaacctactgtccccagaggatatggttctcttccacaacctcacccagccaagaggagtgaactggtgtgagaggaccaccctgagcaatggcggctgccagtatctgtgcctccctgccccgcagatcaacccccactcgcccaagtttacctgcgcctgcccggacggcatgctgctggccagggacatgaggagctgcctcacagaggctgaggctgcagtggccacccaggagacatccaccgtcaggctaaaggtcagctccacagccgtaaggacacagcacacaaccacccgacctgttcccgacacctcccggctgcctggggccacccctgggctcaccacggtggagatagtgacaatgtctcaccaagctctgggcgacgttgctggcagaggaaatgagaagaagcccagtagcgtgagggctctgtccattgtcctccccatcgtgctcctcgtcttcctttgcctgggggtcttccttctatggaagaactggcggcttaagaacatcaacagcatcaactttgacaaccccgtctatcagaagaccacagaggatgaggtccacatttgccacaaccaggacggctacagctacccctcgagacagatggtcagtctggaggatgacgtggcgtga

SEQ ID NO: 3-5 - skipped (3 entries)

SEQ ID NO: 6 - DNA, 23 bp, synthetic construct
  misc_feature 1..23: note = Synthetic
  source 1..23: mol_type = other DNA
tccataaagtaggaaacactaca

SEQ ID NO: 7 - DNA, 106 bp, synthetic construct
  misc_feature 1..106: note = Synthetic
  source 1..106: mol_type = other DNA
tccataaagtaggaaacactacacgattccataaagtaggaaacactacaaccggttccataaagtaggaaacactacatcactccataaagtaggaaacactaca

SEQ ID NO: 8 - protein, 305 aa, synthetic construct
  REGION 1..305: note = Synthetic
MWPLVAALLLGSACCGSAQLLFNKTKSVEFTFCNDTVVIPCFVTNMEAQNTTEVYVKWKFKGRDIYTFDGALNKSTVPTDFSSAKIEVSQLLKGDASLKMDKSDAVSHTGNYTCEVTELTREGETIIELKYRVVSWFSPNENILIVIFPIFAILLFWGQFGIKTLKYRSGGMDEKTIALLVAGLVITVIVIVGAILFVPGEYSLKNATGLGLIVTSTGILILLHYYVFSTAIGLTSFVIAILVIQVIAYILAVVGLSLCIAACIPMHGPLLISGLSILALAQLLGLVYMKFVASNQKTIQPPRNN

SEQ ID NO: 9 - protein, 323 aa, synthetic construct
  REGION 1..323: note = Synthetic
MWPLVAALLLGSACCGSAQLLFNKTKSVEFTFCNDTVVIPCFVTNMEAQNTTEVYVKWKFKGRDIYTFDGALNKSTVPTDFSSAKIEVSQLLKGDASLKMDKSDAVSHTGNYTCEVTELTREGETIIELKYRVVSWFSPNENILIVIFPIFAILLFWGQFGIKTLKYRSGGMDEKTIALLVAGLVITVIVIVGAILFVPGEYSLKNATGLGLIVTSTGILILLHYYVFSTAIGLTSFVIAILVIQVIAYILAVVGLSLCIAACIPMHGPLLISGLSILALAQLLGLVYMKFVASNQKTIQPPRKAVEEPLNAFKESKGMMNDE

SEQ ID NO: 10 - protein, 292 aa, synthetic construct
  REGION 1..292: note = Synthetic
MWPLVAALLLGSACCGSAQLLFNKTKSVEFTFCNDTVVIPCFVTNMEAQNTTEVYVKWKFKGRDIYTFDGALNKSTVPTDFSSAKIEVSQLLKGDASLKMDKSDAVSHTGNYTCEVTELTREGETIIELKYRVVSWFSPNENILIVIFPIFAILLFWGQFGIKTLKYRSGGMDEKTIALLVAGLVITVIVIVGAILFVPGEYSLKNATGLGLIVTSTGILILLHYYVFSTAIGLTSFVIAILVIQVIAYILAVVGLSLCIAACIPMHGPLLISGLSILALAQLLGLVYMKFV

SEQ ID NO: 11 - protein, 311 aa, synthetic construct
  REGION 1..311: note = Synthetic
MWPLVAALLLGSACCGSAQLLFNKTKSVEFTFCNDTVVIPCFVTNMEAQNTTEVYVKWKFKGRDIYTFDGALNKSTVPTDFSSAKIEVSQLLKGDASLKMDKSDAVSHTGNYTCEVTELTREGETIIELKYRVVSWFSPNENILIVIFPIFAILLFWGQFGIKTLKYRSGGMDEKTIALLVAGLVITVIVIVGAILFVPGEYSLKNATGLGLIVTSTGILILLHYYVFSTAIGLTSFVIAILVIQVIAYILAVVGLSLCIAACIPMHGPLLISGLSILALAQLLGLVYMKFVASNQKTIQPPRKAVEEPLN

SEQ ID NO: 12 - protein, 287 aa, synthetic construct
  REGION 1..287: note = Synthetic
QLLFNKTKSVEFTFCNDTVVIPCFVTNMEAQNTTEVYVKWKFKGRDIYTFDGALNKSTVPTDFSSAKIEVSQLLKGDASLKMDKSDAVSHTGNYTCEVTELTREGETIIELKYRVVSWFSPNENILIVIFPIFAILLFWGQFGIKTLKYRSGGMDEKTIALLVAGLVITVIVIVGAILFVPGEYSLKNATGLGLIVTSTGILILLHYYVFSTAIGLTSFVIAILVIQVIAYILAVVGLSLCIAACIPMHGPLLISGLSILALAQLLGLVYMKFVASNQKTIQPPRNN

SEQ ID NO: 13 - protein, 305 aa, synthetic construct
  REGION 1..305: note = Synthetic
QLLFNKTKSVEFTFCNDTVVIPCFVTNMEAQNTTEVYVKWKFKGRDIYTFDGALNKSTVPTDFSSAKIEVSQLLKGDASLKMDKSDAVSHTGNYTCEVTELTREGETIIELKYRVVSWFSPNENILIVIFPIFAILLFWGQFGIKTLKYRSGGMDEKTIALLVAGLVITVIVIVGAILFVPGEYSLKNATGLGLIVTSTGILILLHYYVFSTAIGLTSFVIAILVIQVIAYILAVVGLSLCIAACIPMHGPLLISGLSILALAQLLGLVYMKFVASNQKTIQPPRKAVEEPLNAFKESKGMMNDE

SEQ ID NO: 14 - protein, 274 aa, synthetic construct
  REGION 1..274: note = Synthetic
QLLFNKTKSVEFTFCNDTVVIPCFVTNMEAQNTTEVYVKWKFKGRDIYTFDGALNKSTVPTDFSSAKIEVSQLLKGDASLKMDKSDAVSHTGNYTCEVTELTREGETIIELKYRVVSWFSPNENILIVIFPIFAILLFWGQFGIKTLKYRSGGMDEKTIALLVAGLVITVIVIVGAILFVPGEYSLKNATGLGLIVTSTGILILLHYYVFSTAIGLTSFVIAILVIQVIAYILAVVGLSLCIAACIPMHGPLLISGLSILALAQLLGLVYMKFV

SEQ ID NO: 15 - protein, 293 aa, synthetic construct
  REGION 1..293: note = Synthetic
QLLFNKTKSVEFTFCNDTVVIPCFVTNMEAQNTTEVYVKWKFKGRDIYTFDGALNKSTVPTDFSSAKIEVSQLLKGDASLKMDKSDAVSHTGNYTCEVTELTREGETIIELKYRVVSWFSPNENILIVIFPIFAILLFWGQFGIKTLKYRSGGMDEKTIALLVAGLVITVIVIVGAILFVPGEYSLKNATGLGLIVTSTGILILLHYYVFSTAIGLTSFVIAILVIQVIAYILAVVGLSLCIAACIPMHGPLLISGLSILALAQLLGLVYMKFVASNQKTIQPPRKAVEEPLN

SEQ ID NO: 16 - protein, 140 aa, synthetic construct
  REGION 1..140: note = Synthetic
MWPLVAALLLGSACCGSAQLLFNKTKSVEFTFCNDTVVIPCFVTNMEAQNTTEVYVKWKFKGRDIYTFDGALNKSTVPTDFSSAKIEVSQLLKGDASLKMDKSDAVSHTGNYTCEVTELTREGETIIELKYRVVSWFSPN

SEQ ID NO: 17 - protein, 122 aa, synthetic construct
  REGION 1..122: note = Synthetic
QLLFNKTKSVEFTFCNDTVVIPCFVTNMEAQNTTEVYVKWKFKGRDIYTFDGALNKSTVPTDFSSAKIEVSQLLKGDASLKMDKSDAVSHTGNYTCEVTELTREGETIIELKYRVVSWFSPN

SEQ ID NO: 18 - protein, 365 aa, synthetic construct
  REGION 1..365: note = Synthetic
MAVMAPRTLLLLLSGALALTQTWAGSHSMRYFFTSVSRPGRGEPRFIAVGYVDDTQFVRFDSDAASQRMEPRAPWIEQEGPEYWDQETRNVKAQSQTDRVDLGTLRGYYNQSEAGSHTIQIMYGCDVGSDGRFLRGYRQDAYDGKDYIALNEDLRSWTAADMAAQITKRKWEAAHEAEQLRAYLDGTCVEWLRRYLENGKETLQRTDPPKTHMTHHPISDHEATLRCWALGFYPAEITLTWQRDGEDQTQDTELVETRPAGDGTFQKWAAVVVPSGEEQRYTCHVQHEGLPKPLTLRWELSSQPTIPIVGIIAGLVLLGAVITGAVVAAVMWRRKSSDRKGGSYTQAASSDSAQGSDVSLTACKV

SEQ ID NO: 19 - protein, 371 aa, synthetic construct
  REGION 1..371: note = Synthetic
MAVMAPRTLLLLLSGALALTQTWAGSHSMRYFFTSVSRPGRGEPRFIAVGYVDDTQFVRFDSDAASQRMEPRAPWIEQEGPEYWDQETRNVKAQSQTDRVDLGTLRGYYNQSEAGSHTIQIMYGCDVGSDGRFLRGYRQDAYDGKDYIALNEDLRSWTAADMAAQITKRKWEAAHAAEQQRAYLEGRCVEWLRRYLENGKETLQRTDPPKTHMTHHPISDHEATLRCWALGFYPAEITLTWQRDGEDQTQDTELVETRPAGDGTFQKWAAVVVPSGEEQRYTCHVQHEGLPKPLTLRWELSSQPTIPIVGIIAGLVLLGAVITGAVVAAVMWRRKSSGGEGVKDRKGGSYTQAASSDSAQGSDVSLTACKV

SEQ ID NO: 20 - protein, 362 aa, synthetic construct
  REGION 1..362: note = Synthetic
MLVMAPRTVLLLLSAALALTETWAGSHSMRYFYTSVSRPGRGEPRFISVGYVDDTQFVRFDSDAASPREEPRAPWIEQEGPEYWDRNTQIYKAQAQTDRESLRNLRGYYNQSEAGSHTLQSMYGCDVGPDGRLLRGHDQYAYDGKDYIALNEDLRSWTAADTAAQITQRKWEAAREAEQRRAYLEGECVEWLRRYLENGKDKLERADPPKTHVTHHPISDHEATLRCWALGFYPAEITLTWQRDGEDQTQDTELVETRPAGDRTFQKWAAVVVPSGEEQRYTCHVQHEGLPKPLTLRWEPSSQSTVPIVGIVAGLAVLAVVVIGAVVAAVMCRRKSSGGKGGSYSQAACSDSAQGSDVSLTA

SEQ ID NO: 21 - protein, 366 aa, synthetic construct
  REGION 1..366: note = Synthetic
MRVMAPRALLLLLSGGLALTETWACSHSMRYFDTAVSRPGRGEPRFISVGYVDDTQFVRFDSDAASPRGEPRAPWVEQEGPEYWDRETQKYKRQAQADRVSLRNLRGYYNQSEDGSHTLQRMSGCDLGPDGRLLRGYDQSAYDGKDYIALNEDLRSWTAADTAAQITQRKLEAARAAEQLRAYLEGTCVEWLRRYLENGKETLQRAEPPKTHVTHHPLSDHEATLRCWALGFYPAEITLTWQRDGEDQTQDTELVETRPAGDGTFQKWAAVVVPSGQEQRYTCHMQHEGLQEPLTLSWEPSSQPTIPIMGIVAGLAVLVVLAVLGAVVTAMMCRRKSSGGKGGSCSQAACSNSAQGSDESLITCKA

SEQ ID NO: 22 - protein, 326 aa, synthetic construct
  REGION 1..326: note = Synthetic
MRVMAPRALLLLLSGGLALTETWACSHSMRYFDTAVSRPGRGEPRFISVGYVDDTQFVRFDSDAASPRGEPRAPWVEQEGPEYWDRETQKYKRQAQADRVSLRNLRGYYNQSEDGSHTLQRMSGCDLGPDGRLLRGYDQSAYDGKDYIALNEHLRSCTAADTAAQITQRKLEAARAAEQLRAYLEGTCVEWLRRYLENGKETLQRAEPPKTHVTHHPLSDHEATLRCWALGFYPAEITLTWQRDGEDQTQDTELVETRPAGDGTFQKWAAVVVPSGQEQRYTCHMQHEGLQEPLTLRWGGKGGSCSQAACSNSAQGSDESLITCKA

SEQ ID NO: 23 - protein, 119 aa, synthetic construct
  REGION 1..119: note = Synthetic
MSRSVALAVLALLSLSGLEAIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDM

SEQ ID NO: 24 - DNA, 553 bp, synthetic construct
  misc_feature 1..553: note = Synthetic
  source 1..553: mol_type = other DNA
cgcgagttaataattaccagcgcgggccaaataaataatccgcgaggggcaggtgacgtttgcccagcgcgcgctggtaattattaacctcgcgaatattgattcgaggccgcgattgccgcaatcgcgaggggcaggtgacctttgcccagcgcgcgttcgccccgccccggacggtatcgataagcttaggagcttgggctgcaggtcgagggcactgggaggatgttgagtaagatggaaaactactgatgacccttgcagagacagagtattaggacatgtttgaacaggggccgggcgatcagcaggtagctctagaggatccccgtctgtctgcacatttcgtagagcgagtgttccgatactctaatctccctaggcaaggttcatatttgtgtaggttacttattctccttttgttgactaagtcaataatcagaatcagcaggtttggagtcagcttggcagggatcagcagcctgggttggaaggagggggtataaaagccccttcaccaggagaagccgtcacacagatccacaagctcctg

SEQ ID NO: 25 - DNA, 347 bp, synthetic construct
  misc_feature 1..347: note = Synthetic
  source 1..347: mol_type = other DNA
gatcttgctaccagtggaacagccactaaggattctgcagtgagagcagagggccagctaagtggtactctcccagagactgtctgactcacgccaccccctccaccttggacacaggacgctgtggtttctgagccaggtacaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacag

SEQ ID NO: 26 - DNA, 504 bp, synthetic construct
  misc_feature 1..504: note = Synthetic
  source 1..504: mol_type = other DNA
aatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgc

SEQ ID NO: 27 - skipped

SEQ ID NO: 28 - DNA, 235 bp, synthetic construct
  misc_feature 1..235: note = Synthetic
  source 1..235: mol_type = other DNA
tggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcag

SEQ ID NO: 29 - DNA, 182 bp, synthetic construct
  misc_feature 1..182: note = Synthetic
  source 1..182: mol_type = other DNA
gggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcag

SEQ ID NO: 30 - DNA, 154 bp, synthetic construct
  misc_feature 1..154: note = Synthetic
  source 1..154: mol_type = other DNA
tggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagag

SEQ ID NO: 31 - DNA, 365 bp, synthetic construct
  misc_feature 1..365: note = Synthetic
  source 1..365: mol_type = other DNA
atgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgat

SEQ ID NO: 32 - skipped

SEQ ID NO: 33 - DNA, 241 bp, synthetic construct
  misc_feature 1..241: note = Synthetic
  source 1..241: mol_type = other DNA
ggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggattt

SEQ ID NO: 34 - DNA, 124 bp, synthetic construct
  misc_feature 1..124: note = Synthetic
  source 1..124: mol_type = other DNA
aacttttaaaagaaaaggggggattggggggtacagtgcaggggaaagaatagtagacataatagcaacagacatacaaactaaagaattacaaaaacaaattacaaaaattcaaaattttatc

SEQ ID NO: 35 - DNA, 146 bp, synthetic construct
  misc_feature 1..146: note = Synthetic
  source 1..146: mol_type = other DNA
tcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaata

SEQ ID NO: 36 - DNA, 468 bp, synthetic construct
  misc_feature 1..468: note = Synthetic
  source 1..468: mol_type = other DNA
gggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatc

SEQ ID NO: 37 - DNA, 10101 bp, synthetic construct
  misc_feature 1..10101: note = Synthetic
  source 1..10101: mol_type = other DNA
caggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcgcgcaattaaccctcactaaagggaacaaaagctggagctgcaagcttggccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccggggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacctgaaagcgaaagggaaaccagagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcctcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcggttaacttttaaaagaaaaggggggattggggggtacagtgcaggggaaagaatagtagacataatagcaacagacatacaaactaaagaattacaaaaacaaattacaaaaattcaaaattttatcgatcacgagactagcctcgacggtatcgataagcttgatgatatcccatagagcccaccgcatccccagcatgcctgctattgtcttcccaatcctcccccttgctgtcctgccccaccccaccccccagaatagaatgacacctactcagacaatgcgatgcaatttcctcattttattaggaaagggcagtgggagtggcaccttccagggtcaaggaaggcacgggggaggggcaaacaacagatggctggcaactagaaggcacagcgtacggtcatgtagtgtttcctactttatggagtgatgtagtgtttcctactttatggaaccggttgtagtgtttcctactttatggaatcgtgtagtgtttcctactttatggaatcggtcgaggtttaaactcatgccacatcgtcctccaggctgaccatctgtcttgaggggtaggtatagccatcctggcttcggcaaatgtggagctcgtcctctgtggtcttctggtagactgggttgtcaaagtttatgctgttgatgttcttcagccgccagttcctccacagcaggacggccccaaggacaaggagggcaaccagtgcaatagggaagaagatggacaggaacctcataccatgtggctgctcctcattccctctgccagccatgtcaccctggacttggtgggacactgtcactgacgccactgtgctgagccctggggtgtccacaggctgcctaggagtactgggagctgagagatcctcactgtgcttcggtggcctggtagctgatgcggtgaccacaggccggacggcggatgtcccctgggtggtcagtacagtgtcgacttctgtgaggcagctccgcatgtccttggccagcagcatgccatcagggcaggcgcaggtgaatttgggcgagtggggaccgatctgtggggcgggcaggcacaggtactggcaaccaccattggggaggagggctgttgtctcacaccagttcacccctctaggctgtgtgaccttgtggaacaggacaatgtcctccggggacaagaggttttcagccaccaaattcacatctgaacccgtgagtcgattggcactgaaaatggcttcgtttatgacatctgtccaatacactttgtcctcatagatggccaaggagaaggggtgggccagccggttctcatcctccaaaatggttttccgattgcccccattgacatcgatgctggagatagagtggagtttggaatcaacccaatagagacggccactggaaagatctagtgtgatgccatttggccactggatgttttcggtcaccagtgagtggatgtccacaccattcaaaccccctttcttgatcttggcgggtgttccccaatctgtccagtacatgaagccatgcacagggtccactacgatggctctgggtctggaccctgcctcttggaacagtgtcctcctctttacgcccttggtgtcagccacagatacgctgcctgggactgaatctgtccagtagatgttgcggtggatccagtctaccgccagcccgtcaggggcatgcaggtcctcactgatgatggtgtcgtaggacaagttaggggcctggtccatcagggcgctgtagatctttttttgggacaggtcggaccagtagattctattgttggtcacctccgtgtcgagagccaccacattcttcaggttggggagcagactggtgtactcgctgcggtccagggtcatcttccggacctcgtggcggttggtgaagagcagatagcctatggagcccacagccttgcagaccctggtgtgtgggtccatgtggaagccggcctggcactcacacttgtagctgccttccaggttcacacagagctggctgcaggtgtctggctcctgacactcgtcaatatcttcacacctgtggaggtccaccaaccggaagccgctgggacacaggcactcagagccaatcttgaggtccttgcagatgtgggaacagccaccattgttgtccaaacactcgttggtcttgcactccttgatgggctcatccgaccagtcctggcagtcgcgggcggagtcgcacaccttgtccaagctgatgcactccccactgtgacacttgaacttgttggggccatcacactgtgtcacattgacgcagccgagctcgtcgctcatgtccttgcagtcatgttcacggtcacactggcggctaccgtgaatgcaggagccatctgcacactggaattcatcaggtcggcaggtggccaccgcgcagtgctcctcatctgacttgtccttgcagtctgcctcgccgtcacagacccagctgcgatggatacactcactgctaccacagtggaactccagggaggagcaggggctgctaacgcctttggaggccgtgtctcggccctggcagttctgtggccactcatcggagccgtcaacacagtcgacatccccgtcgcaggcccaaagactggggatgcatatggatgagttgcagcggaagtgggcggggccacaagtggtggcctggcagtgggcctcatcagagccatctaggcaatctcggtctccatcacacacaaactgcggggagatgcacttgccatcctggcatcggaagtcatcctgggagcacgtcttggggggacagccttgttcgtctgagtcattttcacagtctacctgtccatcacatctccaggagtcaggaatgcatcggctgacacggcctccacagctgaattgattggactgacaggtgacagacatgcatgtctctggggactcatcggagccatccgggcactcggggctgccatcgcacacccacttgctagcgatgcattttccgtctctacactggaactcgttcctgctgcatgagtcttctgctgcaactccggcagcagccaggagcagggcgatgacccagcgacgcatcagatccgcggtgctcatggtggcacgcgttccggtggatccactagccaggagcttgtggatctgtgtgacggcttctcctggtgaaggggcttttataccccctccttccaacccaggctgctgatccctgccaagctgactccaaacctgctgattctgattattgacttagtcaacaaaaggagaataagtaacctacacaaatatgaaccttgcctagggagattagagtatcggaacactcgctctacgaaatgtgcagacagacggggatcctctagagctacctgctgatcgcccggcccctgttcaaacatgtcctaatactctgtctctgcaagggtcatcagtagttttccatcttactcaacatcctcccagtgccctcgacctgcagcccaagctcctaagcttatcgataccgtccggggcggggcgaacgcgcgctgggcaaaggtcacctgcccctcgcgattgcggcaatcgcggcctcgaatcaatattcgcgaggttaataattaccagcgcgcgctgggcaaacgtcacctgcccctcgcggattatttatttggcccgcgctggtaattattaactcgcgtgctcgacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctggaattcgagctcggtacctttaagaccaatgacttacaaggcagctgtagatcttagccactttttaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagtatttataacttgcaaagaaatgaatatcagagagtgagaggaacttgtttattgcagcttataatggttacaaataaagcaatagcatcacaaatttcacaaataaagcatttttttcactgcattctagttgtggtttgtccaaactcatcaatgtatcttatcatgtctggctctagctatcccgcccctaactccgcccagttccgcccattctccgccccatggctgactaattttttttatttatgcagaggccgaggccgcctcggcctctgagctattccagaagtagtgaggaggcttttttggaggcctaggcttttgcgtcgagacgtacccaattcgccctatagtgagtcgtattacgcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcgacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttcc

SEQ ID NO: 38 - protein, 862 aa, synthetic construct
  REGION 1..862: note = Synthetic
MSTADLMRRWVIALLLAAAGVAAEDSCSRNEFQCRDGKCIASKWVCDGSPECPDGSDESPETCMSVTCQSNQFSCGGRVSRCIPDSWRCDGQVDCENDSDEQGCPPKTCSQDDFRCQDGKCISPQFVCDGDRDCLDGSDEAHCQATTCGPAHFRCNSSICIPSLWACDGDVDCVDGSDEWPQNCQGRDTASKGVSSPCSSLEFHCGSSECIHRSWVCDGEADCKDKSDEEHCAVATCRPDEFQCADGSCIHGSRQCDREHDCKDMSDELGCVNVTQCDGPNKFKCHSGECISLDKVCDSARDCQDWSDEPIKECKTNECLDNNGGCSHICKDLKIGSECLCPSGFRLVDLHRCEDIDECQEPDTCSQLCVNLEGSYKCECQAGFHMDPHTRVCKAVGSIGYLLFTNRHEVRKMTLDRSEYTSLLPNLKNVVALDTEVTNNRIYWSDLSQKKIYSALMDQAPNLSYDTIISEDLHAPDGLAVDWIHRNIYWTDSVPGSVSVADTKGVKRRTLFQEAGSRPRAIVVDPVHGFMYWTDWGTPAKIKKGGLNGVDIHSLVTENIQWPNGITLDLSSGRLYWVDSKLHSISSIDVNGGNRKTILEDENRLAHPFSLAIYEDKVYWTDVINEAIFSANRLTGSDVNLVAENLLSPEDIVLFHKVTQPRGVNWCETTALLPNGGCQYLCLPAPQIGPHSPKFTCACPDGMLLAKDMRSCLTEVDTVLTTQGTSAVRPVVTASATRPPKHSEDLSAPSTPRQPVDTPGLSTVASVTVSHQVQGDMAGRGNEEQPHGMRFLSIFFPIALVALLVLGAVLLWRNWRLKNINSINFDNPVYQKTTEDELHICRSQDGYTYPSRQMVSLEDDVA

SEQ ID NO: 39 - DNA, 2589 bp, synthetic construct
  misc_feature 1..2589: note = Synthetic
  source 1..2589: mol_type = other DNA
atgagcaccgcggatctgatgcgtcgctgggtcatcgccctgctcctggctgctgccggagttgcagcagaagactcatgcagcaggaacgagttccagtgtagagacggaaaatgcatcgctagcaagtgggtgtgcgatggcagccccgagtgcccggatggctccgatgagtccccagagacatgcatgtctgtcacctgtcagtccaatcaattcagctgtggaggccgtgtcagccgatgcattcctgactcctggagatgtgatggacaggtagactgtgaaaatgactcagacgaacaaggctgtccccccaagacgtgctcccaggatgacttccgatgccaggatggcaagtgcatctccccgcagtttgtgtgtgatggagaccgagattgcctagatggctctgatgaggcccactgccaggccaccacttgtggccccgcccacttccgctgcaactcatccatatgcatccccagtctttgggcctgcgacggggatgtcgactgtgttgacggctccgatgagtggccacagaactgccagggccgagacacggcctccaaaggcgttagcagcccctgctcctccctggagttccactgtggtagcagtgagtgtatccatcgcagctgggtctgtgacggcgaggcagactgcaaggacaagtcagatgaggagcactgcgcggtggccacctgccgacctgatgaattccagtgtgcagatggctcctgcattcacggtagccgccagtgtgaccgtgaacatgactgcaaggacatgagcgacgagctcggctgcgtcaatgtgacacagtgtgatggccccaacaagttcaagtgtcacagtggggagtgcatcagcttggacaaggtgtgcgactccgcccgcgactgccaggactggtcggatgagcccatcaaggagtgcaagaccaacgagtgtttggacaacaatggtggctgttcccacatctgcaaggacctcaagattggctctgagtgcctgtgtcccagcggcttccggttggtggacctccacaggtgtgaagatattgacgagtgtcaggagccagacacctgcagccagctctgtgtgaacctggaaggcagctacaagtgtgagtgccaggccggcttccacatggacccacacaccagggtctgcaaggctgtgggctccataggctatctgctcttcaccaaccgccacgaggtccggaagatgaccctggaccgcagcgagtacaccagtctgctccccaacctgaagaatgtggtggctctcgacacggaggtgaccaacaatagaatctactggtccgacctgtcccaaaaaaagatctacagcgccctgatggaccaggcccctaacttgtcctacgacaccatcatcagtgaggacctgcatgcccctgacgggctggcggtagactggatccaccgcaacatctactggacagattcagtcccaggcagcgtatctgtggctgacaccaagggcgtaaagaggaggacactgttccaagaggcagggtccagacccagagccatcgtagtggaccctgtgcatggcttcatgtactggacagattggggaacacccgccaagatcaagaaagggggtttgaatggtgtggacatccactcactggtgaccgaaaacatccagtggccaaatggcatcacactagatctttccagtggccgtctctattgggttgattccaaactccactctatctccagcatcgatgtcaatgggggcaatcggaaaaccattttggaggatgagaaccggctggcccaccccttctccttggccatctatgaggacaaagtgtattggacagatgtcataaacgaagccattttcagtgccaatcgactcacgggttcagatgtgaatttggtggctgaaaacctcttgtccccggaggacattgtcctgttccacaaggtcacacagcctagaggggtgaactggtgtgagacaacagccctcctccccaatggtggttgccagtacctgtgcctgcccgccccacagatcggtccccactcgcccaaattcacctgcgcctgccctgatggcatgctgctggccaaggacatgcggagctgcctcacagaagtcgacactgtactgaccacccaggggacatccgccgtccggcctgtggtcaccgcatcagctaccaggccaccgaagcacagtgaggatctctcagctcccagtactcctaggcagcctgtggacaccccagggctcagcacagtggcgtcagtgacagtgtcccaccaagtccagggtgacatggctggcagagggaatgaggagcagccacatggtatgaggttcctgtccatcttcttccctattgcactggttgccctccttgtccttggggccgtcctgctgtggaggaactggcggctgaagaacatcaacagcataaactttgacaacccagtctaccagaagaccacagaggacgagctccacatttgccgaagccaggatggctatacctacccctcaagacagatggtcagcctggaggacgatgtggcatga

SEQ ID NO: 40 - protein, 692 aa, synthetic construct
MGTVSSRRSWWPLPLLLLLLLLLGPAGARAQEDEDGDYEELVLALRSEEDGLAEAPEHGTTATFHRCAKDPWRLPGTYVVVLKEETHLSQSERTARRLQAQAARRGYLTKILHVFHGLLPGFLVKMSGDLLELALKLPHVDYIEEDSSVFAQSIPWNLERITPPRYRADEYQPPDGGSLVEVYLLDTSIQSDHREIEGRVMVTDFENVPEEDGTRFHRQASKCDSHGTHLAGVVSGRDAGVAKGASMRSLRVLNCQGKGTVSGTLIGLEFIRKSQLVQPVGPLVVLLPLAGGYSRVLNAACQRLARAGVVLVTAAGNFRDDACLYSPASAPEVITVGATNAQDQPVTLGTLGTNFGRCVDLFAPGEDIIGASSDCSTCFVSQSGTSQAAAHVAGIAAMMLSAEPELTLAELRQRLIHFSAKDVINEAWFPEDQRVLTPNLVAALPPSTHGAGWQLFCRTVWSAHSGPTRMATAVARCAPDEELLSCSSFSRSGKRRGERMEAQGGKLVCRAHNAFGGEGVYAIARCCLLPQANCSVHTAPPAEASMGTRVHCHQQGHVLTGCSSHWEVEDLGTHKPPVLRPRGQPNQCVGHREASIHASCCHAPGLECKVKEHGIPAPQEQVTVACEEGWTLTGCSALPGTSHVLGAYAVDNTCVVRSRDVSTTGSTSEGAVTAVAICCRSRHLAQASQELQ

SEQ ID NO: 41 - protein, 692 aa, synthetic construct
MGTVSSRRSWWPLPLLLLLLLLLGPAGARAQEDEDGDYEELVLALRSEEDGLAEAPEHGTTATFHRCAKDPWRLPGTYVVVLKEETHLSQSERTARRLQAQAARRGYLTKILHVFHGLLPGFLVKMRGDLLELALKLPHVDYIEEDSSVFAQSIPWNLERITPPRYRADEYQPPDGGSLVEVYLLDTSIQSDHREIEGRVMVTDFENVPEEDGTRFHRQASKCDSHGTHLAGVVSGRDAGVAKGASMRSLRVLNCQGKGTVSGTLIGLEFIRKSQLVQPVGPLVVLLPLAGGYSRVLNAACQRLARAGVVLVTAAGNFRDDACLYSPASAPEVITVGATNAQDQPVTLGTLGTNFGRCVDLFAPGEDIIGASSDCSTCFVSQSGTSQAAAHVAGIAAMMLSAEPELTLAELRQRLIHFSAKDVINEAWFPEDQRVLTPNLVAALPPSTHGAGWQLFCRTVWSAHSGPTRMATAVARCAPDEELLSCSSFSRSGKRRGERMEAQGGKLVCRAHNAFGGEGVYAIARCCLLPQANCSVHTAPPAEASMGTRVHCHQQGHVLTGCSSHWEVEDLGTHKPPVLRPRGQPNQCVGHREASIHASCCHAPGLECKVKEHGIPAPQEQVTVACEEGWTLTGCSALPGTSHVLGAYAVDNTCVVRSRDVSTTGSTSEGAVTAVAICCRSRHLAQASQELQ

SEQ ID NO: 42 - DNA, 2079 bp, synthetic construct
  source 1..2079: mol_type = other DNA
atgggcaccgtgtccagcagacggtcttggtggcctctgcctctgctgttgctgctgcttcttctgcttggacctgctggcgctagagcccaagaggatgaggatggcgactacgaggaactggtgctggccctgagaagcgaggaagatggactggccgaagctcctgagcacggcaccacagccaccttccacagatgcgccaaagatccttggagactgcccggcacatacgtggtggtgctgaaagaggaaacccacctgagccagagcgagagaacagccagaaggctgcaggctcaggccgccagaagaggctacctgaccaagatcctgcacgtgttccacggcctgctgcctggctttctggtcaagatgtctggcgatctgctggaactggctctgaagctgcctcacgtggactacatcgaagaggacagcagcgtgttcgcccagagcatcccctggaacctggaaagaatcacccctcctagataccgggccgacgagtaccaacctcctgatggcggatctctggtggaagtgtacctgctggataccagcatccagagcgaccaccgggaaatcgagggcagagtgatggtcaccgacttcgagaacgtgcccgaagaagatggcacccggtttcacagacaggccagcaagtgtgatagccacggaacacatctggccggcgtggtgtctggaagagatgctggtgttgccaagggcgccagcatgagatctctgagagtgctgaactgccaaggcaagggcacagtgtctggcacactgatcggcctcgagttcatccggaagtcccagctggttcagcctgtgggacctctggttgttctgctgccactggctggcggctatagcagggttctgaatgccgcctgtcagagactggctagagctggcgttgtgctggttacagccgccggaaacttcagagatgacgcctgcctgtacagccctgccagtgctcctgaagtgatcacagtgggcgccacaaacgcccaggatcagcctgttacactgggcaccctgggcacaaacttcggcagatgcgtggacctgtttgcccctggcgaggatattatcggcgccagctccgattgcagcacctgttttgtgtctcagagcggcacctctcaggctgccgctcatgttgctggaatcgccgccatgatgctgtctgccgagcctgaactgactctggccgagctgagacagcggctgatccactttagcgccaaggacgtgatcaacgaggcctggtttcccgaggatcagagggtgctgacccctaatctggtggctgctctgccaccttctacacacggtgctggctggcagctgttctgcaggacagtttggagcgcccacagcggccctacaagaatggctacagccgtggctagatgcgcccctgatgaggaactgctgagctgctccagcttcagcagaagcggcaagagaagaggcgagcggatggaagcccaaggcggaaaacttgtgtgcagagcccacaatgcctttggcggagaaggcgtgtacgccattgccagatgttgtctgttgccccaggccaactgcagcgtgcacacagctcctccagccgaagcctctatgggcaccagagtgcactgtcaccagcagggacatgtgctgacaggctgtagcagccactgggaagttgaggacctgggaacccacaagcctccagtgctcagacctagaggccagcctaatcagtgcgtgggacacagagaggcctccatccacgcctcttgttgtcatgcccctggactggaatgcaaagtgaaagagcacggaatccccgctcctcaagagcaagtgaccgtggcctgtgaagaaggctggacactgaccggatgttctgccctgcctggcacatctcatgtgctgggagcctacgccgtggacaatacctgtgttgtgcggagcagagatgtgtccaccaccggctctacatctgagggcgctgtgacagctgtggccatctgctgcagaagcagacacctggcacaggcctctcaagagctgcagtga

SEQ ID NO: 43 - DNA, 2079 bp, synthetic construct
  source 1..2079: mol_type = other DNA
atgggcaccgtgagctcccggagaagctggtggcctctgccactgttattactgctgctgctgctgctgggaccagcaggagcaagggcccaggaggacgaggatggcgactacgaggagctggtgctggccctgcgctccgaggaggacggcctggccgaggcccctgagcacggcaccacagccaccttccacaggtgcgcaaaggacccctggaggctgccaggcacatacgtggtggtgctgaaggaggagacacacctgtcccagtctgagaggaccgcaaggcgcctgcaggcacaggcagcaaggagaggctatctgaccaagatcctgcacgtgttccacggcctgctgccaggctttctggtgaagatgaggggcgacctgctggagctggccctgaagctgccacacgtggattacatcgaggaggactctagcgtgtttgcccagtctatcccctggaacctggagagaatcaccccccctcggtacagagccgatgagtatcagccaccagacggaggctccctggtggaggtgtatctgctggatacaagcatccagtccgaccaccgggagatcgagggcagagtgatggtgacagacttcgagaacgtgcctgaggaggatggcaccaggtttcaccgccaggcctctaagtgcgacagccacggcacccacctggcaggagtggtgagcggccgggatgcaggagtggcaaagggagcatctatgcggagcctgagagtgctgaattgtcagggcaagggcacagtgtccggcaccctgatcggcctggagttcatccggaagtctcagctggtgcagccagtgggaccactggtggtgctgctgccactggcaggaggatacagcagagtgctgaacgcagcatgccagaggctggcaagggcaggcgtggtgctggtgacagccgccggcaacttccgggacgatgcctgtctgtattcccccgcctctgcccctgaggtgatcacagtgggagcaaccaacgcacaggaccagcctgtgaccctgggcacactgggcaccaatttcggccgctgcgtggatctgtttgcaccaggagaggacatcatcggagcatcctctgattgcagcacatgtttcgtgagccagtccggcacctcccaggctgccgcccacgtggcaggaatcgcagcaatgatgctgagcgccgagccagagctgaccctggcagagctgaggcagcgcctgatccacttctccgccaaggacgtgatcaacgaggcctggtttcctgaggatcagagggtgctgacaccaaatctggtggccgccctgcctccaagcacccacggagccggctggcagctgttttgtagaacagtgtggagcgcccactccggaccaacaaggatggcaaccgcagtggcaagatgcgcacctgacgaggagctgctgtcctgtagctccttctctaggagcggaaagaggaggggagagaggatggaggcacagggaggaaagctggtgtgcagggcacacaacgcctttggcggagagggcgtgtacgcaatcgcaagatgctgtctgctgcctcaggccaattgttctgtgcacacagcaccacctgcagaggcaagcatgggaaccagggtgcactgccaccagcagggacacgtgctgaccggctgttctagccactgggaggtggaggatctgggaacacacaagccacccgtgctgcggccaagaggacagccaaaccagtgcgtgggccacagagaggcctccatccacgcctcttgctgtcacgccccaggcctggagtgtaaggtgaaggagcacggcatccccgcacctcaggagcaggtgaccgtggcatgcgaggagggatggaccctgacaggatgttctgccctgccaggcaccagccacgtgctgggagcatatgcagtggacaatacatgcgtggtgaggagccgcgacgtgagcaccacaggctccacatctgagggagcagtgaccgcagtggcaatctgctgtcggtccagacacctggcccaggcctctcaggagctgcagtga

SEQ ID NO: 44 - DNA, 17 bp, synthetic construct
  misc_feature 1..17: note = Synthetic
  source 1..17: mol_type = other DNA
actgacgctctcgcacc

SEQ ID NO: 45 - DNA, 17 bp, synthetic construct
  misc_feature 1..17: note = Synthetic
  source 1..17: mol_type = other DNA
tctcgacgcaggactcg

SEQ ID NO: 46 - DNA, 20 bp, synthetic construct
  misc_feature 1..20: note = Synthetic
  source 1..20: mol_type = other DNA
  misc_feature 1: note = FAM label linked at 5' end
atctctctccttctagcctc

SEQ ID NO: 47 - DNA, 20 bp, synthetic construct
  misc_feature 1..20: note = Synthetic
  source 1..20: mol_type = other DNA
accgattccagatgattggc

SEQ ID NO: 48 - DNA, 22 bp, synthetic construct
  misc_feature 1..22: note = Synthetic
  source 1..22: mol_type = other DNA
tccatattaatgcagtgcttgc

SEQ ID NO: 49 - DNA, 24 bp, synthetic construct
  misc_feature 1..24: note = Synthetic
  source 1..24: mol_type = other DNA
  misc_feature 1: note = HEX label linked at 5' end
  misc_feature 24: note = BHQ1 label linked at 3' end
agaggcctgtcctgcagctcatgg